(19) United States
(12) Patent Application Publication
(10) Pub. No.: US 2026/0066047 A1
(43) Pub. Date: Mar. 5, 2026

(54) MODELS AND METHODS FOR PREDICTING CELL-SURFACE PROTEIN EXPRESSION

(71) Applicant: Wisconsin Alumni Research Foundation, Madison, WI (US)

(72) Inventor: Shuang Zhao, Verona, WI (US)

(73) Assignee: Wisconsin Alumni Research Foundation, Madison, WI (US)

(21) Appl. No.: 19/317,440

(22) Filed: Sep. 3, 2025

Related U.S. Application Data
(60) Provisional application No. 63/690,389, filed on Sep. 4, 2024.

(51) Int. Cl. G16B 25/10; G16B 45/00; G16B 50/30; G16H 10/40; G16H 10/60

(52) U.S. Cl. CPC G16B 25/10; G16B 45/00; G16B 50/30; G16H 10/40; G16H 10/60

(57) ABSTRACT

Models and methods for predicting cell-surface protein expression, such as expression of cell-surface targets on cancer cells.

### CROSS-REFERENCE TO RELATED APPLICATIONS

Priority is hereby claimed to U.S. Provisional Application 63/690,389, filed Sep. 4, 2024, which is incorporated herein by reference in its entirety.

### FIELD OF THE INVENTION

The invention is directed to models and methods for predicting cell-surface protein expression, such as expression of cell-surface targets on cancer cells.

### BACKGROUND

Cell surface targeted therapies have emerged as a highly effective new class of therapies for both hematologic and solid tumor malignancies. Unlike conventional cytotoxic chemotherapy, therapies targeting cell surface proteins offer a strategy to increase cancer cell specificity while minimizing toxicities. Multiple types of cell surface protein-targeted therapies have been developed with different mechanisms of action. Antibody-Drug Conjugates (ADCs), which selectively deliver cytotoxic payloads to tumor cells via antibodies against tumor cell surface targets, represent a highly successful class with multiple FDA approvals. Several other classes of cell surface targeted therapies have received FDA approval, including Radiopharmaceutical Therapies (RPTs), which deliver a therapeutic radionuclide, as well as a number of immune modulatory cell surface targeted therapies including Chimeric Antigen Receptor T-cells (CAR-Ts), CAR natural killer cells (CAR-NKs), CAR dendritic cells (CAR-DCs), CAR macrophages (CAR-Ms), and Bi-specific T-cell Engagers (BiTEs).

In solid tumors, there are nine currently FDA-approved cell surface targeted therapies.

ADCs are the most common class, with T-DM1 (targeting HER2) approved in HER2+ breast cancer, fam-trastuzumab deruxtecan (targeting HER2) approved for HER2+ solid cancers and HER2 mutant non-small cell lung cancer (NSCLC), enfortumab vedotin (targeting NECTIN4) approved in bladder cancer, sacituzumab govitecan, (targeting TROP2) approved for HER2-negative breast cancer and bladder cancer, mirvetuximab soravtansine (targeting FOLR1) approved for FOLR1 positive platinum-resistant epithelial ovarian, fallopian tube, and peritoneal cancers, and tisotumab vedotin (targeting tissue factor) approved for cervical cancer. RPTs are the next most common, with ¹⁷⁷Lu-PSMA-617 (targeting PSMA) approved for prostate cancer and ¹⁷⁷Lu-DOTATATE (targeting SSTR2 approved for somatostatin receptor-positive gastroenteropancreatic neuroendocrine tumors. Finally, tebentafusp-tebn is a BiTE (targeting gp100) approved for uveal melanoma and is in trials for cutaneous melanoma. Tarlatamab, a DLL3 BiTE was also approved recently for small-cell lung cancer (SCLC). Dozens of other cell surface targeted therapies for these and other proteins are in clinical trials. This class of agents represents an important emerging addition to our anti-neoplastic armamentarium. As the potential options multiply, a method to prioritize between a multitude of potential targets is needed.

### SUMMARY OF THE INVENTION

One aspect of the invention is directed to methods of generating a prediction model of cell-surface protein expression in cancer.

In some versions, the methods comprise a step of determining a gene expression profile for each training cell sample in a set of training cell samples. In some versions, each gene expression profile comprises a set of gene-expression values for a set of gene-expression-profile genes. In some versions, the set of training cell samples comprises a set of training cancer cell samples.

In some versions, the methods further comprise a step of determining a set of training genes. In some versions, the set of training genes comprises a common set of gene-expression-profile genes represented in each of the gene expression profiles. In some versions, the set of training genes comprises a set of cell-surface protein genes.

In some versions, the methods further comprise a step of ranking the gene-expression values for all of the training genes in each expression profile to thereby obtain a training ranking for each gene expression profile. In some versions, each training ranking comprises a rank for the gene-expression value for each training gene within the gene expression profile.

In some versions, the methods further comprise a step of identifying, for each training gene, the training cell samples having a same rank.

In some versions, the ranking comprises ordinally ranking the gene-expression values for all of the training genes in each gene expression profile to thereby obtain an ordinal training ranking for each gene expression profile, wherein each ordinal training ranking comprises an ordinal rank for the gene-expression value for each training gene within the gene expression profile.

In some versions, the ranking further comprises: determining a number of genes in the set of training genes; and transforming each ordinal ranking into a quantile ranking by dividing the ordinal rank for the gene-expression value for each gene within the gene expression profile by the number of genes in the set of training genes.

In some versions, the gene expression profiles are determined through RNA-Seq, gene expression microarray, or a combination thereof.

In some versions, the set of cell-surface protein genes comprises of at least 1, at least 5, at least 10, at least 15, at least 20, at least 25, at least 30, at least 35, at least 40, at least 45, at least 50, at least 55, at least 60, at least 65, at least 70, or each of FOLH1, MUC1, MAGEA3, NCAM1, CD276, EPCAM, GPNMB, ERBB2, CEACAM5, CD70, CA6, SLC44A4, TNC, MSLN, TNFRSF8, EGFR, GUCY2C, FOLR1, TACSTD2, TACSTD2, ENPP3, MET, DLL3, SLC39A6, F3, EFNA4, NECTIN4, KIT, GPA33, FGFR3, PROM1, LRRC15, ROR1, PSCA, HAVCR1, TFRC, GPC3, CDH6, ERBB3, AXL, ALCAM, PTK7, SLC34A2, SSTR2, LY75, ROR2, MUC16, CD46, ISG20, IGF1R, GRPR, ROBO1, TNFRSF10B, TNFRSF9, STEAP1, CLDN18, ITGB6, ICAM1, TPBG, TYRP1, CD22, CD274, HLA-G, CD83, CLDN6, VTCN1, FAP, FLT1, CXCR4, EPHA2, CA9, EPHA5, PMEL, TM4SF1, ADAM9, CD38, FN1, CEACAM6, and NCR2.

In some versions, the set of training genes further comprises housekeeping genes.

In some versions, the set of training genes comprises at least 500 genes, at least 1,000 genes, at least 2,500 genes, at least 5,000 genes, at least 7,500 genes, at least 10,000 genes, at least 12,500 genes, at least 15,000 genes, at least 17,500 genes, or at least 19,000 genes.

In some versions, the set of training cancer cell samples comprise primary tumor cell samples, metastatic tumor cell samples, or both primary tumor cell samples and metastatic tumor cell samples.

In some versions, the training cancer cell samples comprise a type of at least 1, at least 5, at least 10, at least 15, at least 20, at least 25, at least 30, at least 35, at least 40, at least 45, or each of adrenocortical carcinoma, anal squamous cell carcinoma, bladder urothelial carcinoma, breast invasive carcinoma (basal subtype), breast invasive carcinoma (HER2 subtype), breast invasive carcinoma (luminal A subtype), breast invasive carcinoma (luminal B subtype), cholangiocarcinoma, colorectal adenocarcinoma, cervical squamous cell carcinoma, cancer of unknown primary, diffuse large B-cell lymphoma, endocervical adenocarcinoma, esophagus squamous cell carcinoma, fibrolamellar carcinoma, glioblastoma multiforme, gastrointestinal stromal tumor, adenoid cystic carcinoma, head and neck squamous cell carcinoma, kidney chromophobe, kidney renal clear cell carcinoma, kidney renal papillary cell carcinoma, acute myeloid leukemia, brain lower grade glioma, liver hepatocellular carcinoma, lung adenocarcinoma, lung squamous cell carcinoma, mesothelioma, miscellaneous, prostate neuroendocrine carcinoma, clear cell ovarian cancer, ovarian serous cystadenocarcinoma, pancreatic adenocarcinoma, pheochromocytoma and paraganglioma, prostate adenocarcinoma, sarcoma, small bowel adenocarcinoma, small cell lung cancer, other small cell or neuroendocrine tumor, skin cutaneous melanoma, testicular germ cell tumor, thyroid carcinoma, thymoma, uterine corpus endometrial carcinoma, uterine carcinosarcoma, esophagus/stomach adenocarcinoma, and uveal melanoma.

In some versions, the set of training cell samples further comprises training non-cancer cell samples.

In some versions, the training cell samples include cell lines.

In some versions, the training cell samples comprise single cell samples.

In some versions, the single cell samples comprise single cancer cells and single non-cancer cells.

In some versions, the methods further comprise: generating a matrix data structure comprising a plurality of rows as the training cell samples and columns as the cancer cell samples; and storing the matrix data structure in computer memory.

In some versions, the matrix data structure is stored in contiguous memory in RAM.

In some versions, the methods further comprise communicating the matrix data structure from the computer memory to a networked database.

In some versions, the methods further comprise, prior to determining the gene expression profile for each training cell sample, pre-processing the set of training cell samples, wherein pre-processing comprises normalizing for gene length and sequencing depth.

In some versions, the normalizing is based on at least one of Transcripts Per Kilobase Million (TPM), Fragments Per Kilobase Million (FPKM), or Reads Per Kilobase Million (RPKM).

In some versions, the methods further comprise, prior to ranking the gene-expression values for all of the training genes in each expression profile, normalizing for gene length and sequencing depth.

In some versions, the set of training cell samples is missing at least one piece of data, and wherein the normalizing accounts for the missing at least one piece of data.

Another aspect of the invention is directed to methods using a prediction model of the invention to determine a predicted cell-surface target on a patient cancer cell sample. In some versions, the methods comprise ranking gene-expression values of all of the training genes in the patient cancer cell sample to obtain a patient ranking; selecting one or more training genes having a threshold rank in the patient ranking to thereby obtain one or more patient genes; comparing the rank(s) of the one or more patient genes in the patient cancer cell sample with the rank(s) of the one or more patient genes in the training cell samples to determine a number, type, and/or proportion of training cancer cell samples having the same rank(s) for the one or more patient genes; and optionally, empirically testing cell-surface expression of the one or more patient genes in a test cancer cell sample from the patient.

In some versions, the comparing further comprises determining a number, type, and/or proportion of non-cancer cell samples having the same rank(s) for the one or more patient genes.

Some versions further comprise, prior to ranking the gene-expression values for all of the training genes in each expression profile, normalizing for gene length and sequencing depth.

In some versions, the set of training cell samples is missing at least one piece of data, and wherein the normalizing accounts for the missing at least one piece of data.

Some versions further comprise: presenting a graphical user interface to a user of the compared rank(s) of the one or more patient genes in the patient cancer cell sample with the rank(s) of the one or more patient genes in the training cell samples as a plurality of top cell surface targets organized by percentile.

Some versions further comprise generating a pseudo-dynamic graphical user interface that simulates a dynamic user experience by: pre-generating an image of all possible integer percentiles for a given training cancer cell as a plurality of images; and populating the graphical user interface with a given image based on user input of an inputted training gene and an inputted percentile.

In some versions, the graphical user interface is populated with a given image without using an index data structure.

In some versions, each of the plurality of images comprise a filename including training gene and percentile.

Another aspect of the invention is directed to methods of using a prediction model of the invention to identify a cancer type expressing a cell-surface protein. In some versions, the methods comprise: determining the ranks of one of the cell-surface protein genes across the training cancer cell samples of different types; selecting one of the different types of training cancer cell samples having a threshold rank for the one of the cell-surface protein genes; and optionally, empirically testing cell-surface expression of the one of the cell-surface protein genes in a test cancer cell sample.

Another aspect of the invention is directed to methods of using a prediction model of the invention to identify a model cell line for testing a cell-surface targeted therapy in a particular cancer cell type. In some versions, the methods comprise: identifying types of the training cancer cell samples having a threshold rank of one of the cell-surface protein genes; identifying a first set of cell lines from the training cell samples having a rank of the one of the cell-surface protein genes comparable to the identified types of the training cancer cell samples; identifying a second set of cell lines from the training cell samples that have a threshold level of protein expression of one of the cell-surface protein genes; identifying the cell lines common to the first set of cell lines and the second set of cell lines to thereby obtain one or more model cell lines; and optionally, testing a therapy targeting the cell-surface protein gene on the one or more model cell lines.

Another aspect of the invention is directed to methods of using a prediction model of the invention to predict response and/or toxicity in the treatment of a particular type of cancer. In some versions, the methods comprise: identifying a proportion of single cells of a training cancer cell sample from the particular type of cancer having higher than a threshold ranking of one of the cell-surface protein genes; and/or identifying a proportion of single cells of a training non-cancer cell sample having lower than a threshold ranking of the one of the cell-surface protein genes; and optionally, treating the particular type of cancer with a therapeutic targeting the one of the cell-surface protein genes if the proportion of single cells of the training cancer cell sample from the particular type of cancer is higher than a threshold and/or if the proportion of single cells of the training non-cancer cell sample is lower than a threshold.

Another aspect of the invention is directed to methods of using a prediction model of the invention to identify two or more cell-surface targets on a particular type of cancer. In some versions, the methods comprise: generating a logistic regression model for every pair of the cell-surface protein genes comparing each type of training cancer cell sample and each type of training non-cancer cell sample; identifying pairs in which both cell-surface protein genes in the pair individually contribute to predicting a training cancer cell sample versus a training non-cancer cell sample to obtain predictive pairs; and identifying predictive pairs in which the combination of both cell-surface protein genes in the pair discriminate between a training cancer cell sample versus a training non-cancer cell sample.

The objects and advantages of the invention will appear more fully from the following detailed description of the preferred embodiment of the invention made in conjunction with the accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1. A block diagram of a system for generating a prediction model of cell-surface protein expression in cancer, according to an embodiment.

FIG. 2. A functional block diagram of a system for generating a prediction model of cell-surface protein expression in cancer, according to an embodiment.

FIGS. 3A-3C. Screenshots of graphical user interfaces presented by a system for generating a prediction model of cell-surface protein expression in cancer, according to embodiments.

FIGS. 4A-4E. Cell surface target tumor expression. FIG. 4A Schematic of all data used in this study. FIGS. 4B-4E Hierarchical clustering of median bulk RNA expression of 78 cell surface protein targets (rows) across 43 solid cancer types (columns; primary and metastatic). RNA expression levels are percentile rank normalized across all genes, ranging from 0 to 1. Red star=FDA approved indication. FIGS. 4B-4E show four portions of a single hierarchical clustering, with FIGS. 4B-4E continuing in order from top to bottom.

FIGS. 5A-5C. Primary vs. metastatic tumor expression. A Rank-Biserial correlation was calculated between the primary vs. metastatic samples in 23 tumor types (columns) with at least 10 samples in each group for each of the 78 cell surface targets (rows). Within each histology, negative values (red) indicate enrichment of the target in metastatic tumors of that histology, while positive values (blue) indicate enrichment of the target in primary tumors. Hierarchical clustering was performed. Green overlaid text indicates Wilcoxon Rank-Sum FDR comparing primary vs. metastatic expression: ≤0.05*, ≤0.01**, ≤0.001***, ≤0.0001***. FIGS. 5A-5C show three portions of a single correlation, with FIG. 5A-5C continuing in order from top to bottom.

FIGS. 6A-6C. Drug repositioning candidates. Boxplots show the metastatic (blue) and primary (gray) cancer expression in the RNAseq data for various cell surface targets. RNA expression levels are percentile rank normalized, ranging from 0 to 1. Green/gray/red dotted line: 1^{st}/50^{th}/90^{th} percentile of housekeeping gene expression.

FIGS. 7A-7D. Selecting a cell line model system. FIG. 7A. Schematic of rationale on identifying optimal cell line based on both cell surface target expression and correlation with high-purity clinical tumor samples of the intended patient population. FIGS. 7B and 7C. Number of cell lines that met the following criteria: 1) cell lines with expression that was ≥95^{th} percentile of expression for each target in the tumor RNA-seq (horizontal blue line) and 2) ≥95^{th} percentile of correlation with tumor samples of each primary/metastatic tumor type (vertical blue line). Gene expression for each cell line was correlated with each high purity primary/metastatic cancer sample, and a median was calculated for each cancer type. Each point represents one cell line targe expression (y-axis) vs. the median correlation of that cell line to a primary or metastatic tumor type (x-axis). Red/green point=cell lines of the same cancer type as the metastatic/primary cancer tissue sample that meet criteria 1+2. Gray dot=cell lines of a different cancer type that meet criteria 1+2. FIG. 7D Number of cell lines (indicated by dot size) that meet criteria 1+2 across tumor types (rows) and targets (columns). Red/green dots represent cell lines that are the same cancer type for primary/metastatic cancer. Gray dots represent cell lines of a different cancer type.

FIGS. 8A-8F. Single cell heterogeneity. FIGS. 8A-8D. Heatmap of the proportion of the tumor cells in each scRNAseq sample that expresses each cell surface target. Rows are targets and columns are scRNAseq samples. Red represents samples with a high proportion of tumor cells expressing each target, green represents samples where a more mixed proportion of tumor cells express each target, and white represents an absence of cell surface target expression. FIGS. 8A-8D constitute four quadrants of a single heatmap, with FIG. 8A constituting the top left, FIG. 8B constituting the top right, FIG. 8C constituting the bottom left, and FIG. 8D constituting the bottom right. FIG. 8E. Percent of cells positive for ERBB2, ERBB3, and TACSTD2 across all cell types and within each cell type subpopulation in replicate lung scRNAseq samples FIG. 8F. Detection of vedotin-conjugate target expression across different pancreatic cell types showing that target expression in alpha cells correlates with presence or absence of hyperglycemia as a clinical toxicity.

FIGS. 9A-9D. Combinations of two targets. FIG. 9A. Logistic regression models were created for every pair of cell surface targets comparing each cancer type and each normal tissue type. Only pairs where 1) expression was higher in cancer vs. normal and the combination had good discriminative power with an F1-score >0.95, and 2) each individual gene was contributing independently, with both having Wald test p-values (corrected for multiple testing)<0.05, were retained. The number of combinations as a function of the proportion of cancer-normal tissue comparisons meeting these two criteria is shown. The red line indicates cell surface target combinations where >90% of the cancer-normal tissue comparisons met criteria 1+2, and is where the examples shown in B are drawn. FIGS. 9B-9D. Example combinations of how gene expression percentiles of two cell surface targets (X and Y axis) can better stratify benign samples (gray) from cancer samples (red).

FIGS. 10A and 10B. Clinical trials. Landscape of cell surface protein targets in clinical trials for cell surface targeted cancer therapies. FIG. 10A. The distribution of targeted cancer therapy types across the trials. FIG. 10B. The number of clinical trials by their current status (as of Oct. 31, 2023).

FIGS. 11A and 11B. RNA-seq sample size. Sample sizes for each normal tissue type (FIG. 11A) and each cancer type (FIG. 11B), divided into primary tumors and metastatic tumors. The sources of these RNA-seq samples are specified, including GTEX, non-GTEX, TCGA, and non-TCGA.

FIG. 12. Microarray sample size. Sample sizes for each cancer type, separated into primary and metastatic tumors.

FIG. 13. High cell surface target RNA expression accurately predicts high protein expression. RNA vs. protein expression in three datasets with matched RNA-seq and mass spectrometry: cancer tissue samples from the Clinical Proteomic Tumor Analysis Consortium (CPTAC), cancer cell lines from the Sanger Cell Model Passports (SCMP), and benign tissues from the Human Protein Atlas (HPA). We defined high expression of protein or RNA expression as being higher than 90% of housekeeping gene protein or RNA expression respectively in each sample, and histograms showing the distribution of accuracies across cell surface targets comparing protein and RNA are shown.

FIGS. 14A-14D. RNAseq expression consistent with known patterns for cell surface targets. Boxplots show the metastatic (blue) and primary (gray) cancer expression in the RNAseq data for various cell surface targets. RNA expression levels are percentile rank normalized, ranging from 0 to 1. Green/gray/red dotted line: 1^{st}/50^{th}/90^{th} percentile of housekeeping gene expression. We benchmarked the percentile expression data to housekeeping gene expression. The 94^{th}, 76^{th}, and 34^{th} percentiles of overall gene expression represented a level ≥90%, ≥50%, and ≥1% of housekeeping genes.

FIGS. 15A-15D. Microarray expression consistent with known patterns for cell surface targets. Boxplots show the metastatic (blue) and primary (gray) cancer expression in the microarray data for various cell surface targets. Expression for each gene is normalized to be the fraction of housekeeping genes with lower expression (e.g. a gene expressed higher than 90% of housekeeping genes in a sample=0.9).

FIGS. 16A-16C. Normal tissue expression. Hierarchical clustering of median bulk RNA expression of 78 cell surface protein targets across 37 normal tissue types. RNA expression levels are percentile normalized, with values ranging from 0 to 1. FIGS. 5A-5C show three portions of a single hierarchical clustering, with FIG. 16A-16C continuing in order from top to bottom.

FIGS. 17A-17D. Examples of primary vs. metastatic dichotomy. Boxplots show the metastatic (blue) and primary (gray) cancer expression in the RNAseq data for various cell surface targets. Two-sided Wilcoxon Rank-Sum test performed.

FIGS. 18A-18C. Drug repositioning candidates—microarray. Boxplots show the metastatic (blue) and primary (gray) cancer expression in the microarray data for various cell surface targets. Expression for each gene is normalized to be the fraction of housekeeping genes with lower expression (e.g. a gene expressed higher than 90% of housekeeping genes in a sample=0.9).

FIG. 19. Single-cell RNAseq distributions. Example distributions of the proportion of the tumor cells in each single-cell RNAseq sample that expresses each cell surface target, grouped by specific cell surface targets and tumor types.

FIG. 20. LLM Generation of a cell surface targeting clinical trials database. The NCI Drug Dictionary definitions and information from ClinicalTrials.gov are used to generate prompts for each model requesting either the gene target or the type of therapy. Gene targets are then combined with the Therapeutic Target Database to create a final list of targets. These targets are then annotated as cell surface or not based on published experimental and computational data. Annotated cell surface targets are then merged with the therapy type results and filtered to return a list containing only those clinical trials for which the model could identify the therapy type as cell surface targeting and the respective gene target. Blue steps indicate the original databases, green steps indicate LLM analysis, purple steps indicate annotation, black step indicate filtering, and yellow steps indicate workflow results.

FIGS. 21A-21H. Model Evaluation and Target Accuracy. FIG. 21A. Workflow for ClinicalTrials.gov intervention labels and NCI Drug Dictionary definitions. Final results of each are concatenated to make the “combined approach” target list. FIG. 21B. Percent of clinical trials correctly labeled as using a cell surface targeting therapy (left) and percent of trials correctly labeled as not utilizing a cell surface targeting therapy (right). Total number of trials in each case is 814. FIG. 21C. Percent of successful matches using the combined approach workflow, categorized by gene targets and trials, for each LLM tested. Total number of targets is 831, total number of trials is 814. FIG. 21D. Percent of successful matches from each workflow for the GPT-4o model, categorized by number of gene targets and number of trials. FIG. 21E. Venn diagram of gene targets successfully matched by the NCI drug dictionary and ClinicalTrials.gov ‘interventions’ workflows using the GPT-4o model. FIG. 21F. Venn diagram of clinical trials successfully matched by the NCI drug dictionary and ClinicalTrials.gov ‘interventions’ workflows using the GPT-4o model. FIG. 21G. Clinical trial matches using the GPT-4o model, categorized by type of therapy investigated and displayed as a percent of total therapy-specific trials. “Other-CAR” includes CAR-DC, CAR-M, and CAR-NK therapies. Numbers at the top of each bar indicate total number of trials for that therapy type. FIG. 21H. Clinical trial matches using the GPT-4o model, categorized by trial phase and displayed as a percent of total trials of each phase. Numbers at the top of each bar indicate total number of unmatched for that phase.

### DETAILED DESCRIPTION OF THE INVENTION

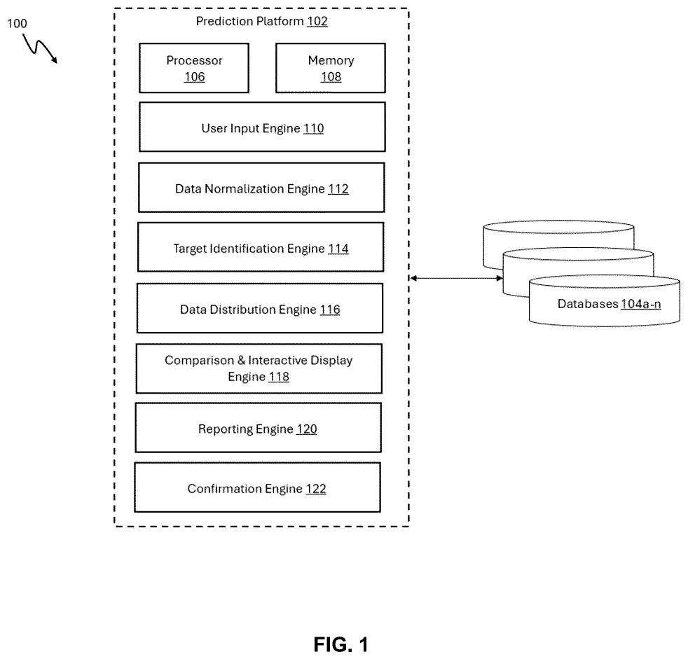

Referring to FIG. 1, a block diagram of a system 100 for generating a prediction model of cell-surface protein expression in cancer is depicted, according to an embodiment. System 100 generally comprises a prediction platform 102 and a plurality of databases 104a-n.

Embodiments described herein include various engines, each of which is constructed, programmed, configured, or otherwise adapted, to autonomously carry out a function or set of functions. The term engine as used herein is defined as a real-world device, component, or arrangement of components implemented using hardware, such as by an application specific integrated circuit (ASIC) or field-programmable gate array (FPGA), for example, or as a combination of hardware and software, such as by a microprocessor system and a set of program instructions that adapt the engine to implement the particular functionality, which (while being executed) transform the microprocessor system into a special-purpose device. An engine can also be implemented as a combination of the two, with certain functions facilitated by hardware alone, and other functions facilitated by a combination of hardware and software. In certain implementations, at least a portion, and in some cases, all, of an engine can be executed on the processor(s) of one or more computing platforms that are made up of hardware (e.g., one or more processors, data storage devices such as memory or drive storage, input/output facilities such as network interface devices, video devices, keyboard, mouse or touchscreen devices, etc.) that execute an operating system, system programs, and application programs, while also implementing the engine using multitasking, multithreading, distributed (e.g., cluster, peer-peer, cloud, etc.) processing where appropriate, or other such techniques.

An (e.g., each) engine can be realized in a variety of physically realizable configurations. For example, an engine can itself be composed of sub-engines, each of which can be regarded as an engine in its own right. Moreover, in the embodiments described herein, each of the various engines corresponds to a defined functionality; however, it should be understood that in other contemplated embodiments, each functionality can be distributed to more than one engine. Likewise, in other contemplated embodiments, multiple defined functionalities can be implemented by a single engine that performs those multiple functions, possibly alongside other functions, or distributed differently among a set of engines than specifically illustrated in the examples herein. In one example, the engines described herein are executed by a processor (e.g. an ASIC) according to instructions stored on memory operably coupled to the processor. In embodiments, system 100 can be a cloud-based service such that execution of associated workflows can be distributed across a network of multiple computing devices (e.g., with each device having its own processor and memory).

Prediction platform 102 generally comprises a processor 106, a memory 108, a user input engine 110, a data normalization engine 112, a target identification engine 114, a data distribution engine 116, a comparison and interactive display engine 118, a reporting engine 120, and a confirmation engine 122.

Processor 106 comprises one or more digital signal processors (DSPs), microprocessors, application specific integrated circuits (ASICs), field programmable logic arrays (FPGAs), or other equivalent integrated or discrete logic circuitry. Accordingly, the term “processor” as used herein can refer to any of the foregoing structures or any other physical structure suitable for implementation of the described functions or techniques of the various engines.

Memory 108 comprises non-transitory computer-readable media, which corresponds to a tangible medium such as a data storage media (e.g., RAM, ROM, EEPROM, flash memory, or any other medium that can be used to store desired program code in the form of instructions or data structures which can be accessed by a computer). In one aspect, instructions or code can be stored on memory 108 and executed by processor 106.

User input engine 110 comprises a set of instructions to provide a graphical user interface to a user (or user device) to access prediction platform 102. For example, user input engine 110 can comprise a user interface on a user device, such as computer operable by a user including a desktop computer, laptop computer, tablet, mobile computing device, server, workstation, Internet-of-things device, or other computing device. In one aspect, a user can input clinical or research-based whole-transcriptome RNA-sequence (RNA-seq) data via the user interface provided by user input engine 110.

In one aspect, user input engine 110 can conduct pre-processing of the inputted data. For example, user input engine 110 can conduct pre-processing to generate gene expression levels. In one aspect, pre-processing can include initial normalization for both gene length and sequencing depth (e.g. calculating Transcripts Per Kilobase Million (TPM), Fragments Per Kilobase Million (FPKM), Reads Per Kilobase Million (RPKM), etc.). Accordingly, user input engine 110 can output a pre-processed matrix of expression levels of genes within each sample (e.g. gene expression matrix).

A gene expression matrix comprises a table where genes are rows and samples (or cells) are columns, with each cell containing the measured expression level of a specific gene in a specific sample. Such matrices can be used as foundational input for downstream analyses. As illustrated, the expression levels are typically quantified as read counts, FPKM, or TPM, depending on the experimental protocol and suitable normalization method(s).

Data normalization engine 112 comprises a set of instructions to normalize data from user input engine 110. In one aspect, data normalization engine 112 receives as input the pre-processed (normalized) RNA-seq gene expression data from user input engine 110 and conducts rank and percentile normalization. For example, data normalization engine 112 determines an ordinal rank order of all the genes within each sample. In one aspect, ordinal rank can be calculated by the function RANK 0 in the R programming language, which assigns ranks to elements within a vector (e.g. sorting from smallest to greatest and assigning a rank based on that sorting). RANK 0 returns a vector of the same length as the input, where each element represents the rank of the corresponding value in the original vector. In a further example, data normalization engine 112 then scales the ranks to be a percentile score between 0 and 1 within each sample. In one aspect, percent score (e.g. percent scaling) can be calculated by dividing each rank by the highest rank. Accordingly, data normalization engine 112 can output a matrix data structure of percentile levels of genes within samples. In one aspect, the matrix data structure comprises an M×N matrix of rows as samples and columns as genes, with the ranked and scaled data within respective cells.

In a technical improvement to data storage, the matrix data structure implements more efficient storage and data retrieval in memory. In particular, in contrast to traditional methods in which a relative rank would be stored in separate data structure and a gene expression matrix stored in a separate data structure with relational links between thereby increasing the number of storage reads and requiring separate logic to connect the relationships between the data, the instant matrix does not require relational links, but rather stores the rank data within its unique matrix data structure. The unique matrix data structure storage in RAM allows for fast row and column-level computation, making the calculation of ranks and percentiles faster than traditional methods.

In a specific technical improvement provided by the aforementioned matrix data structure, the structure be stored in a contiguous memory layout. In one example, the matrix in RAM (for example, in C, C++, or Fortran) can be stored as a contiguous block of memory (row-major or column-major order), which means all elements are laid out in a single stretch of addresses, like: A[0][0],A[0][1],A[0][2], . . . , A[1][0],A[1][1], . . . . Because of this, by knowing the base address and the row/column sizes, the address of any element can be computed with a simple arithmetic formula:

$$Address ( A [ i ] [ j ] ) = BaseAddress + ( i \times NumCols + j ) \times ElementSize$$

Such addressing results in no pointer chasing and no indirection, but rather, just arithmetic and direct memory access.

In another specific technical improvement provided by the aforementioned matrix data structure, embodiments take advantage of cache locality. In one example, CPUs often do not fetch single elements from RAM, but rather, CPUs fetch chunks of cache lines (commonly 64 bytes). Since the matrix data is contiguous, accessing one element often brings in its neighbors too. By looping through rows or columns in order, the next elements are already waiting in cache. Such spatial locality makes access feel “fast,” since most loads come from the CPU cache, not main RAM.

In another specific technical improvement provided by the aforementioned matrix data structure, embodiments take advantage of prefetching. Modern CPUs detect access patterns (like sequential traversal) and automatically issue prefetches to bring in the next cache lines before the program actually requests them. Accordingly, when embodiments loop for A[i][j+1], such data is often already sitting in cache.

In another specific technical improvement provided by the aforementioned matrix data structure, embodiments implement low overhead addressing. For example, matrix data structure stored in contiguous memory, indexing is just: a multiply and an add, which is a single arithmetic calculation for the offset. Compared that to pointer-based structures (e.g., linked lists), where each access requires dereferencing a pointer in RAM, which can be scattered across memory and cache-unfriendly, and further complicated by relational database links.

Further, by the improved (efficient) data structure minimizing the data stored and being stored in an efficient transfer structure (e.g. contiguous chunks), the amount and type of data needed to be transmitted to networked data storage (e.g. to databases 104) is reduced, thereby improving network congestion.

The reason this works is because rank/percentile normalization allows for a comparison across technologies and platforms that can give very different quantitative values. For example, in the simplest case, one assay could indicate that Gene A is 1000 and Gene B is 10000, and a different assay could indicate that Gene A is 10 and Gene B is 20, even in the same exact sample. However, the relative relationship and therefore the rank and percentile is preserved as long as the assay accurately identifies that Gene B is >Gene A.

Further, in a specific technical improvement to computerized data analysis, the percentile normalization allows some degree of missing data, as long as the missing data is somewhat evenly distributed throughout. For example, with 10000 genes ranked 1-10000, the percentile ranks will be 1/10000 to 10000/10000. If in a particular sample or dataset, the even ranks were all missing (which would be an enormous rate of missing data unlikely to be present in the real world), then the percentile ranks of the remaining genes would actually be very similar, especially at the higher ranks. The lowest rank would be 1/5000, which is numerically quite close to 1/10000, differing by only 1/10000. The highest rank would be identical at 1, with lower differences the higher the rank. In particular, computerized data analysis robustness is improved over traditional methods. For example, missing data often leads to undefined operations (e.g., division by zero, NaNs in matrix multiplications). The percentile normalization that accounts for missing data ensures the system can still compute valid values (e.g., averaging only over available features, imputing normalized scales). Accordingly, the specific technical improvements include preventing computational failures and allowing models to process incomplete datasets without crashing.

In one aspect, data normalization engine 112 is further configured to retrieve RNA-seq data from one or more databases 104a-n. As described above with respect to RNA-seq data from user input, data normalization engine 112 can conduct rank and percentile normalization and determine an ordinal rank order of all the genes within each sample for sample data from one or more databases 104a-n.

Target identification engine 114 comprises a set of instructions to receive as input the user-inputted, then ranked and percentile-normalized per-sample data from data normalization engine 112 as well as clinical cell surface targets from external databases 104 (e.g. ranked and percentile-normalized per-sample by data normalization engine 112), and subsets the gene expression data to just the cell surface targets (CSTs). For example, in one aspect, target identification engine 114 matches gene names and outputs an array of percentile levels of cell surface target genes only within each sample.

Obtaining a list of clinical cell surface targets is traditionally not straightforward. Typically, this involved manually searching through clinical trial descriptions. Accordingly, embodiments described herein solve the problem of identifying CST targes by a technical solution using large language models (LLMs). An example using LLMs to identify CST trials and extract the targets of those trials is provided in the following examples. The resulting list is the list of clinical cell surface targets.

Data distribution engine 116 comprises a set of instructions to receive as input the CSTs from target identification engine 114. In one aspect, using the CST data, data distribution engine 116 generates one or more histograms. For example, data distribution engine 116 can implement ggplot for the R statistical programming language. In one aspect, a distribution (histogram) of tumors is created for each CST including each tissue, cancer type, and subtype. In one aspect, a distribution (histogram) of benign tissue is created for each CST including each tissue, type, and subtype.

Comparison and interactive display engine 118 comprises a set of instructions to receive as input, the distribution of cancer/normal rank/percentile normalized CST expression from data distribution engine 116, and an individual's rank/percentile normalized CST expression from target identification engine 114. Comparison and interactive display engine 118 is further configured to identify a percentile for each CST in an individual patient compared to expression across tumors and normal tissues. In one aspect, for a given CST, the rank/percentile in an individual patient is compared to the distribution of rank/percentiles across tumors and normal tissue. This comparison is then assigned a percentile. For example, if for Gene A in an individual patient has a rank/percentile score >70% of tumor samples across all tumor types, then this would represent the 70th percentile. This can also return more granular information as well, including the percentile across specific tumor or normal tissue types/subtypes. Comparison and interactive display engine 118 is accordingly configured to output the percentile for each CST in a specific patient compared to the overall distribution of tumors/normal tissues.

Again using the concept of rank/percentile normalization allowing for a comparison across technologies and platforms that can give very different quantitative values, embodiments thereby utilize the relative relationship and therefore the rank and percentile that is preserved between genes as long as the assay accurately identifies that Gene B is >Gene A. In one aspect, a user interface can be provided to a user device that includes a graphical representation of the percentiles for each CST in a specific patient compared to the overall distribution of tumors/normal tissues.

In a technical improvement to user interfaces, an image is pre-generated for every possible integer percentile with such information built into the filename, so that the slider simulates being dynamic by pulling in a different image by filename. In this way, the user interface is pseudo-dynamic. The file lookup index is encoded in the filename to be utilized based on the user input; for example, “Genename_inputsamplepercentile_pancanpercentile.png.” When the user inputs a specific gene and input sample percentile, embodiments locate the exact filename based on the gene and percentile input for subsequent user interface population.

In one aspect, in which the user interface comprises a website, the website design is greatly simplified over existing processing-intensive displays. Accordingly, instead of traditional interfaces in which the user interface is re-created with every user interaction, embodiments are more efficient in data recall because the computing-intensive work of creating visualizations is pre-generated and only done once, and the less-computing-intensive work of recalling an existing file is conducted instead. Moreover, the unique indexing by file name actually removes traditional lookup index data structures, which would normally be used to connect user input to a file to be recalled. This improves computing processor usage as well as data storage.

Reporting engine 120 comprises a set of instructions to receive as input individual patient CST expression compared to tumor/normal distribution from comparison and interactive display engine 118. Reporting engine 120 is further configured to identify one or more CSTs from an individual patient that have high relative CST expression and low normal tissue CST expression compared to the distributions. In one aspect, the user can specify thresholds for both tumor and normal tissue. For example, a user could ask for CSTs where the specific patient's CST expression is >80th percentile than the pan-cancer distribution, and <50th percentile than the normal tissue distribution. Furthermore, this can be further refined to specify specific cancer types/subtypes or normal tissue types. For example, perhaps the specific patient has metastatic prostate cancer, so a user could compare only against metastatic prostate cancer. Or perhaps the patient has poor kidney function, so a user could ensure the expression in the kidney is particularly low. In one aspect, reporting engine 120 outputs a list of top CSTs.

Confirmation engine 122 can instruct a confirmation with immunohistochemistry (or some other similar technique) to be performed on the patient's tumor tissue (e.g. a laboratory test) based on results from the outputted list of reporting engine 120.

Databases 104a-n each respectively comprise a data storage device accessible (e.g. read, write) by prediction platform 102 (e.g. data normalization engine 112, in one aspect). Databases 104 can be implemented according to a general-purpose database management storage system (DBMS) or relational DBMS as implemented by, for example, ORACLE, IBM DB2, Microsoft SQL Server, PostgreSQL, MySQL, SQLite, LINUX, or UNIX solutions.

In one example, databases 104 each comprise a different type of RNA-sequence-associated data, such as a first database 104a comprising benign tissue data, a second database 104b comprising primary tumor data, and a third database 104c comprising metastatic tumor data. In one aspect, databases 104a-c comprise data from a high proportion. In aspects, a high proportion means greater than 20%, greater than 23.5%, greater than 30%, greater than 35%, greater than 45%, or greater than 50% of patients with metastatic tumors rather than primary tumors, as cell surface therapies are typically targeted towards the former rather than the latter. Additional data can also be added, and because of the flexibility of the above framework of system 100, seamlessly integrated. Any gene expression dataset can be rank-ordered and a percentile calculated with ease, which allows the gene expression data to be integrated in similar data in system 100. In one aspect, a databases 104 can comprise data storage for the engines of prediction platform 102, such as the storage of the matrix data structure(s).

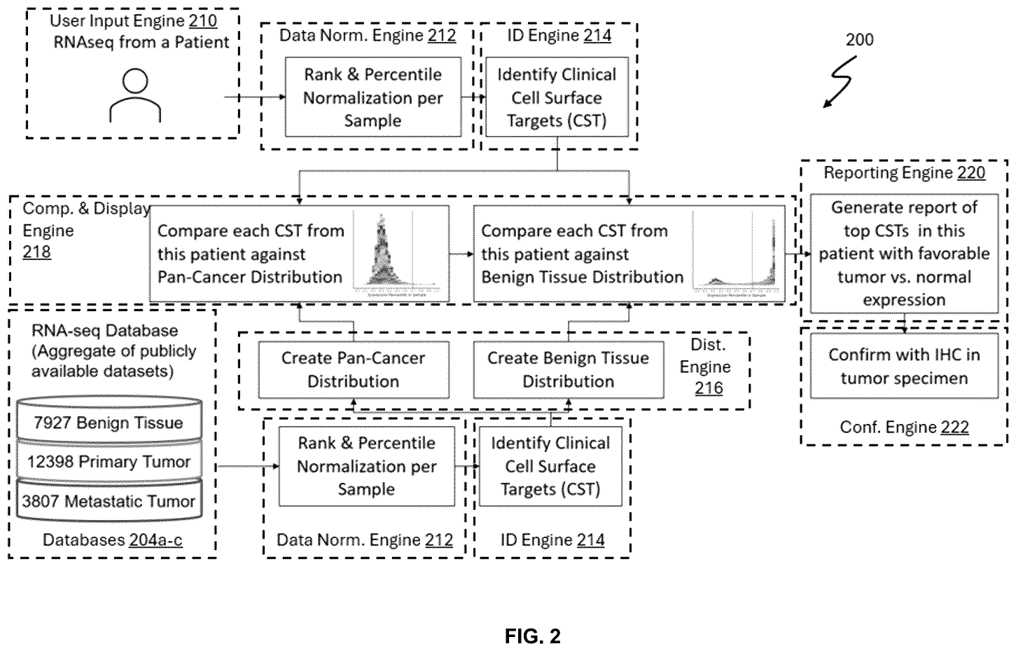

Referring to FIG. 2, a functional block diagram of a system 200 for generating a prediction model of cell-surface protein expression in cancer is depicted, according to an embodiment. In FIG. 2, respective engines are depicted in dashed line relative to system flow operations. In an embodiment, the components of system 200 correspond to the components of system 100, but which are renumbered here but not re-described for conciseness. For example, while not explicitly depicted, system 200 can comprise a prediction platform including one or more processors and operably coupled memory. System 200 further comprises databases 204a-c, which can correspond to databases 104 in system 100. Further, the prediction platform of system 200 can comprise a user input engine 210 (corresponding to user input engine 110), a data normalization engine 212 (corresponding to data normalization engine 112), a target identification engine 214 (corresponding to target identification engine 114), a data distribution engine 216 (corresponding to data distribution engine 116), a comparison and interactive display engine 218 (corresponding to comparison and interactive display engine 118), a reporting engine 220 (corresponding to reporting engine 120), and a confirmation engine 222 (corresponding to confirmation engine 122).

In the workflow of system 200, RNA-seq for a patient is received by user input engine 210. Optionally (not depicted, user input engine 210 can conduct pre-processing (e.g. initial normalization) on the received RNA-seq. Rank and percent normalization per sample is conducted by data normalization engine 212. Next, clinical cell surface targets are identified by target identification engine 214 using the RNA-seq.

Referring now to databases 204a-c, three separate databases are depicted. As illustrated, databases 204a-c comprises a first database comprising benign tissue data (7927), a second database comprising primary tumor data (12398), and a third database comprising metastatic tumor data (3807). In one aspect, the data in databases 204 is an aggregation of publicly available datasets. Using the data from at least one of databases 204a-c, rank and percentage normalization per sample is conducted by data normalization engine 212. Next, clinical cell surface targets are identified by target identification engine 214 using the normalized data.

Data distribution engine 216 then creates a pan cancer distribution using the CST targets identified by target identification engine 214. Optionally, data distribution engine 216 can create a benign tissue distribution using the CST targets identified by target identification engine 214. Comparison and interactive display engine 218 then compares each CST from the patient RNA-seq data (via input engine 210) to the pan-cancer distribution, and further compares each CST from the patient RNA-seq data (via input engine 210) to the benign tissue distribution.

A report of top CSTs is generated for the patient by reporting engine 220. In one aspect, the report can include favorable tumor compared to normal expression.

Finally, a confirmation with immunohistochemistry can be performed (e.g. instructed) on the patient's tumor tissue based on results from reporting engine 220.

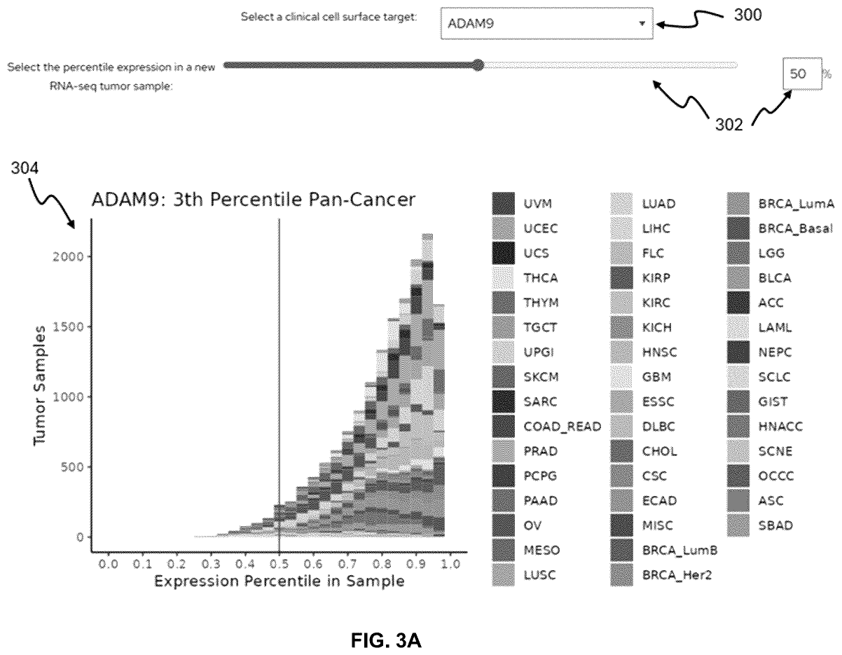

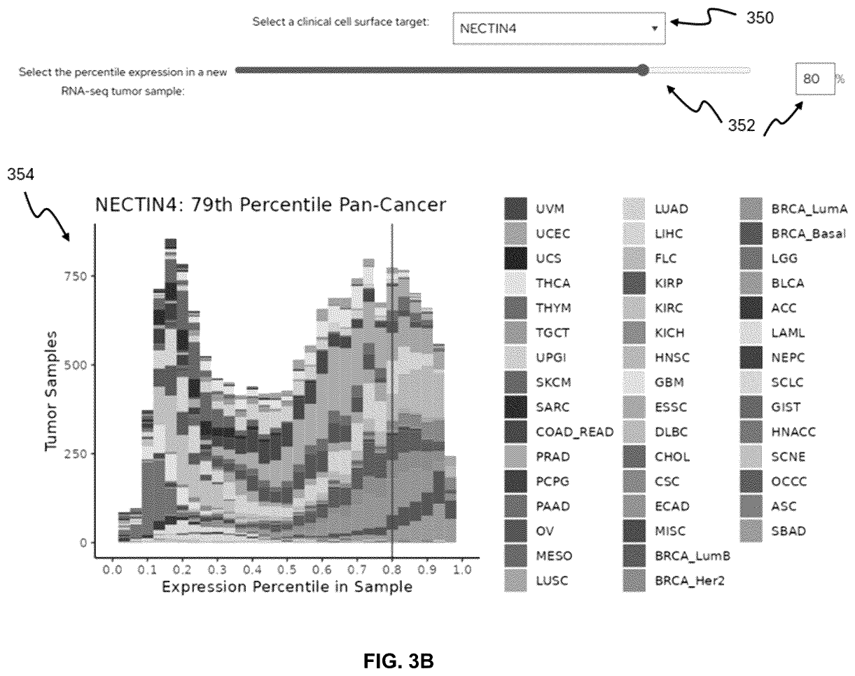

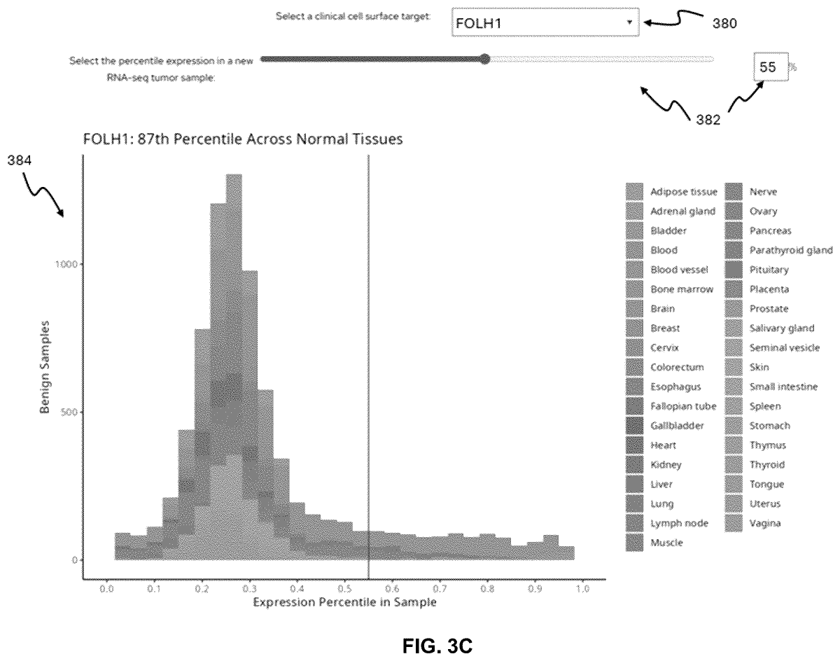

Referring to FIGS. 3A-3B, screenshots of graphical user interfaces presented by systems 100, 200 are depicted, according to embodiments. For example, comparison and interactive display engine 118, 218 can generate and present such graphical user interfaces.

In FIG. 3A, a graphical user interface comprises a plurality of display elements including an interactive component 300 as a drop-down menu to select a clinical cell surface target (e.g. ADAM9 in FIG. 3A), an interactive component 302 as a slider or text field to select a percentile of expression in a new RNA-seq tumor sample (e.g. 50% in FIG. 3A, which means that the expression of the selected clinical cell surface target was expressed higher than 50% of all genes in the new RNA-seq tumor sample), and a graphical display 304 of an expression of the percentile of the sample against a pan-cancer distribution of many tumor samples. Individual colors (e.g. in graphical display 304 and the adjacent key) represent tumor types or subtypes, and can further be divided into primary vs. metastatic or other subgroups. The percentile expression of the selected clinical cell surface target of the new RNA-seq sample can be compared against this pan-cancer distribution to identify the percentile within this distribution. A high percentile indicates that the expression of the selected clinical cell surface target of the new RNA-seq sample is high compared to many other tumors, and therefore potentially a good target. This same method can be applied to subsets of tumors relevant to the clinical case (e.g. look at prostate cancer tumors only if the new RNA-seq sample is also prostate cancer).

In FIG. 3B, a graphical user interface comprises a plurality of display elements including an interactive component 350 as a drop-down menu to select a clinical cell surface target (e.g. NECTIN4 in FIG. 3B), an interactive component 352 as a slider or text field to select a percentile of expression in a new RNA-seq tumor sample (e.g. 80% in FIG. 3B), and a graphical display 354 of an expression of the percentile of the sample against a pan-cancer distribution of many tumor samples as above.

In FIG. 3C, a graphical user interface comprises a plurality of display elements for normal tissue including an interactive component 380 as a drop-down menu to select a clinical cell surface target (e.g. FOLH1 in FIG. 3C), an interactive component 382 as a slider or text field to select a percentile of expression in a new RNA-seq tumor sample (e.g. 55% in FIG. 3C), and a graphical display 384 of an expression of the percentile of the sample against a distribution of many normal tissue samples. The percentile expression of the selected clinical cell surface target of the new RNA-seq sample can be compared against this normal tissue distribution to identify the percentile within this distribution. A high percentile indicates that the expression of the selected clinical cell surface target of the new RNA-seq sample is high relative to normal tissues, and therefore potentially a good target.

Some aspects of the invention are directed to methods of generating a prediction model of cell-surface protein expression, such as cell-surface protein expression in cancer.

The methods can comprise a step of determining a gene expression profile for each training cell sample in a set of training cell samples.

“Set” as used herein refers to a finite collection of one (e.g., non-empty) or more members.

The term “training” appended to any element described herein refers to the element being employed or to generate a prediction model of the invention. Examples include cell samples (e.g., training cell sample, training cancer cell sample, training primary tumor cell sample, training metastatic tumor cell sample), genes (gene training gene) and rankings (training ranking).

“Cell sample” as used herein refers to a sample comprising one or more cells or processed forms thereof. In some versions, a given cell sample can comprise a single cell or a processed form thereof. In some versions a given cell sample can comprise multiple cells (e.g., such as a tissue sample) or a processed form thereof. The processing of the cells can comprise any methods suitable for preparing specific content of the original cells for downstream analysis and can comprise one or more lysis and/or purification steps. In some versions, the specific content can comprise mRNA. In some versions, the downstream analysis can comprise determining gene expression profiles.

In some versions, the training cell samples comprise primary cell samples. Primary cell samples are samples comprising one or more primary cells or processed forms thereof. Primary cells are cells that have been removed from a live donor/specimen and have been established in an in vitro environment. In some versions, the training cell samples comprise secondary cell samples. Secondary cell samples are samples comprising one or more secondary cells or processed forms thereof. Secondary cell samples are cells that have been immortalized and can divide indefinitely. Secondary cell samples and secondary cells are also referred to herein as “cell line samples” and “cell lines,” respectively.

In some versions, the training cell samples comprise cell samples from one or more organs. Exemplary organs include heart, blood (blood is considered an organ herein), esophagus, stomach, liver, gallbladder, pancreas, intestines, colon, mesentery, rectum, anus, hypothalamus, pituitary gland, pineal body or pineal gland, thyroid, adrenal gland, kidney, ureter, bladder, and urethra, lymph, lymph nodes, vessels, skin, muscle, brain, spinal cord, ovary, oviduct, uterus, vulva, vagina, testicle, vas deferens, seminal vesicle, prostate, penis, pharynx, larynx, trachea, bronchi, lungs, diaphragm, bone, cartilage, ligament, and tendon.

In some versions, the training cell samples comprise cell samples comprising one or more specific cell types. “Cell type” refers to a type of cell identifiable by one or more markers. Exemplary types of cells include alpha cells, beta cells, T cells, macrophages, etc.

In some versions, the training cell samples comprise cancer cell samples. Cancer cell samples are samples comprising one or more cancer cells or processed forms thereof. In some versions, the cancer cell samples comprise primary tumor cell samples. Primary tumor cell samples are samples comprising one or more primary tumor cells or processed forms thereof. Primary tumor cells are primary cells isolated from a primary tumor. Primary tumors are original, or first, tumors appearing in a body. In some versions, the cancer cell samples comprise metastatic tumor cell samples. Metastatic tumor cell samples are samples comprising one or more metastatic tumor cells or processed forms thereof. Metastatic tumor cells are cancer cells that have separated from a primary tumor and spread to other parts of the body. In some cases, the metastatic tumor cells form new tumors, referred to as metastatic tumors or secondary tumors. In some versions, the cancer cell samples comprise primary tumor cell samples and metastatic tumor cell samples.

In some versions, the training cell samples comprise one or more types of cancer cell samples. A type of cancer cell sample is a sample comprising a particular type of cancer cell. Types of cancer cells can be defined, for example, by one or more markers (genetic, protein, etc.), one or more organ origins, one or more tissue origins, one or more cell origins, or any combination thereof. Exemplary types of cancers that can define a particular type of cancer cell include adrenocortical carcinoma, anal squamous cell carcinoma, bladder urothelial carcinoma, breast invasive carcinoma (basal subtype), breast invasive carcinoma (HER2 subtype), breast invasive carcinoma (luminal A subtype), breast invasive carcinoma (luminal B subtype), cholangiocarcinoma, colorectal adenocarcinoma, cervical squamous cell carcinoma, cancer of unknown primary, diffuse large B-cell lymphoma, endocervical adenocarcinoma, esophagus squamous cell carcinoma, fibrolamellar carcinoma, glioblastoma multiforme, gastrointestinal stromal tumor, adenoid cystic carcinoma, head and neck squamous cell carcinoma, kidney chromophobe, kidney renal clear cell carcinoma, kidney renal papillary cell carcinoma, acute myeloid leukemia, brain lower grade glioma, liver hepatocellular carcinoma, lung adenocarcinoma, lung squamous cell carcinoma, mesothelioma, miscellaneous, prostate neuroendocrine carcinoma, clear cell ovarian cancer, ovarian serous cystadenocarcinoma, pancreatic adenocarcinoma, pheochromocytoma and paraganglioma, prostate adenocarcinoma, sarcoma, small bowel adenocarcinoma, small cell lung cancer, other small cell or neuroendocrine tumor, skin cutaneous melanoma, testicular germ cell tumor, thyroid carcinoma, thymoma, uterine corpus endometrial carcinoma, uterine carcinosarcoma, esophagus/stomach adenocarcinoma, and uveal melanoma. In various versions of the invention, the training cell samples can include at least 1, at least 5, at least 10, at least 15, at least 20, at least 25, at least 30, at least 35, at least 40, at least 45, or each of the aforementioned types, in any combination.

In some versions, the training cell samples comprise non-cancer samples (samples of one or more non-cancer cells or processed forms thereof). In some versions, the training cell samples comprise cancer cell samples and non-cancer cell samples.

“Gene expression profile” as used herein refers to a set of gene-expression values for a set of gene-expression-profile genes present in a given cell sample. The gene-expression values can be in the form of a quantitation of the mRNA levels of various genes within a given cell sample. The gene-expression values can be an absolute quantitation of mRNA levels (e.g., raw numbers of measured mRNA copies) or a relative quantitation comprising normalized and/or analytically processed values. Determining the gene expression profiles can comprise measuring empirically determining the gene-expression values (e.g., measuring the number of mRNA copies) and/or, depending on the availability of data for a particular cell sample, downloading the gene expression profiles from a database, among other methods. Methods for empirically determining gene-expression values are well known in the art and include RNA-seq and gene expression microarrays, among other methods. See, e.g., Stark et al. 2019 (Stark R, Grzelak M, Hadfield J. RNA sequencing: the teenage years. Nat Rev Genet. 2019 November; 20(11):631-656).

The set of gene-expression-profile genes in a given gene expression profile will depend on the method employed to determine the gene expression profile. The gene-expression-profile genes represented in gene expression profiles determined using RNA-seq, for example, can typically encompass all, or nearly all, of the genes present in a cell sample. The gene-expression-profile genes represented in gene expression profiles determined using a gene expression microarray, by contrast, will encompass only those genes represented on the array. Accordingly, depending on the method(s) employed to determine the gene expression profiles, the various sets of gene-expression-profile genes in the gene expression profiles in any given embodiment of the invention may be the same or different.

In various embodiments of the invention, the set of gene-expression-profile genes in at least one, some, or each of the gene expression profiles comprises at least 500 genes, at least 1,000 genes, at least 2,500 genes, at least 5,000 genes, at least 7,500 genes, at least 10,000 genes, at least 12,500 genes, at least 15,000 genes, at least 17,500 genes, or at least 19,000 genes.

The sets of gene-expression-profile genes in the gene expression profiles of the invention preferably comprise one or more cell-surface protein genes. “Cell-surface protein gene” refers to a gene that expresses a protein that can be present on a surface of a cell. The presence of the protein a surface of a cell can be constitutive, non-constitutive, aberrant, or otherwise. Exemplary cell-surface protein genes of the invention include FOLH1, MUC1, MAGEA3, NCAM1, CD276, EPCAM, GPNMB, ERBB2, CEACAM5, CD70, CA6, SLC44A4, TNC, MSLN, TNFRSF8, EGFR, GUCY2C, FOLR1, TACSTD2, TACSTD2, ENPP3, MET, DLL3, SLC39A6, F3, EFNA4, NECTIN4, KIT, GPA33, FGFR3, PROM1, LRRC15, ROR1, PSCA, HAVCR1, TFRC, GPC3, CDH6, ERBB3, AXL, ALCAM, PTK7, SLC34A2, SSTR2, LY75, ROR2, MUC16, CD46, ISG20, IGF1R, GRPR, ROBO1, TNFRSF10B, TNFRSF9, STEAP1, CLDN18, ITGB6, ICAM1, TPBG, TYRP1, CD22, CD274, HLA-G, CD83, CLDN6, VTCN1, FAP, FLT1, CXCR4, EPHA2, CA9, EPHA5, PMEL, TM4SF1, ADAM9, CD38, FN1, CEACAM6, and NCR2. In various versions of the invention, at least one, some, or each of the sets of gene-expression-profile genes of the invention can include at least 1, at least 5, at least 10, at least 15, at least 20, at least 25, at least 30, at least 35, at least 40, at least 45, or each of the aforementioned cell-surface protein genes, in any combination.

The sets of gene-expression-profile genes in the gene expression profiles of the invention preferably comprise one or more housekeeping genes. The term “housekeeping gene” is well known in the art. Housekeeping genes are generally understood to be constitutive genes that are required for the maintenance of basic cellular function and are expressed in all cells of an organism under normal and pathophysiological conditions.

The methods of the invention can further comprise a step of determining a set of training genes from the gene-expression-profile genes. In preferred versions, the set of training genes comprises a common set of gene-expression-profile genes represented in each of the gene expression profiles. The set of training genes can accordingly be determined by determining a common set of gene-expression-profile genes represented in each of the gene expression profiles.

In various versions of the invention, the set of training genes can comprise at least 500 genes, at least 1,000 genes, at least 2,500 genes, at least 5,000 genes, at least 7,500 genes, at least 10,000 genes, at least 12,500 genes, at least 15,000 genes, at least 17,500 genes, or at least 19,000 genes.

In preferred versions, the set of training genes comprises a set of cell-surface protein genes. In various versions of the invention, the set of cell-surface protein genes in the set of training genes can comprise at least 1, at least 5, at least 10, at least 15, at least 20, at least 25, at least 30, at least 35, at least 40, at least 45, at least 50, at least 55, at least 60, at least 65, at least 70, or each of FOLH1, MUC1, MAGEA3, NCAM1, CD276, EPCAM, GPNMB, ERBB2, CEACAM5, CD70, CA6, SLC44A4, TNC, MSLN, TNFRSF8, EGFR, GUCY2C, FOLR1, TACSTD2, TACSTD2, ENPP3, MET, DLL3, SLC39A6, F3, EFNA4, NECTIN4, KIT, GPA33, FGFR3, PROM1, LRRC15, ROR1, PSCA, HAVCR1, TFRC, GPC3, CDH6, ERBB3, AXL, ALCAM, PTK7, SLC34A2, SSTR2, LY75, ROR2, MUC16, CD46, ISG20, IGF1R, GRPR, ROBO1, TNFRSF10B, TNFRSF9, STEAP1, CLDN18, ITGB6, ICAM1, TPBG, TYRP1, CD22, CD274, HLA-G, CD83, CLDN6, VTCN1, FAP, FLT1, CXCR4, EPHA2, CA9, EPHA5, PMEL, TM4SF1, ADAM9, CD38, FN1, CEACAM6, and NCR2, in any combination.

In preferred versions, the set of training genes comprises one or more housekeeping genes.

The methods of the invention can further comprise a step of ranking the gene-expression values for all of the training genes in each gene expression profile to thereby obtain a training ranking for each gene expression profile. Each training ranking comprises a rank for the gene-expression value for each training gene within the gene expression profile. “Ranking” as used herein refers to ascribing a value (a “rank”) to each gene-expression value that indicates whether a given gene has a higher gene-expression value, a lower gene expression value, or an equal gene expression value with respect to each of the other training genes in a given gene expression profile.

In some versions of the invention, the ranking is an ordinal ranking comprising an ordinal rank for the gene-expression value for each training gene in the gene expression profile. In an ordinal ranking, items (e.g., gene-expression values) are ranked/ordered, and are only able to be classified as higher or lower than the other items in the set such that there is no indication in the final ranking of the degree/amount of distance between items. The ranking in some embodiments of the invention accordingly comprise ordinally ranking the gene-expression values for all of the training genes in each gene expression profile to thereby obtain an ordinal training ranking for each gene expression profile. In some versions, each gene-expression value is assigned an integer representing the value's position in the ranking.

In some versions of the invention, the ranking comprises determining a quantile ranking for each gene-expression value in each gene expression profile. The quantile ranking can comprise determining a number of genes in the set of training genes and dividing each ordinal rank by the number of genes in the set of training genes. Some embodiments of the invention accordingly comprise transforming each ordinal ranking into a quantile ranking by dividing the ordinal rank for the gene-expression value for each gene within the gene expression profile by the number of genes in the set of training genes. This process can thereby provide a quantile rank for each cell-surface protein gene in each training cancer cell sample in the set of training cell samples.

The methods of the invention can further comprise a step of identifying, for each training gene, the training cell samples having a same rank. This identification can serve to generate, for at least one, some, or all the training genes (such as at least one, some, or each of the training cell-surface protein genes), a distribution of the training cell samples or various types or subtypes of training samples having each possible rank. The distribution can serve to compare cancer cells to non-cancer cells, various types of cancer cells to other various types of cancer cells, cancer cells to cell lines, primary cancer cells to metastatic cancer cells, types of cancer cells currently treated with a cell-surface targeting agent to cancer cells not currently treated with a cell-surface targeting agent, various single cells to various other single cells, etc., to determine potential cell-surface targets in one or more of such cells.

In some versions, for example, the models of the invention can bused to determine a predicted cell-surface target on a patient cancer cell sample. The methods can comprise: ranking gene-expression values of all of the training genes in the patient cancer cell sample to obtain a patient ranking; selecting one or more training genes having a threshold rank in the patient ranking to thereby obtain one or more patient genes; comparing the rank(s) of the one or more patient genes in the patient cancer cell sample with the rank(s) of the one or more patient genes in the training cell samples to determine a number, type, and/or proportion of training cancer cell samples having the same rank(s) for the one or more patient genes; optionally, empirically testing cell-surface expression of the one or more patient genes in a test cancer cell sample from the patient; and, optionally, treating the patient with a therapeutic targeting surface expression of any of the one or more of the patient genes confirmed to be expressed in the test cancer cell sample from the patient.

The step of determining the patient ranking can be performed by determining a gene expression profile for the patient cell sample and ranking the gene-expression values of the training genes in the gene expression profile as described elsewhere herein for the training cell samples.

The threshold rank in the step of selecting one or more training genes having the threshold rank in the patient ranking to obtain one or more patient genes can be any user-defined threshold rank. In some versions, the user-defined threshold rank can be based on a user-defined percentage of housekeeping genes having a lower rank, such as 50%, 55%, 60%, 65%, 70%, 75%, 80%, 85%, 90%, 95%, or 99% of housekeeping genes having lower rank.

The comparing step can be used to determine a probability of the patient genes having a given rank being present in a cancer cell versus a non-cancer cell, being present in various types of cancer cells versus other various types of cancer cells, being present in primary cancer cells versus metastatic cancer cells, being present in metastatic cancer cells versus primary cancer cells, etc.

Once patient genes with particular characteristics are determined in the comparing step, the patient genes with such characteristics can be empirically tested for cell-surface expression in the test cancer cell sample from the patient. The cell-surface expression can be tested with immunohistochemistry, flow cytometry, cell culture and immunofluorescence staining, or any other method suitable for detecting the presence of a protein on a surface of a cell.

If one or more of the patient genes are confirmed to be expressed on the surface of the test cancer cell, the patient can then be treated with a therapeutic targeting surface expression of that gene.

In some versions, the prediction models of the invention can be used to identify a cancer type expressing a cell-surface protein. The methods can comprise: determining the ranks of one of the cell-surface protein genes across the training cancer cell samples of different types; selecting one of the different types of training cancer cell samples having a threshold rank for the one of the cell-surface protein genes; and optionally, empirically testing cell-surface expression of the one of the cell-surface protein genes in a test cancer cell sample. The threshold rank can be any user-defined rank as described elsewhere herein.

In some versions, the prediction models of the invention can be used to identify a model cell line for testing a cell-surface targeted therapy in a particular cancer cell type. The methods can comprise: identifying types of the training cancer cell samples having a threshold rank of one of the cell-surface protein genes; identifying a first set of cell lines from the training cell samples having a rank of the one of the cell-surface protein genes comparable to the identified types of the training cancer cell samples; identifying a second set of cell lines from the training cell samples that have a threshold level of protein expression of one of the cell-surface protein genes; identifying the cell lines common to the first set of cell lines and the second set of cell lines to thereby obtain one or more model cell lines; and optionally, testing a therapy targeting the cell-surface protein gene on the one or more model cell lines. The threshold rank can be any user-defined rank as described elsewhere herein. The comparable rank can be defined as any rank being within a user-defined range of the rank of the cell-surface protein genes in the identified types of the training cancer cell samples, or having a high correlation. The testing can comprise contact the one or more model cell lines with an agent of the therapy.

In some versions, the prediction models of the invention encompassing data from single cell samples can be used to predict response and/or toxicity in the treatment of a particular type of cancer. The methods can comprise: identifying a proportion of single cells of a training cancer cell sample from the particular type of cancer having higher than a threshold ranking of one of the cell-surface protein genes; and/or identifying a proportion of single cells of a training non-cancer cell sample having lower than a threshold ranking of the one of the cell-surface protein genes; and optionally, treating the particular type of cancer with a therapeutic targeting the one of the cell-surface protein genes if the proportion of single cells of the training cancer cell sample from the particular type of cancer is higher than a threshold and/or if the proportion of single cells of the training non-cancer cell sample is lower than a threshold. The thresholds can be any user-defined thresholds.

In some versions, the prediction models of the invention can be used to identify two or more cell-surface targets on a particular type of cancer. The methods can comprise: generating a logistic regression model for every pair of the cell-surface protein genes comparing each type of training cancer cell sample and each type of training non-cancer cell sample; identifying pairs in which both cell-surface protein genes in the pair individually contribute to predicting a training cancer cell sample versus a training non-cancer cell sample to obtain predictive pairs; identifying predictive pairs in which the combination of both cell-surface protein genes in the pair discriminate between a training cancer cell sample versus a training non-cancer cell sample.

In some versions, exemplary steps for generating a prediction model as described herein can include collect RNA-seq data from available cancer sequencing datasets; overlapping all datasets to select common genes; ranking all genes from 1 (lowest expression) to N (highest expression) in each sample to make them comparable; and transforming this into a percentile score from 0 to 1.

In some versions, exemplary steps for identify potential clinical cell surface targets for a specific patient can include finding the top N expressed cell surface targets based on RNAseq; employing a minimum threshold as well based on the percentage of housekeeping genes expressed below this target (e.g. 95% of housekeeping genes must be <target); identifying the ranks of the cell surface targets in the prediction model in various training cell samples, and confirming the top N cell surface targets in immunohistochemistry of the tumor sample

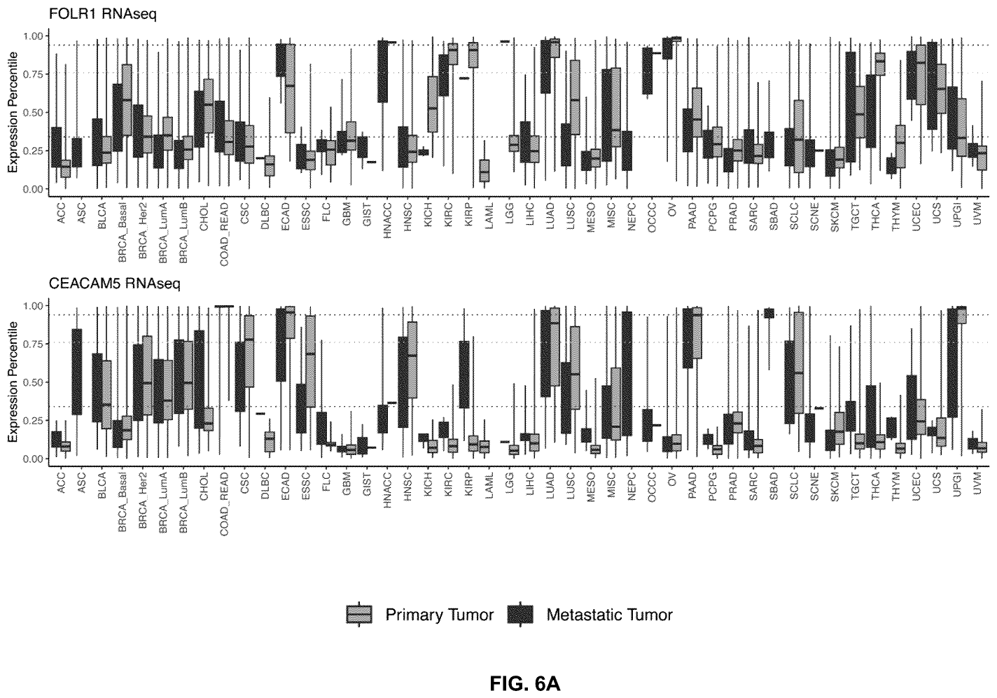

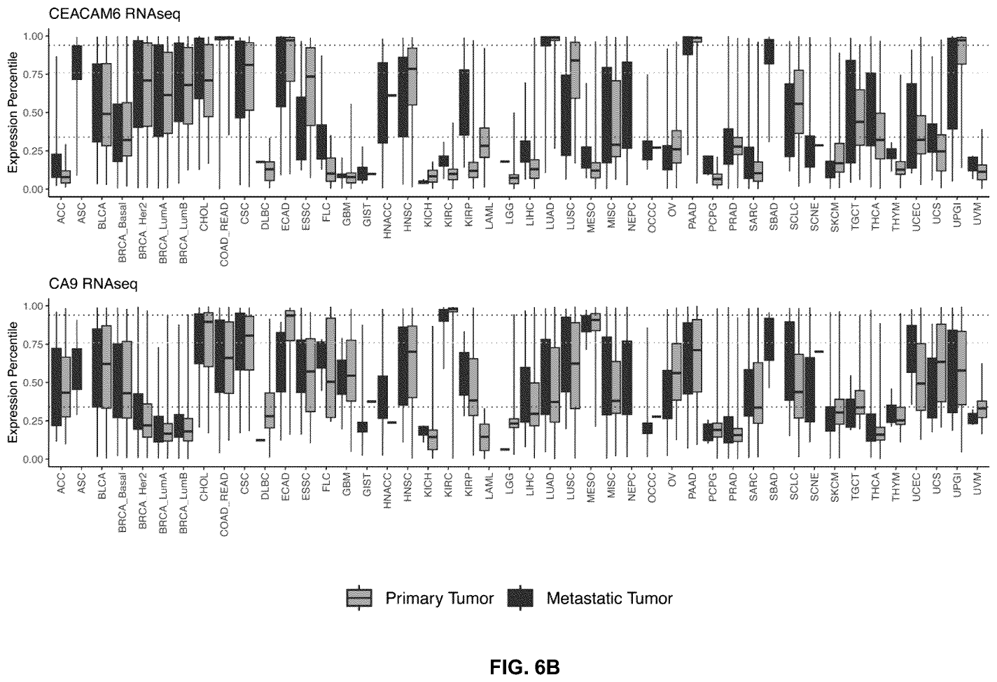

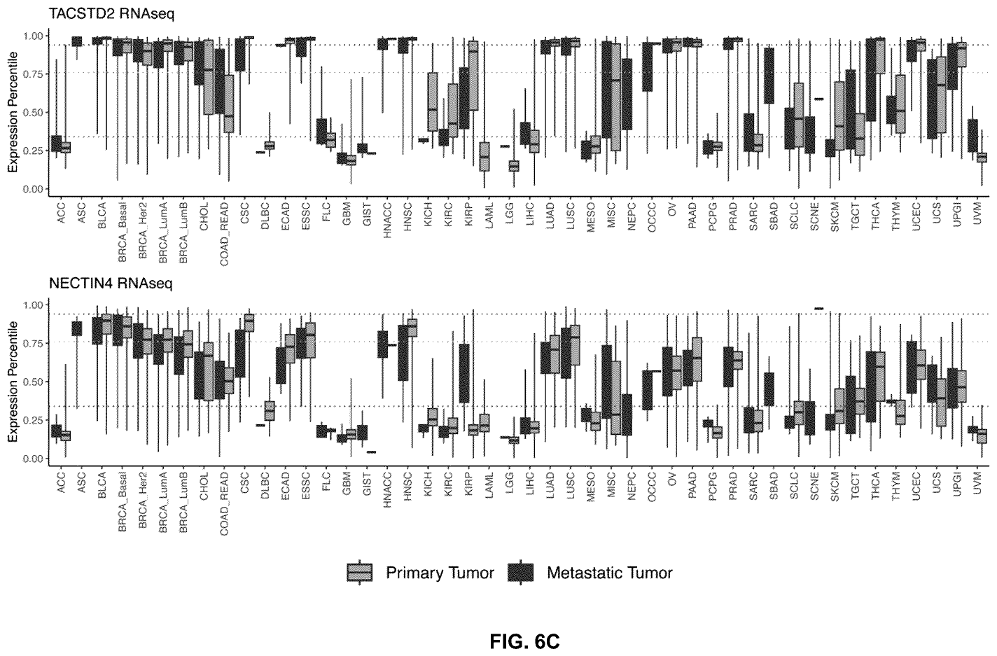

In some versions, exemplary seps to identify potential drug repositioning candidates (FIGS. 6A-6C) include: comparing cell surface target expression of target X across all cancer types, including metastatic and primary tumors; and selecting tumor types where the average expression is high that are not already being investigated.

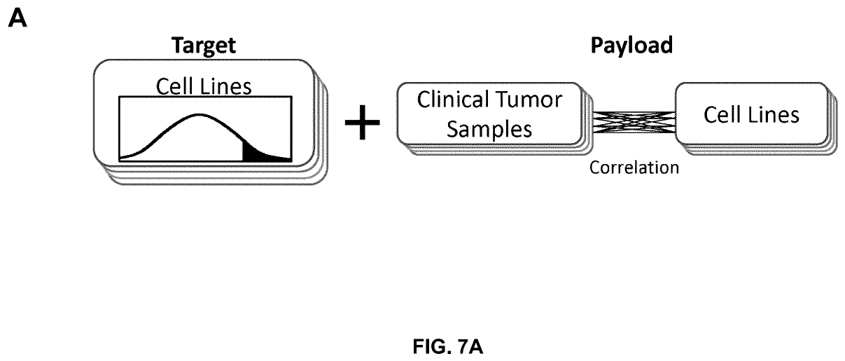

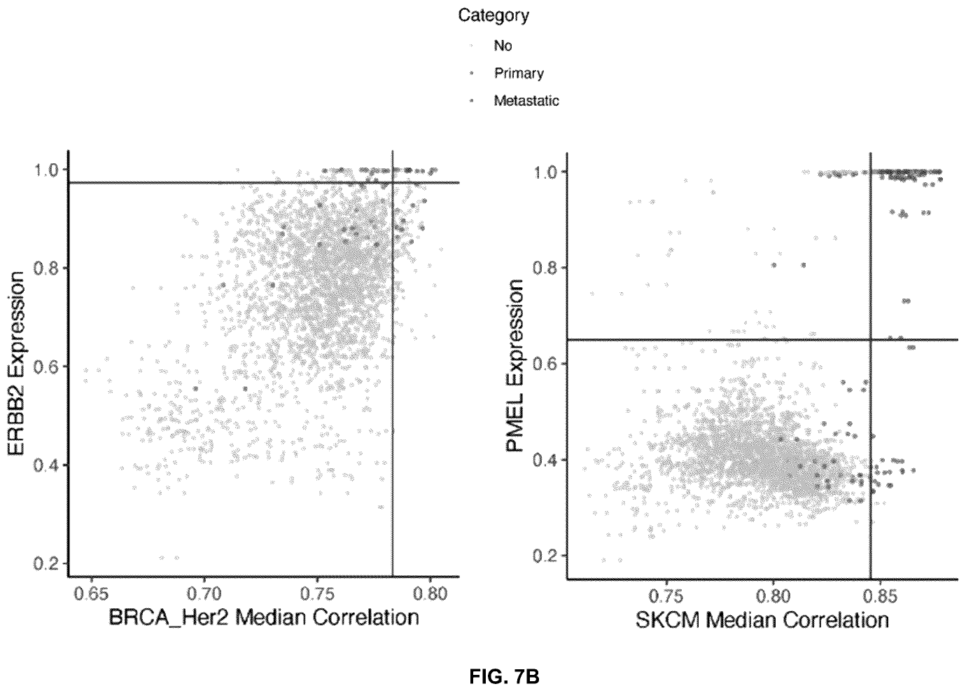

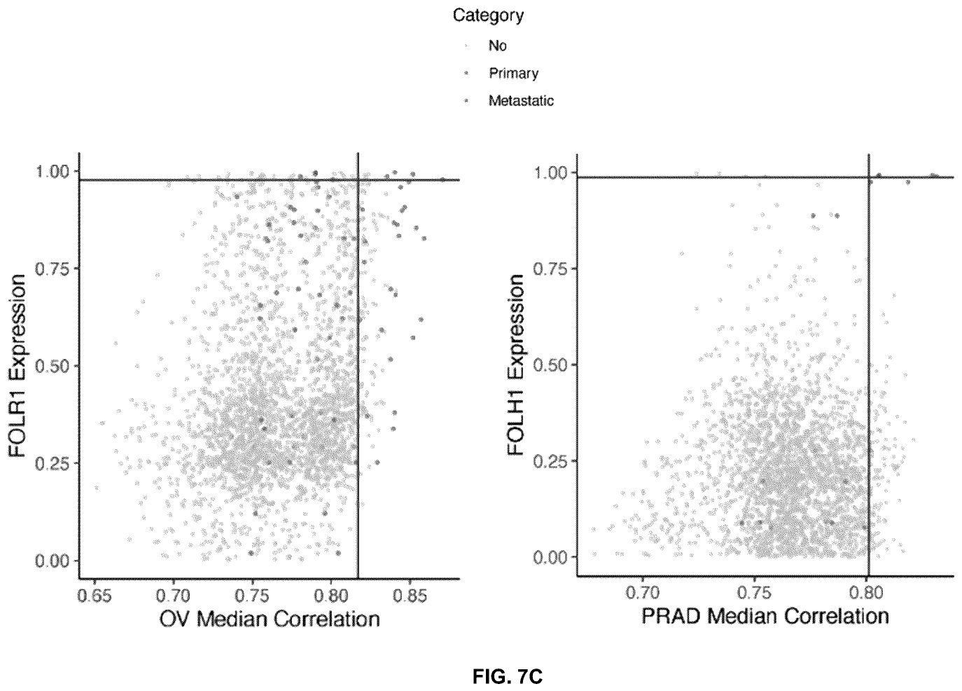

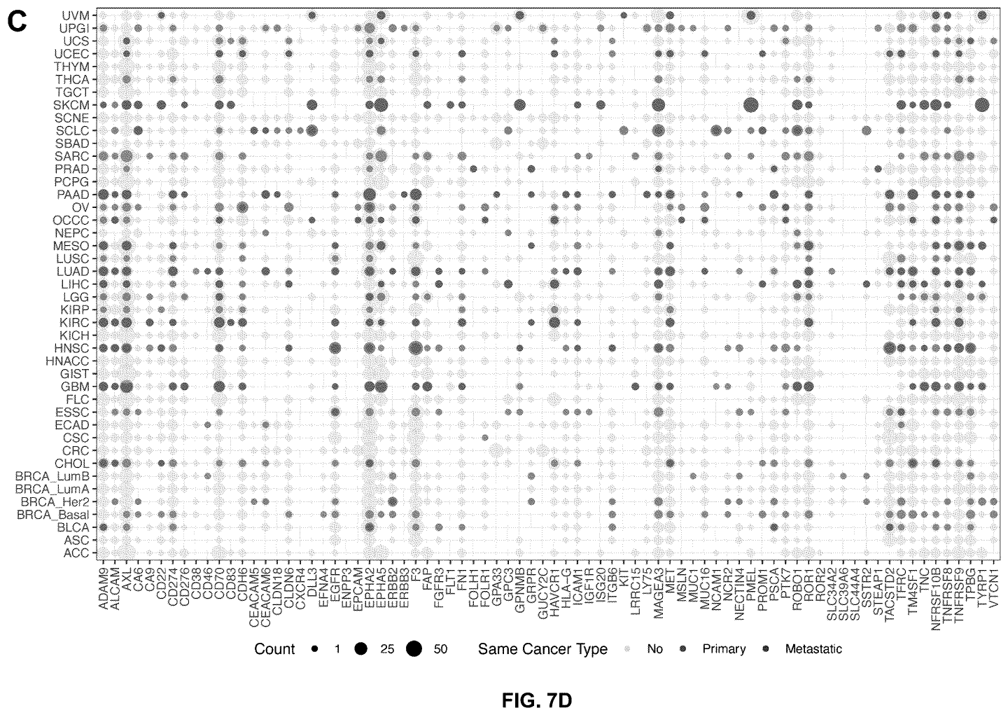

In some versions, exemplary steps to identify a cell line to test a cell surface targeted therapy (FIGS. 7A-7D) include: identifying cell lines with high expression of the target (e.g. top 5% of all genes); identifying cell lines with high average correlation with the tumor type to be studied (e.g. metastatic prostate adenocarcinoma); taking the first two steps, prioritizing those of the same cancer type.

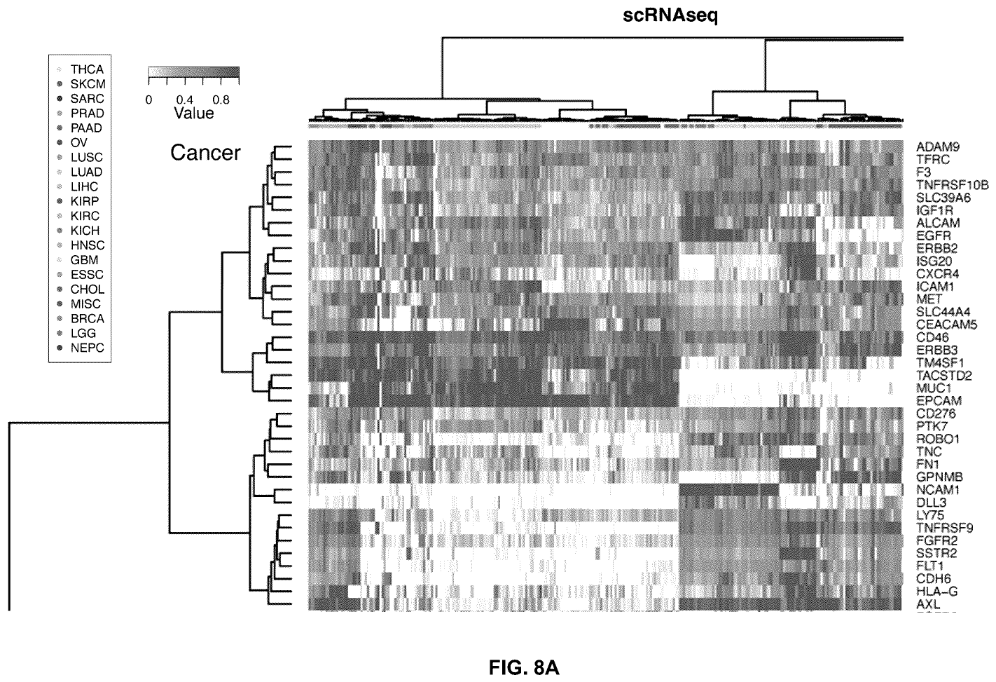

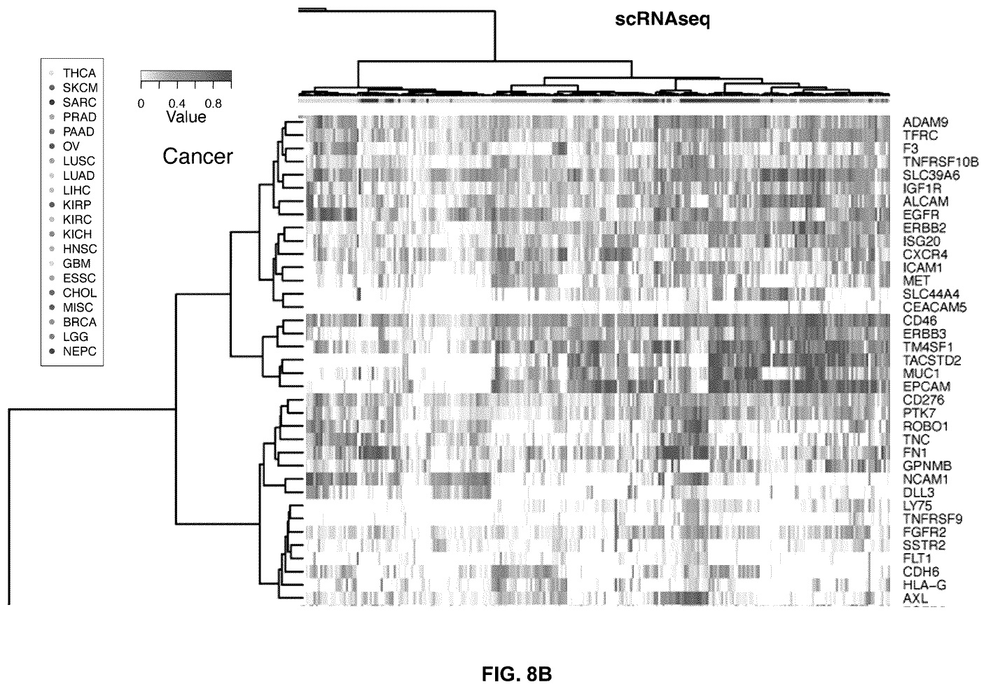

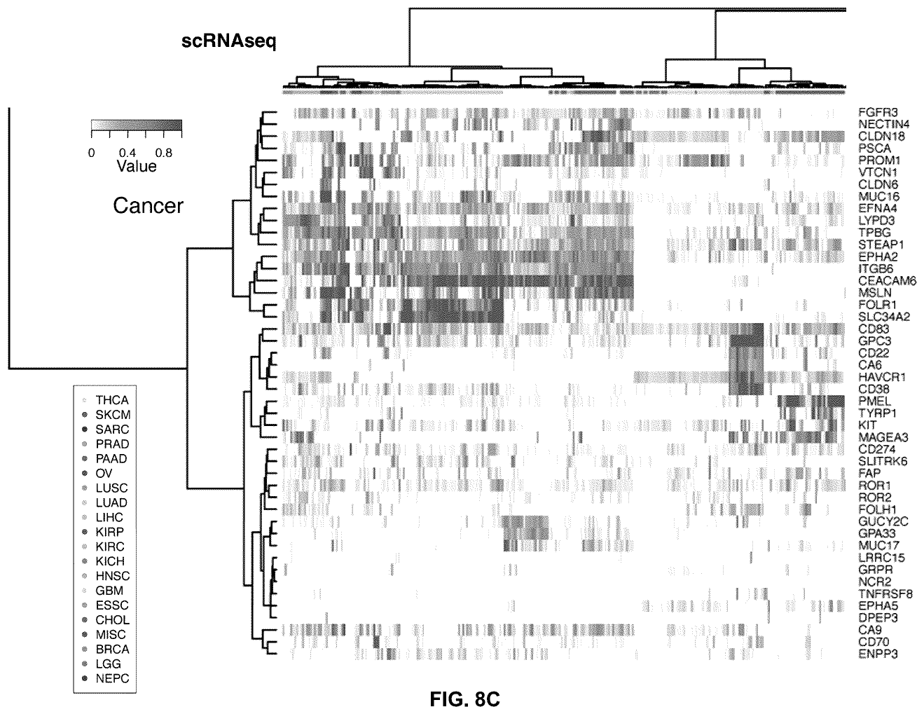

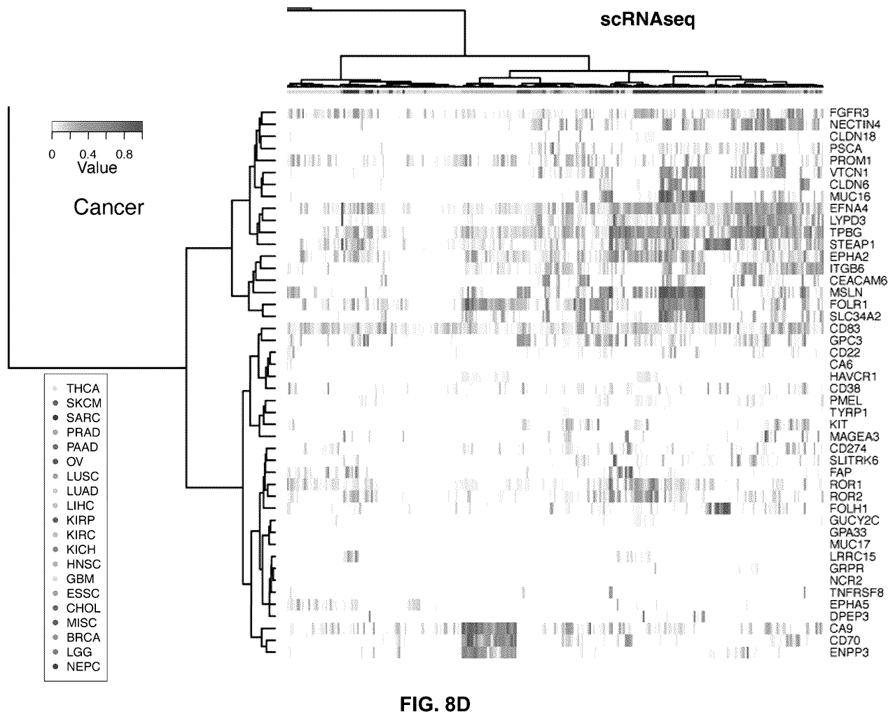

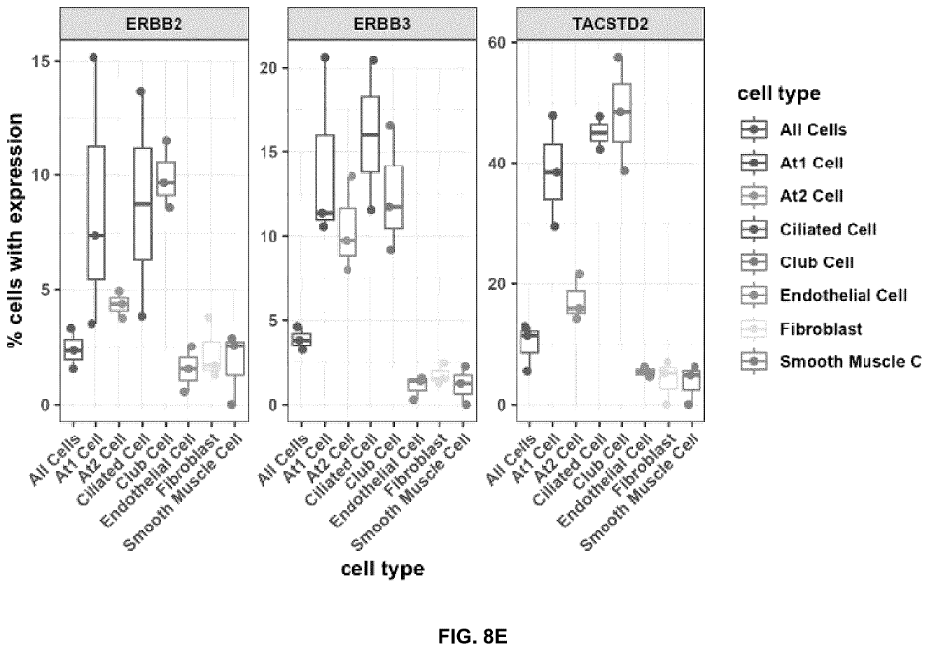

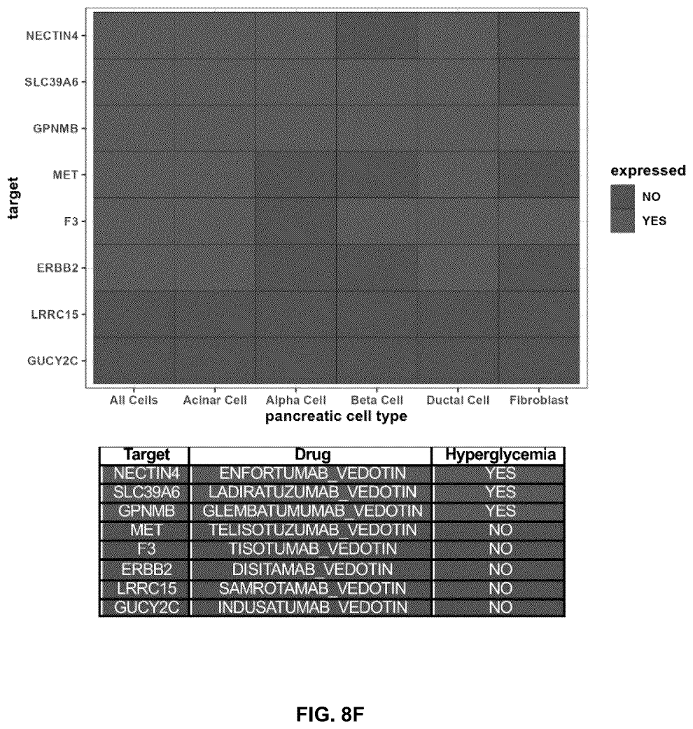

In some versions, exemplary steps to identify rare cancer and normal tissue subtypes with single-cell RNAseq (FIGS. 8A-8D) include: identifying how many tumor cells express (positive vs. negative) a target in a particular cancer type, wherein higher levels of heterogeneity likely predict a worse response to a cell surface targeted therapy; and identifying how many normal cells express (positive vs. negative) a target in a particular normal tissue type, wherein high levels of target expression in a critical normal cell population likely predicts more toxicity from a cell surface targeted therapy.

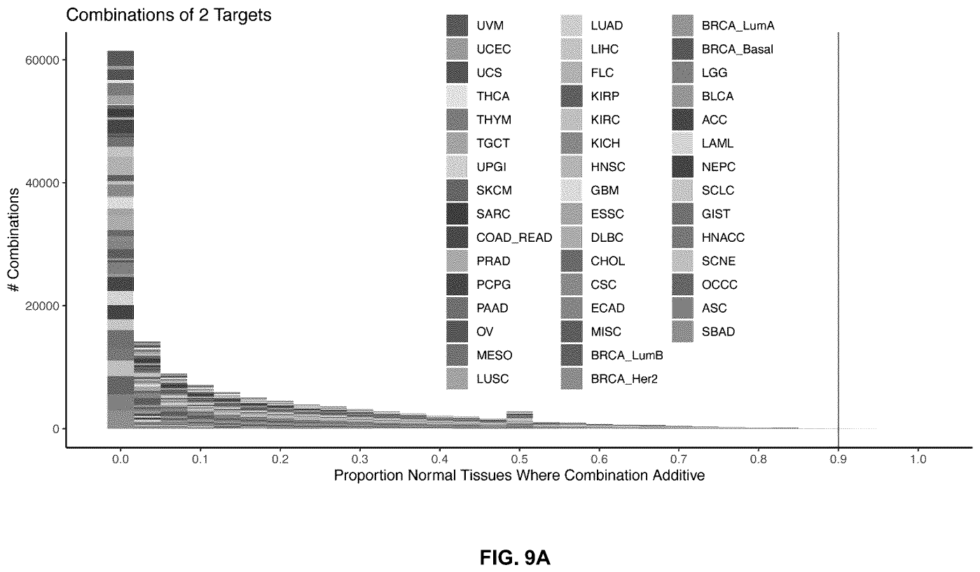

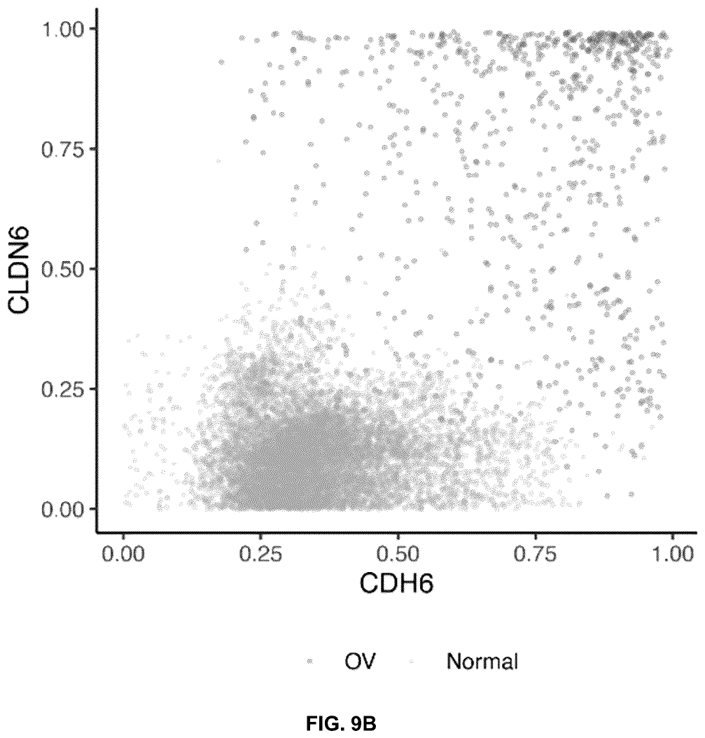

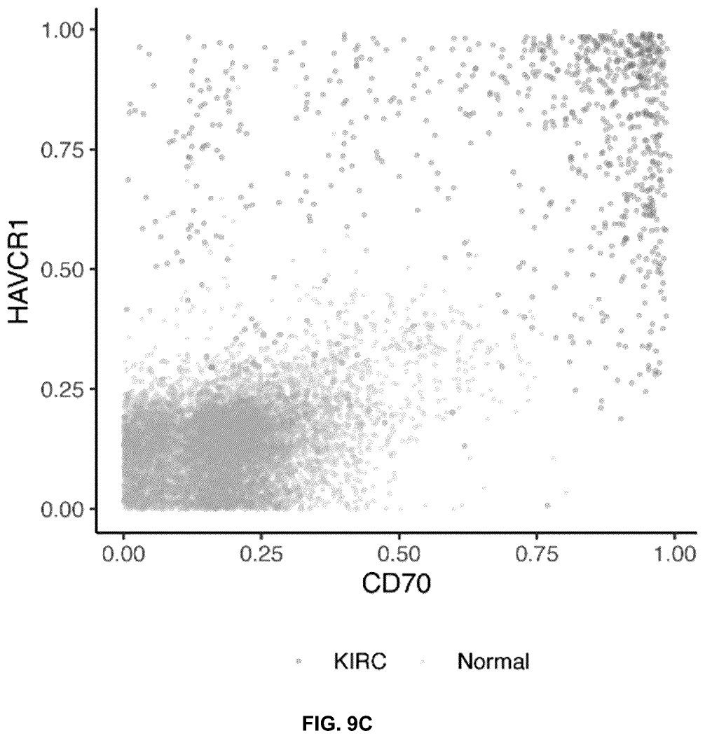

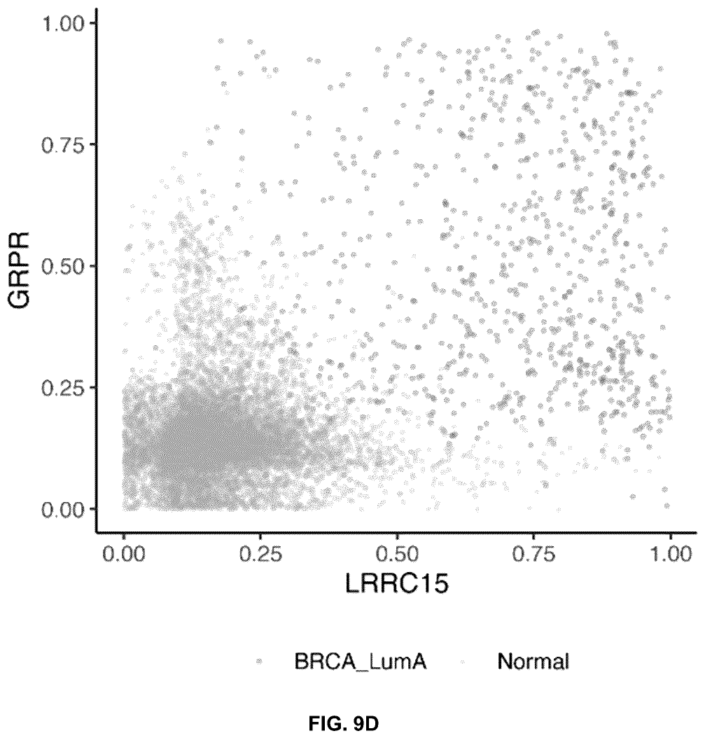

In some versions, exemplary steps to identify pairs of cell surface targets that can provide additive specificity for tumor vs. normal (FIGS. 9A-9D) include: comparing all combinations of two cell surface targets with a logistic regression model for cancer vs. normal; identifying combinations where both targets independently contribute to predicting cancer vs. normal; selecting within these combinations ones where the combination overall discriminates well between cancer vs. normal (e.g. F1 score <0.95).

In some versions, exemplary steps to identify clinical trials targeting specific cell surface targets can include: aggregating all clinical trial metadata from clinicaltrials.gov; identifying the cancer type(s); identifying trials that are open and accruing; identifying the drug used; identifying the cancer drug databases to identify the target(s) of the drug; standardizing the target names (e.g., to their official HGNC gene symbol); and returning the result of open and accruing trials targeting a specific target.

Another aspect of the invention is directed to a prediction model generated as described herein. The prediction model can be stored and operated from a memory and/or processor.

The use of “we,” “our,” and other plural referents used herein are to refer to the present inventor and in no way implies multiple inventors.

The elements and method steps described herein can be used in any combination whether explicitly described or not.

All combinations of method steps as used herein can be performed in any order, unless otherwise specified or clearly implied to the contrary by the context in which the referenced combination is made.

As used herein, the singular forms “a,” “an,” and “the” include plural referents unless the content clearly dictates otherwise.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 5 to 6, from 1 to 9, from 3.6 to 4.6, from 3.5 to 9.9, and so forth.

All patents, patent publications, and peer-reviewed publications (i.e., “references”) cited herein are expressly incorporated by reference to the same extent as if each individual reference were specifically and individually indicated as being incorporated by reference. In case of conflict between the present disclosure and the incorporated references, the present disclosure controls.

It is understood that the invention is not confined to the particular construction and arrangement of parts herein illustrated and described, but embraces such modified forms thereof as come within the scope of the claims.

### EXAMPLES

### Example 1. Clinical Cell Surface Targets in Metastatic and Primary Solid Cancers

### Summary

Cell-surface targeted (CST) therapies represent an emerging treatment class in solid malignancies. However, high-throughput investigations of CST expression across cancer types have been reliant on datasets of mostly primary tumors, despite therapeutic use most commonly in metastatic disease. We identified a total of 818 clinical trials of CST therapies with 78 CSTs. We assembled a dataset spanning RNA-seq and microarrays in 7927 benign samples, 16866 primary tumor samples, and 6124 metastatic tumor samples. We also utilized single-cell RNA-seq data from 36 benign tissues and 558 primary and metastatic tumor samples, and matched RNA vs. protein expression in 29 benign tissue samples, 1075 tumor samples, and 942 cell lines. High RNA expression accurately predicted high protein expression across CSTs in benign tissues, tumor samples, and cell lines. We compared metastatic vs. primary tumor expression, identified potential opportunities for repositioning, and matched cell lines to tumor types based on CST and global RNA expression. We evaluated single-cell heterogeneity across tumors, and identified rare normal cell sub-populations that may contribute to toxicity. Finally, we identified combinations of CSTs, for which bi-specific approaches could improve tumor specificity. This study helps better define the landscape of CST expression in metastatic and primary cancers.

### Introduction

To date, the identification of cell surface markers for new therapeutic targets beyond well-established tumor cell surface proteins such as HER2 and PSMA has utilized data from large sequencing datasets such as The Cancer Genome Atlas (TCGA). These data are primarily obtained from non-metastatic tumor tissue samples, due to the challenges of profiling large metastatic tissue datasets. However, tumor expression profiles have been shown to differ in metastatic disease (1), suggesting a gap in knowledge of optimal cell surface targets in the metastatic setting. To address this, we curated a list of cell surface targets with agents currently under study in clinical trials. We established in paired protein-RNA datasets that high gene expression was strongly associated with high protein expression of these targets. We then evaluated these targets in a large integrated RNA expression dataset of metastatic tumors paired with primary tumors to understand patterns of cell surface target expression between and within tumor types. We further integrated normal tissue RNA sequencing as well as normal and tumor tissue single-cell RNA-seq (scRNA-seq) datasets into our analysis to understand the contribution of normal tissue expression and single cell heterogeneity to potential efficacy and toxicity profiles across these cell surface targets.

### Results

### Overview of Cell Surface Targets in Clinical Trials/Practice

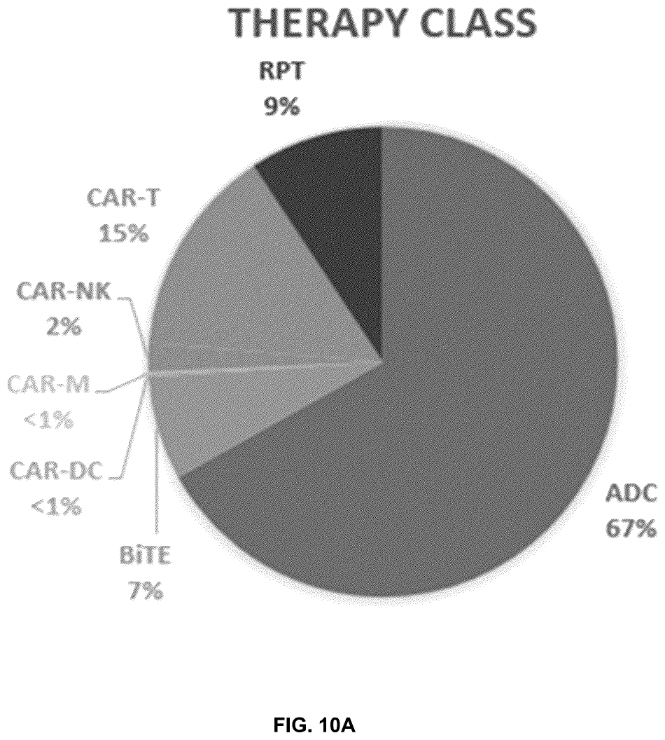

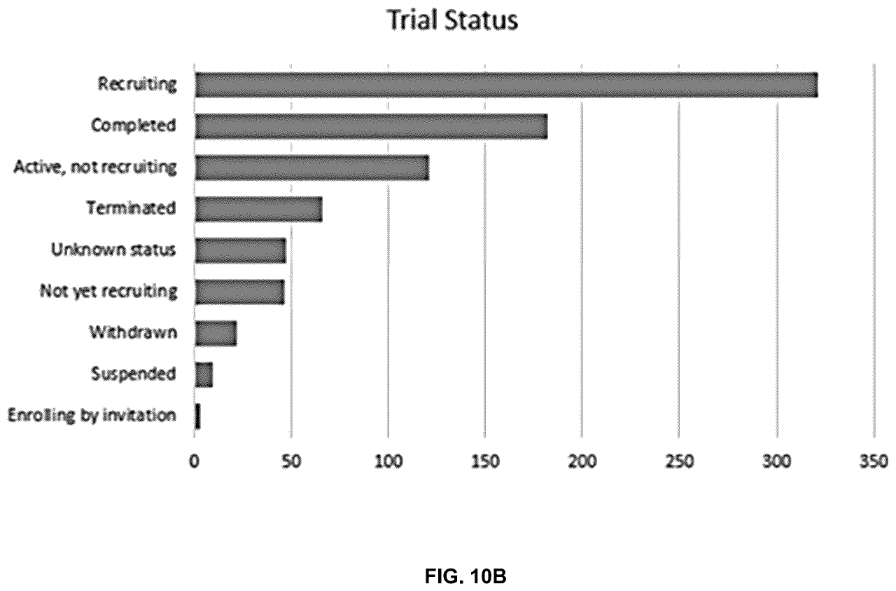

We first performed a comprehensive search of ClinicalTrials.gov to identify all interventional clinical trials (as of Oct. 31, 2023) of surface-targeted cancer therapies for adult solid tumors. We identified a total of 818 trials. We manually curated the cell surface protein targets, inclusion criteria, and phase of each trial based on information from trial details and related publications. A total of 78 cell-surface protein targets were identified including 9 targets with FDA-approved therapies for solid tumors. Of these trials, the majority (65%) assessed ADCs (FIG. 10A), and 82% were early phase (I-II) trials. Given that cell surface targeted therapies represent an emerging modality, most of the clinical trials were currently active at the time of review (FIG. 10B).

### High-Throughput Datasets

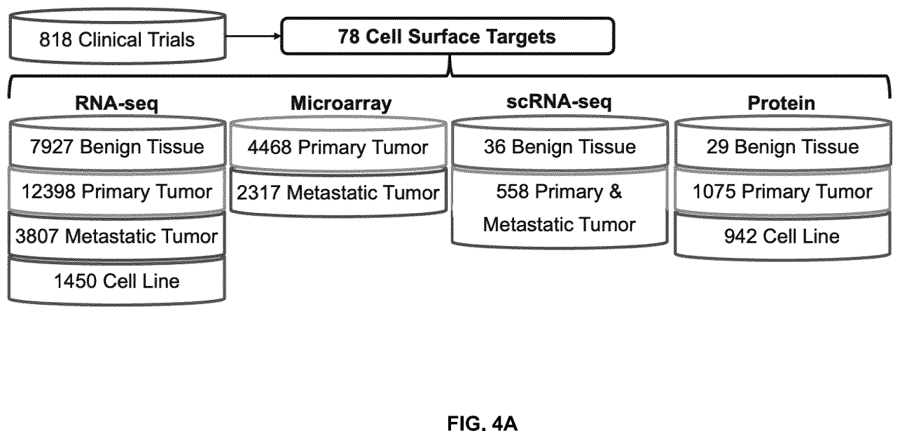

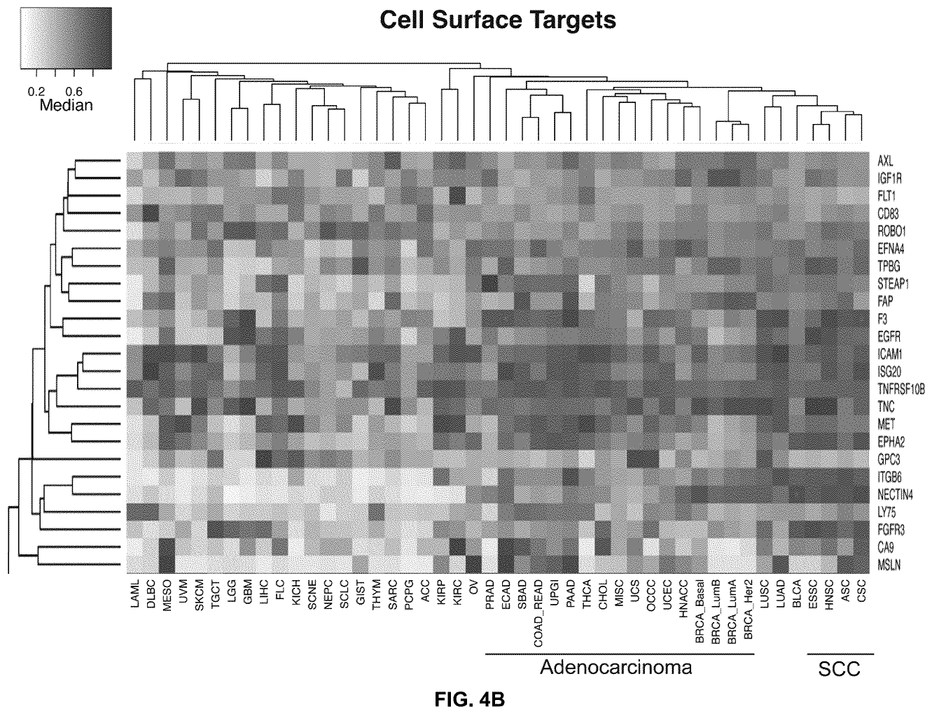

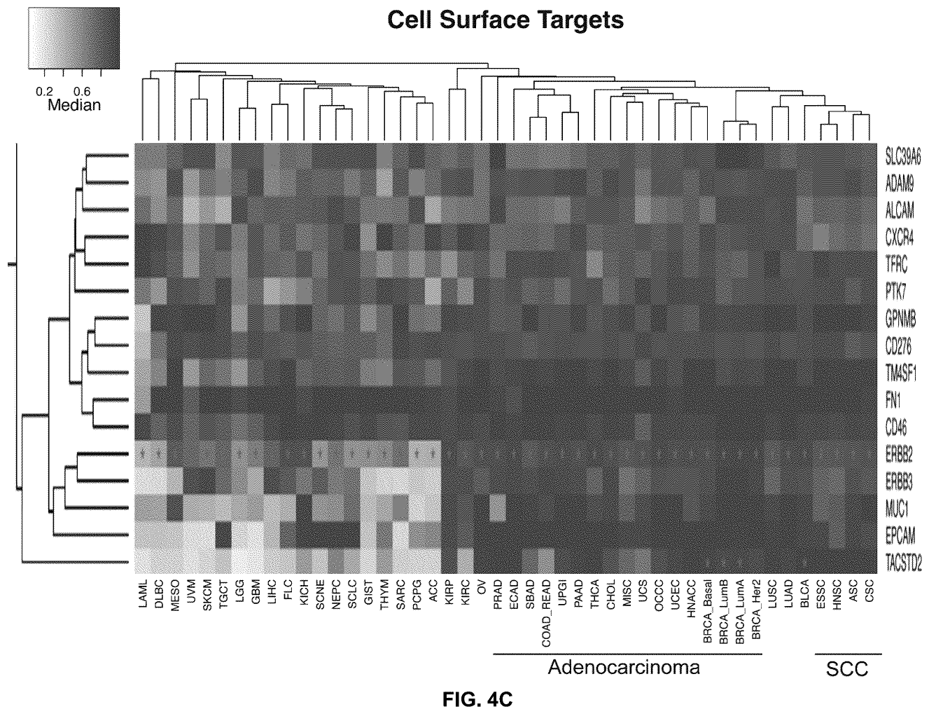

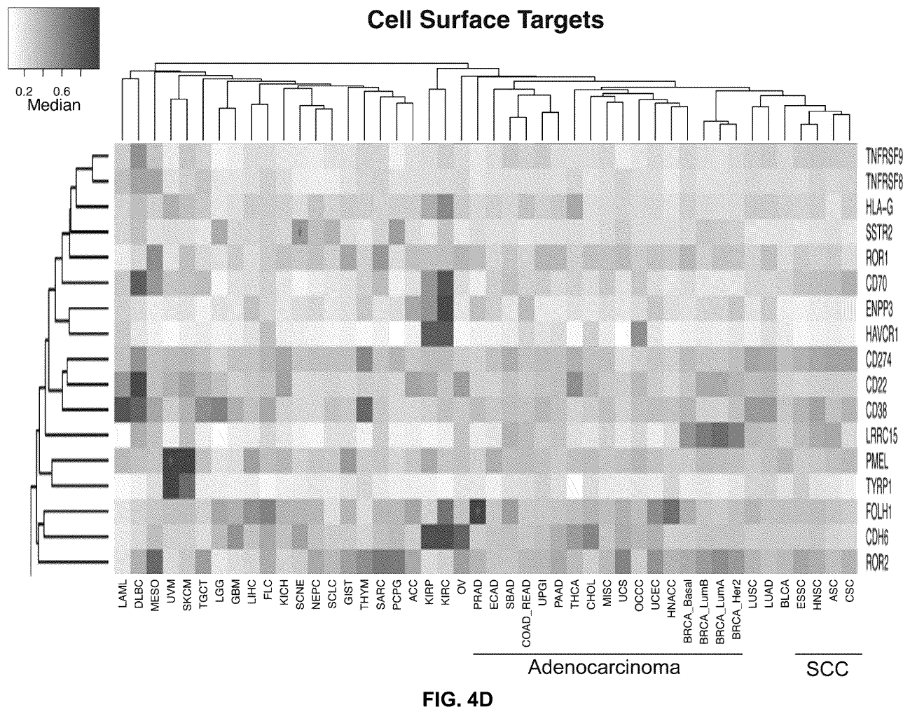

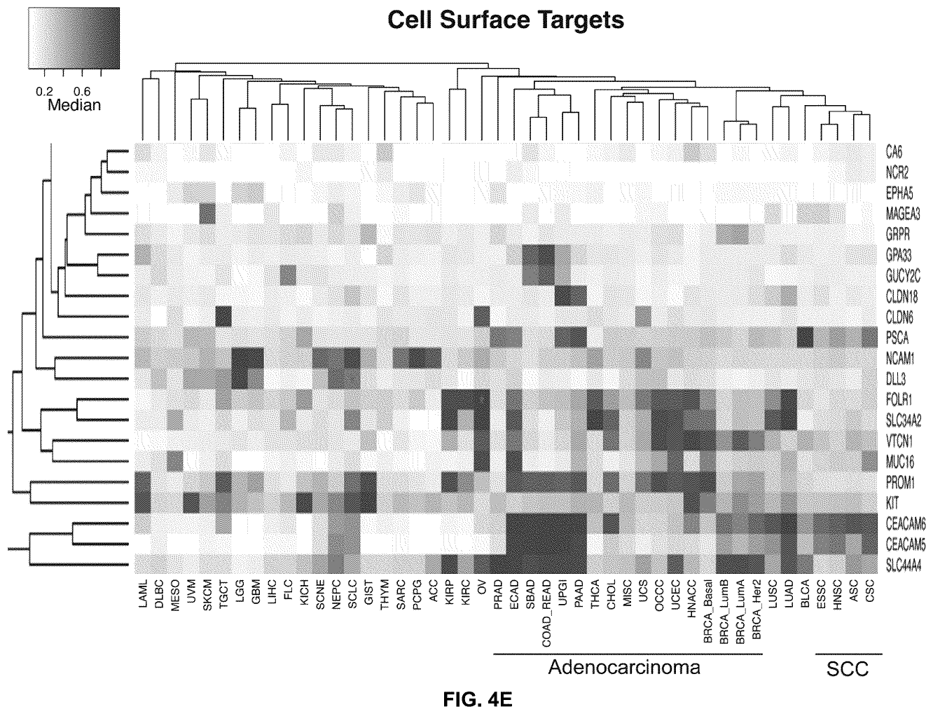

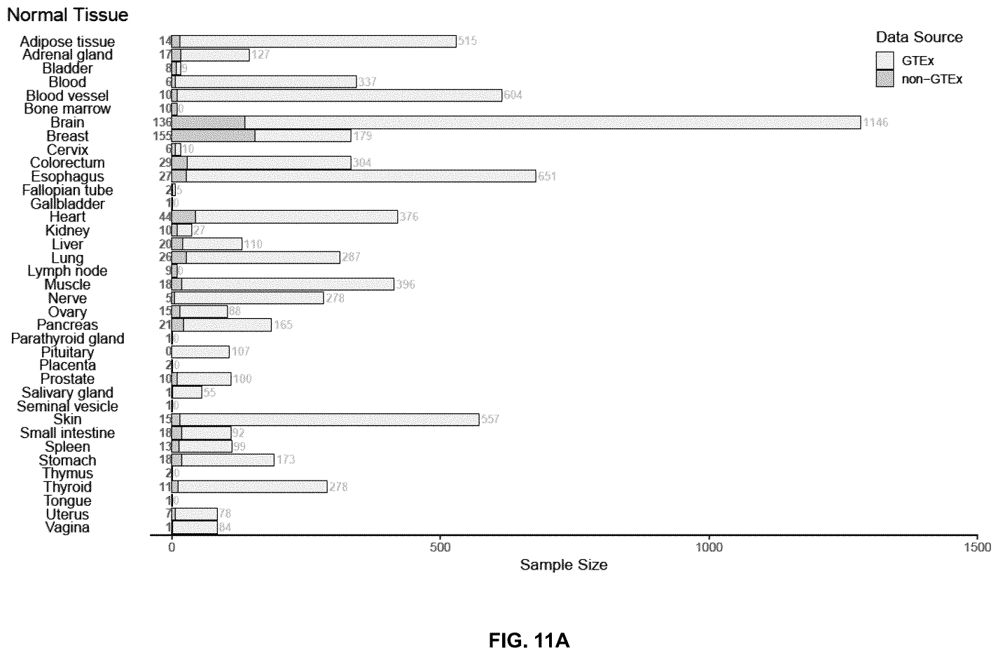

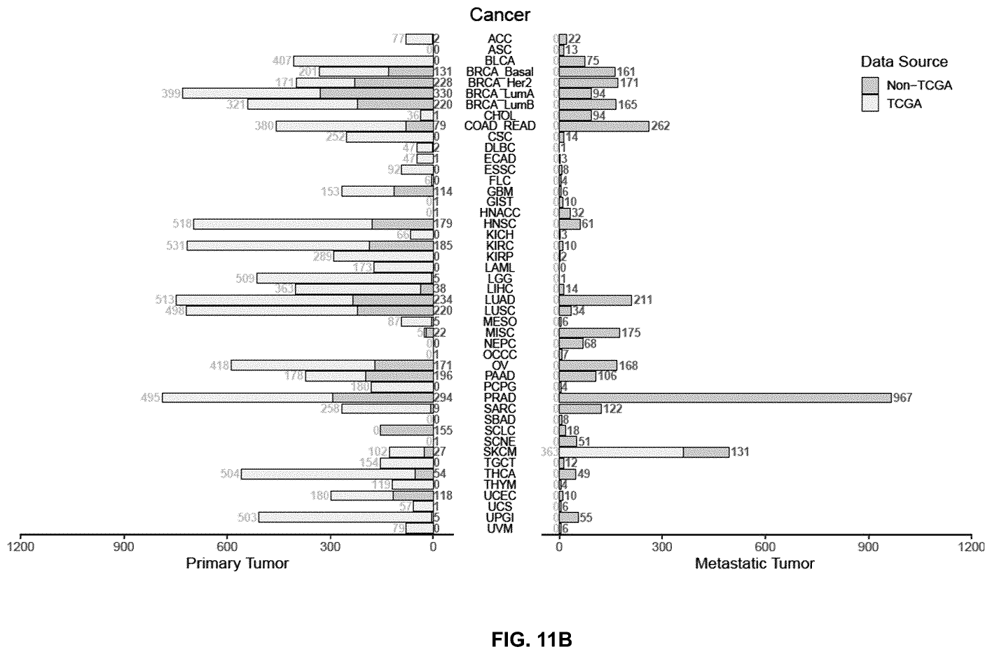

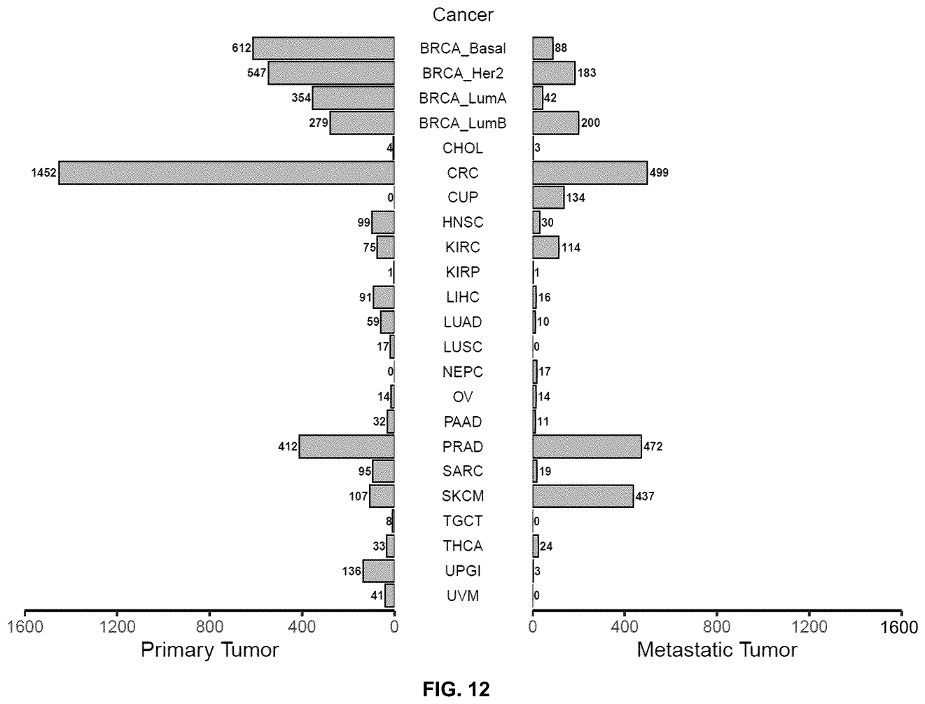

To best understand how cell surface target expression varies across cancer types, benign tissues, and cell lines, we assembled a large dataset spanning RNA-seq data in 7927 benign samples, 12398 primary tumor samples (mostly from TCGA), and 1450 cell lines (FIG. 4A). Additionally, we also included 3807 metastatic tumor samples (FIGS. 11A and 11B). In addition to RNA-seq, we also included RNA expression from 4468 primary tumor samples and 2317 metastatic tumor samples profiled with gene expression microarrays (FIG. 12). To facilitate comparisons between disparate datasets, we normalized each gene to the percentile expression within each sample. We also utilized single-cell RNA-seq data from 36 benign tissues and 558 primary and metastatic tumor samples. Finally, we also compared matched RNA vs. protein expression in 29 benign tissue samples, 1075 tumor samples, and 942 cell lines. In aggregate, these data allow us to comprehensively explore cell surface target expression across solid tumors.

### High Cell Surface Target RNA Expression Accurately Predicts High Protein Expression

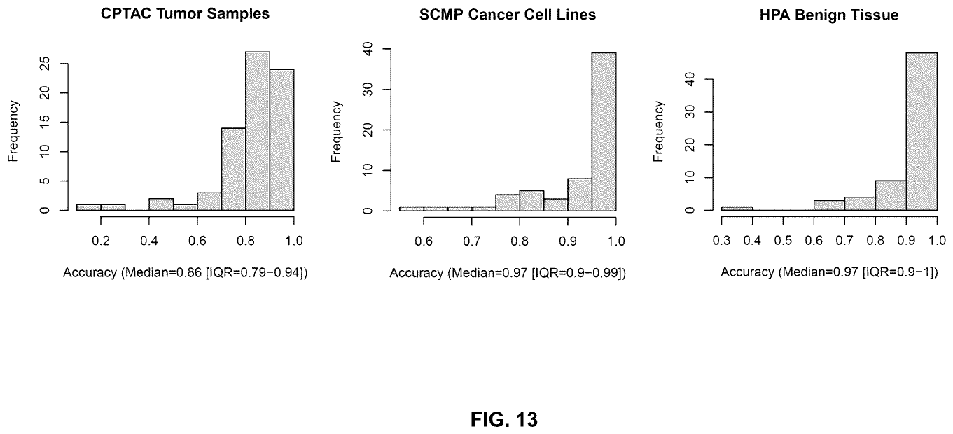

We first examined cell surface RNA vs. protein expression in three datasets of cancer tissue samples, cell lines, and benign tissues with matched RNA-seq and mass spectrometry. For cell surface expression, our focus was on how accurately high RNA expression associated with high protein expression rather than the exact degree of linear correlation. We therefore defined high expression of protein or RNA expression as being higher than 90% of housekeeping gene protein or RNA expression respectively in each sample. In clinical cancer samples from the Clinical Proteomic Tumor Analysis Consortium (CPTAC), the median accuracy of high RNA predicting high protein expression (and therefore low RNA predicting low protein expression) across cell surface targets was 86% [IQR 79%-94%]. Similarly, in cell lines from the Sanger Cell Model Passports (SCMP) (2), the median accuracy of high RNA predicting high protein expression across cell surface targets was 97% [IQR 90%-99%]. Finally, in benign tissue samples from the Human Protein Atlas (HPA) (3), the median accuracy of high RNA predicting high protein expression across cell surface targets was 97% [IQR 90%-100%]. These data demonstrate that high RNA expression can be used to accurately infer high protein expression in many tumors (FIG. 13). The inaccuracy rate (low RNA expression predicting high protein expression, or high RNA expression predicting low protein expression) can be inferred from 100% minus these rates.

### Cell Surface Target Expression Across Cancers

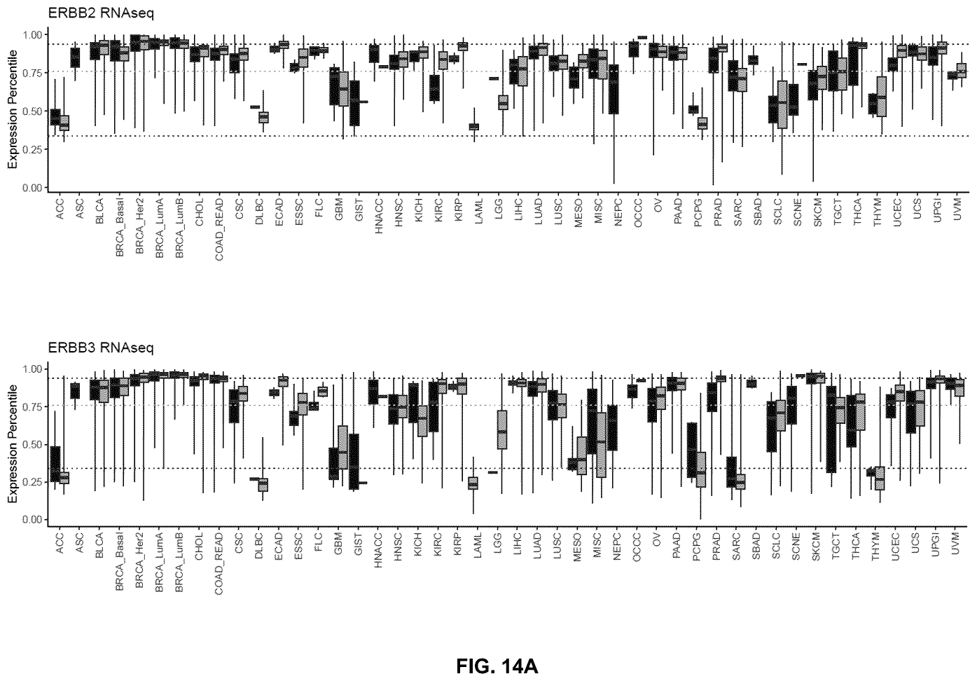

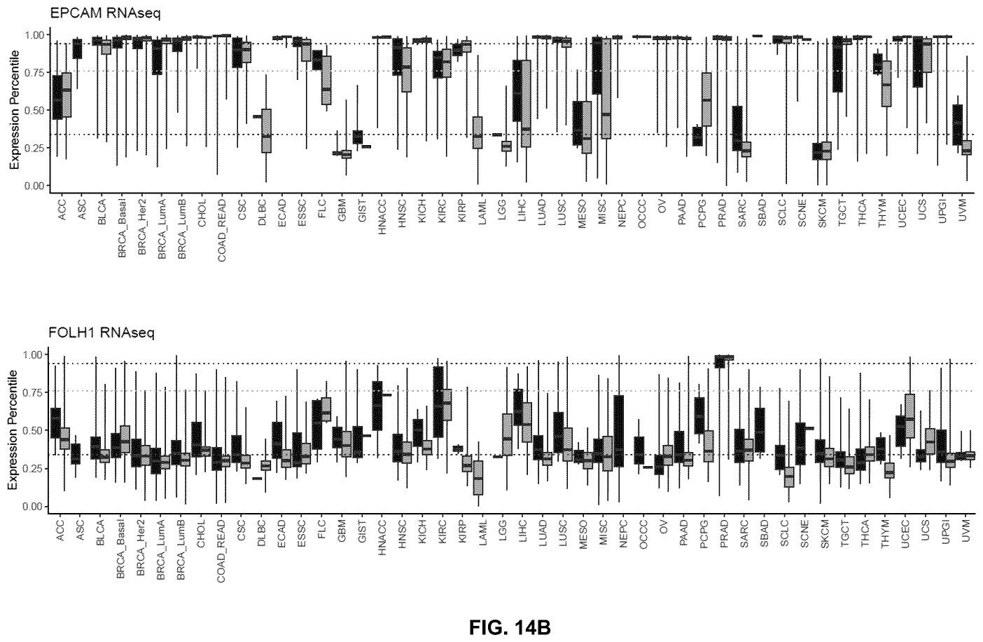

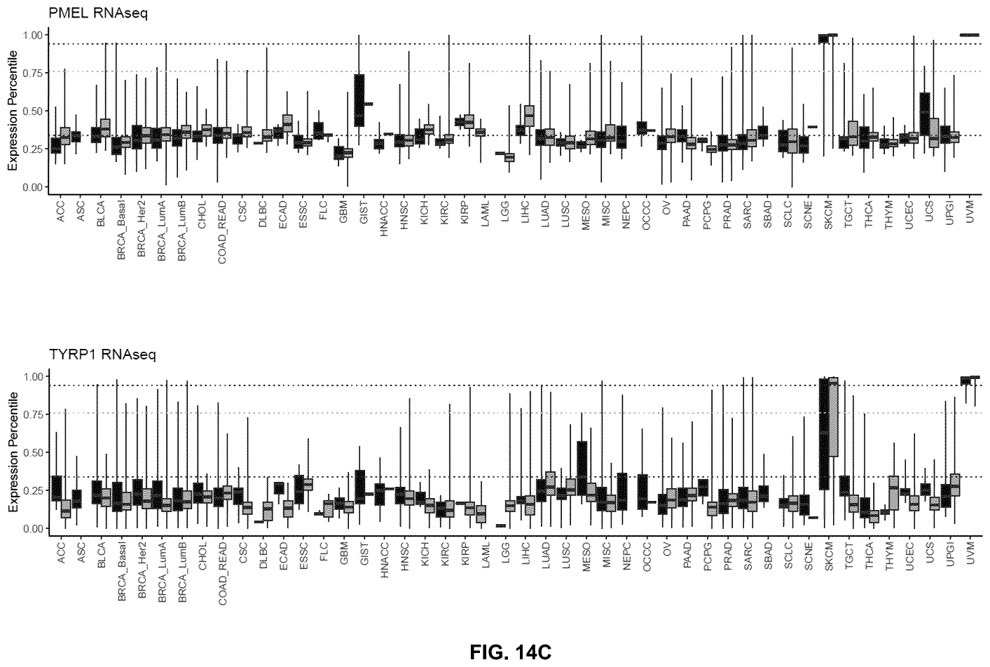

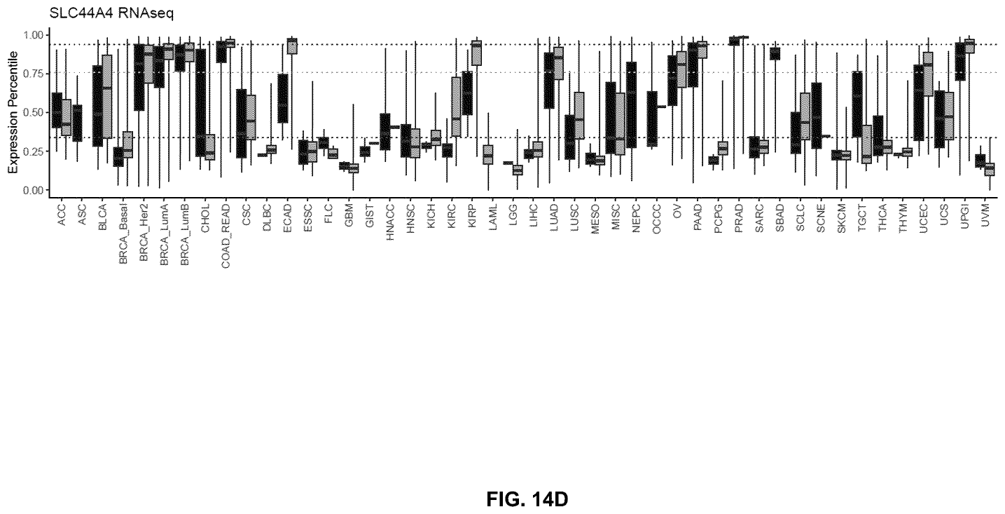

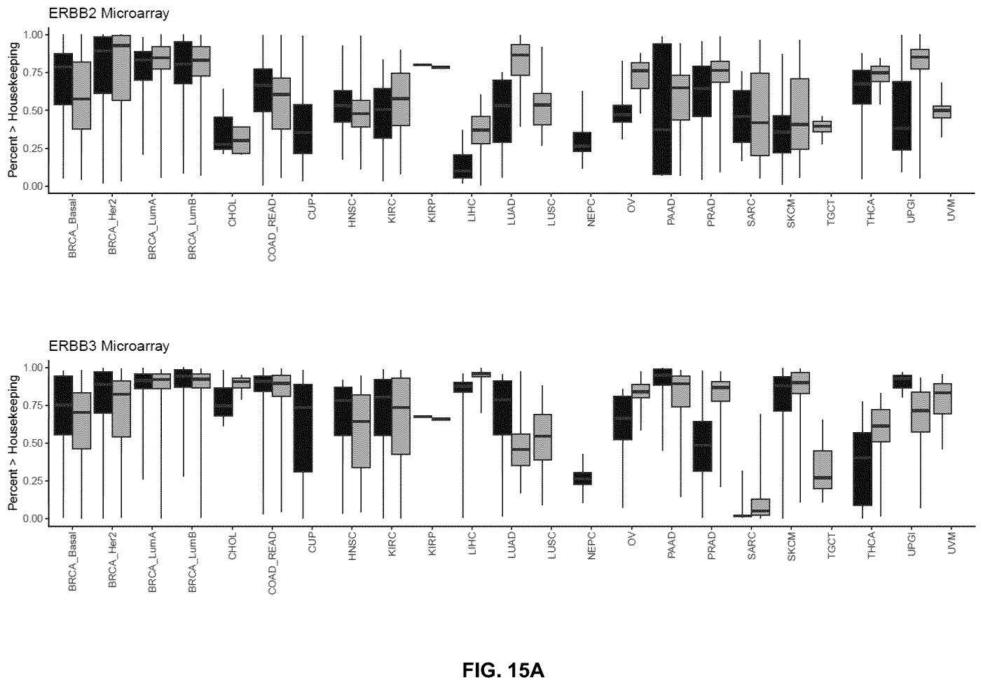

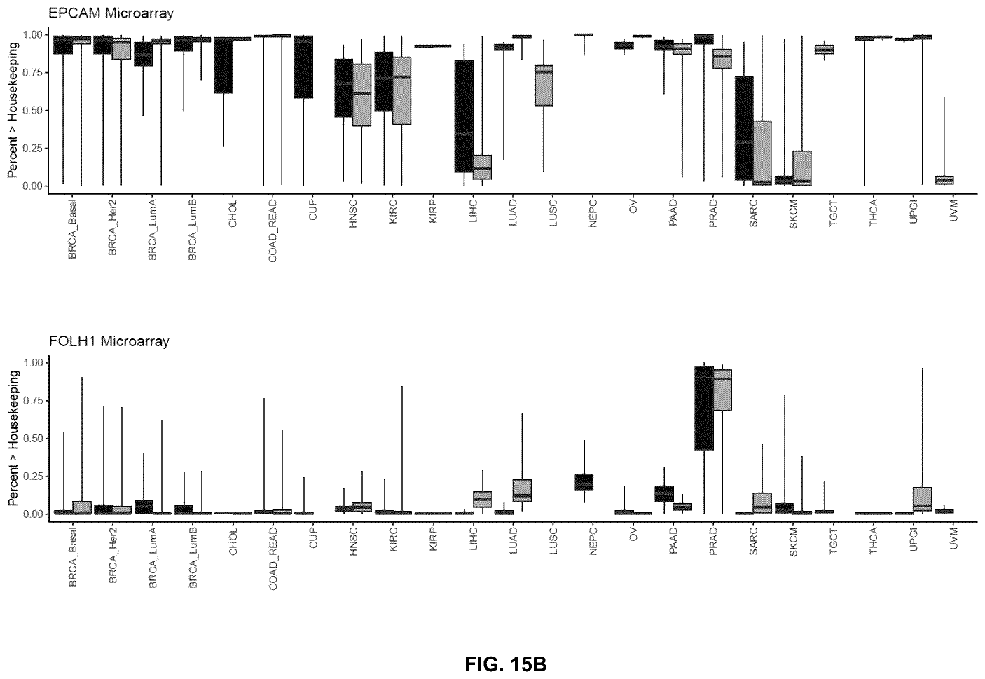

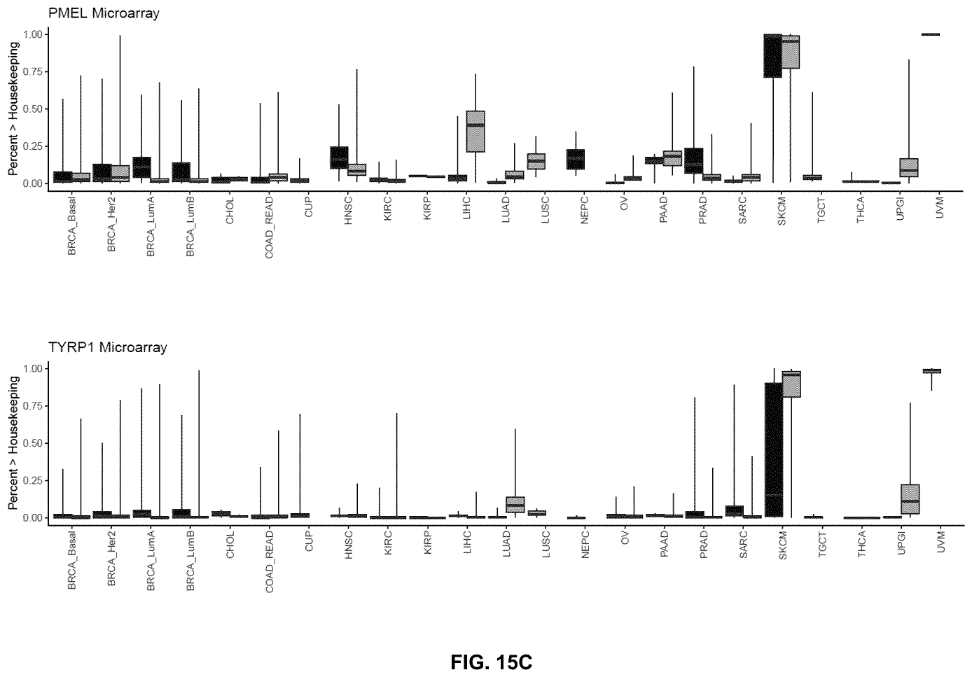

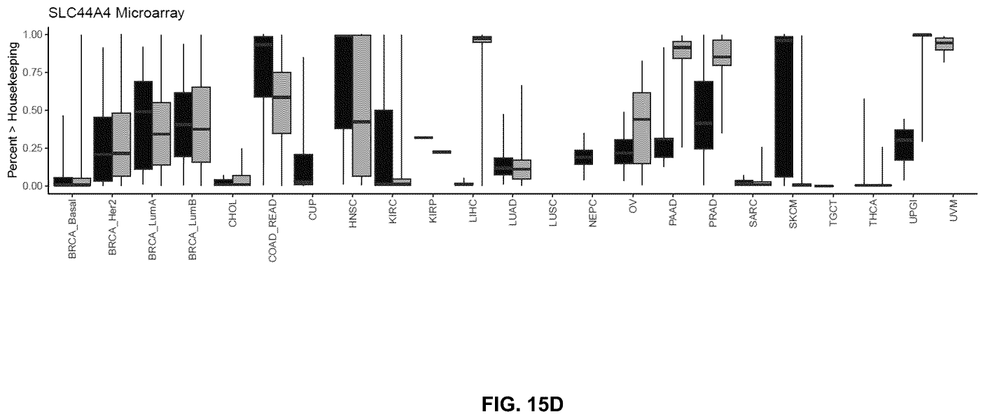

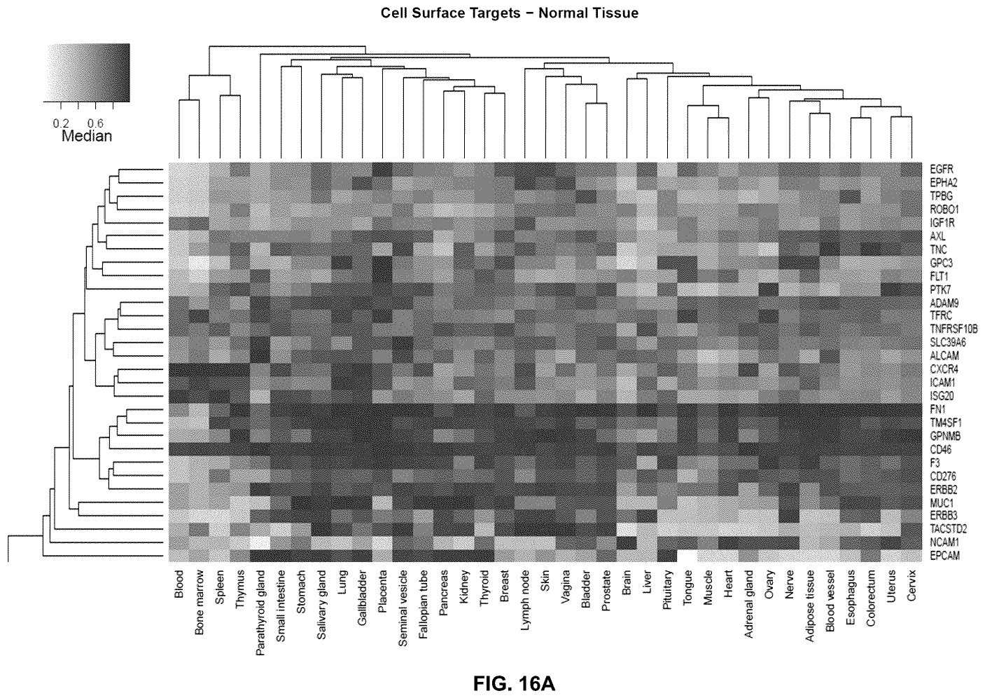

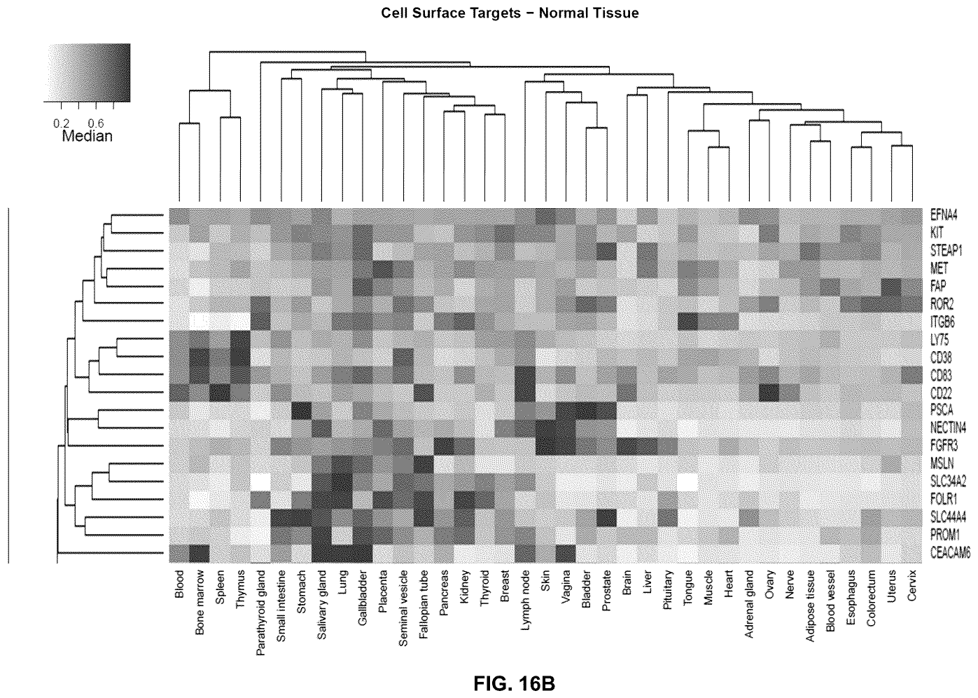

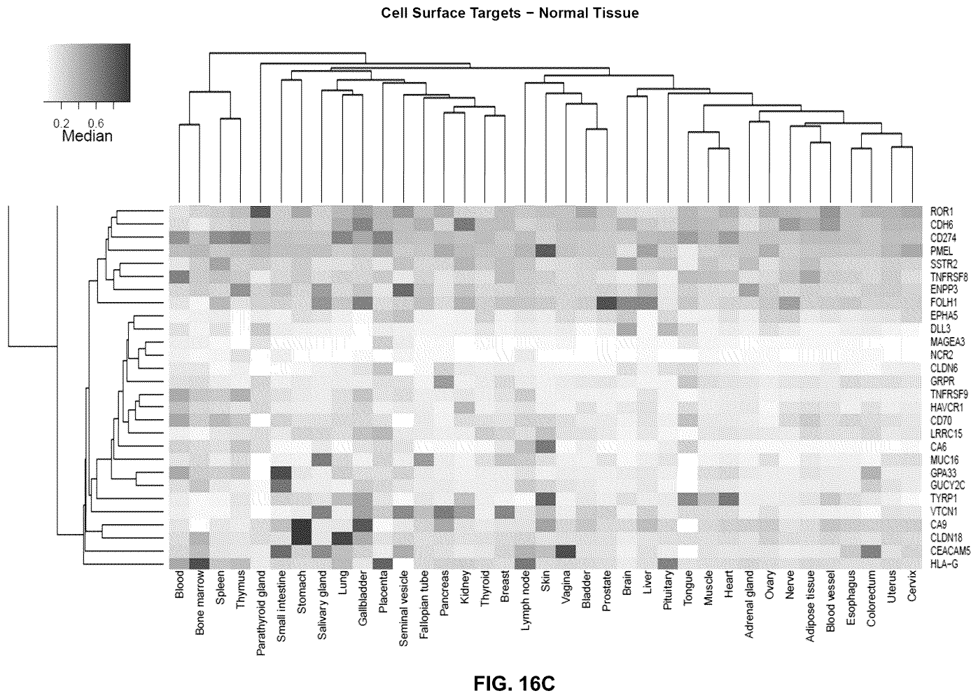

We first evaluated expression of the cell surface targets across the benign and tumor tissues in our integrated datasets. Broadly speaking, hierarchical clustering of tumor types by cell surface target expression clustered by histology of adenocarcinoma versus non-adenocarcinoma (FIGS. 4B-4E). About half of the cell surface targets were more ubiquitous and highly expressed, including some across all tumor types such as CD276 (B7-H3), FN1, and GPNMB. Across adenocarcinoma histology subtypes ERBB2 (HER2), ERBB3 (HER3), EPCAM, TACSTD2 (TROP2), and NECTIN4 were all found to be highly expressed. Other cell surface targets had more restricted expression by tumor type including FOLR1 which was detected in gynecologic malignancies, FOLH1 (PSMA) in prostate cancer, PMEL (gp100) and TYRP1 in cutaneous and uveal melanoma, and SCL44A4, CEACAM5 and CEACAM6 in gastrointestinal (GI) malignancies (FIGS. 14A-14D). These results were confirmed in our microarray data as well (FIGS. 15A-15D). Notably, these findings are concordant with the tumor type/cell surface target pairings for which therapies directed against these targets are currently in clinical trials. In the set of benign tissues, as expected some targets were ubiquitously highly expressed, while others show more restricted or universally low expression (FIGS. 16A-16C). Interestingly, FDA-approved agents exist for targets in each of these categories, including ERBB2 (ubiquitous expression), TACSTD2, FOLR1, NECTIN4 (restricted expression), and FOLH1 (low expression).

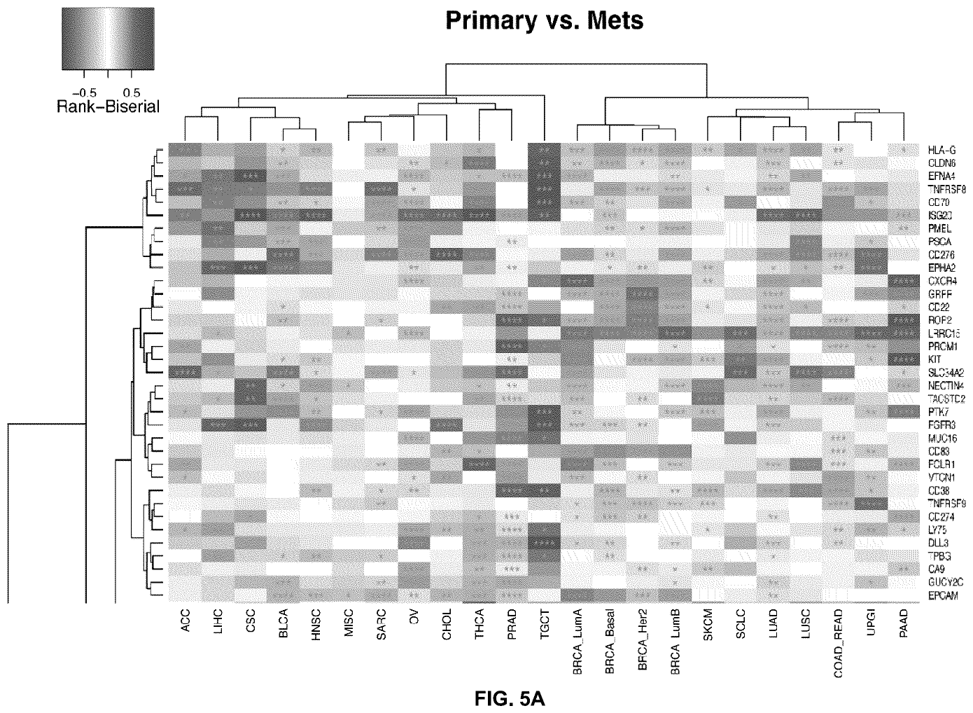

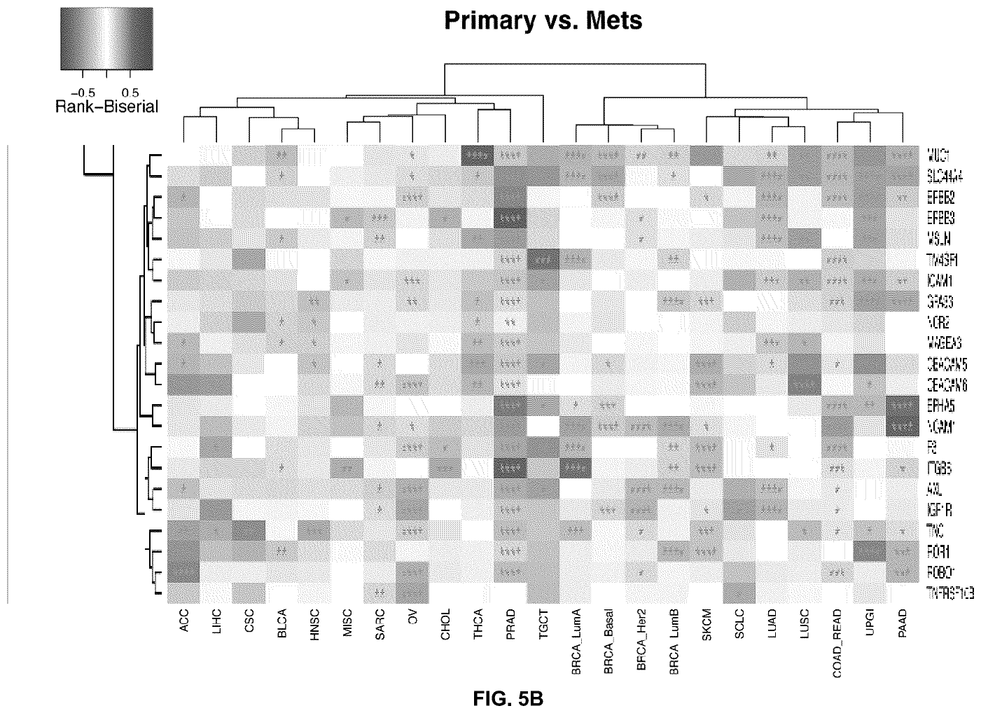

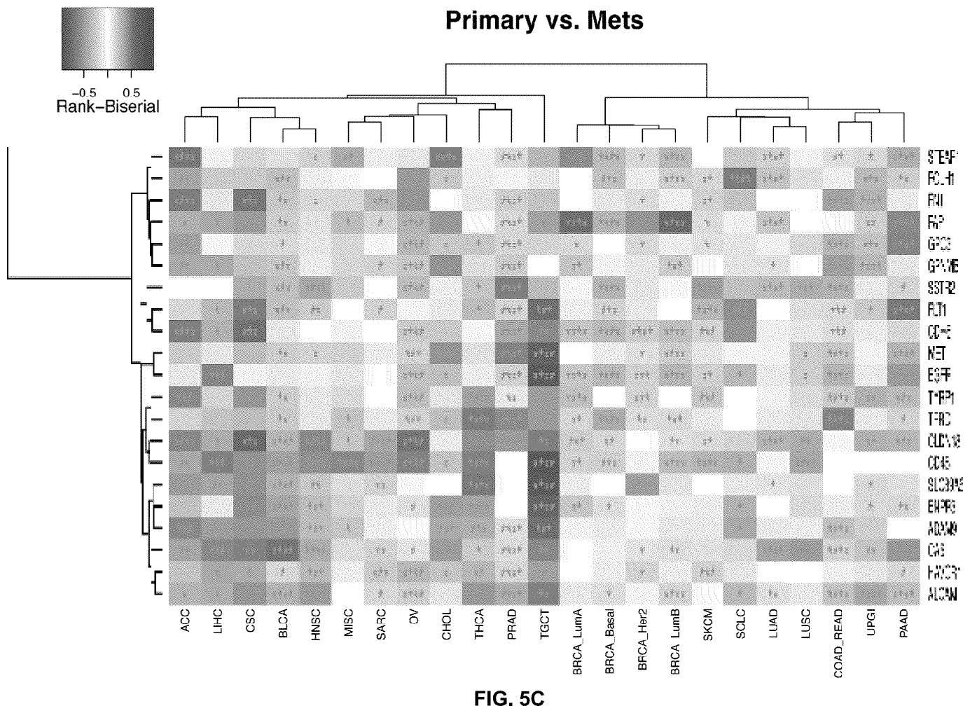

Currently available tumor gene expression datasets that identify cell surface targets are predominantly derived from primary tumor samples, whereas the applications of cell surface directed therapies are primarily approved or investigated in the metastatic setting. This mismatch of primary tumor data with metastatic disease application represents a gap in knowledge. As such, we leveraged our metastasis-enriched gene expression dataset to compare cell surface target expression between primary and metastatic tumors within each tumor type. This analysis demonstrates that many targets had divergent expression between primary and metastatic tumors (FIGS. 5A-5C), which has potential clinical implications regarding therapy selection. For example, NECTIN4, the target of the ADC enfortumab vedotin which is FDA-approved for advanced bladder cancer and under investigation across multiple additional malignancies, has even higher expression in primary compared to metastatic tumors across most tumor types including bladder cancer (BLCA; FIGS. 5A-5C). Conversely, EGFR, the target of a promising BiTE in early-stage trials in pancreatic cancer (4), has higher expression in metastatic compared to primary tumors across many tumor types including pancreatic cancer (PAAD; FIGS. 5A-5C).

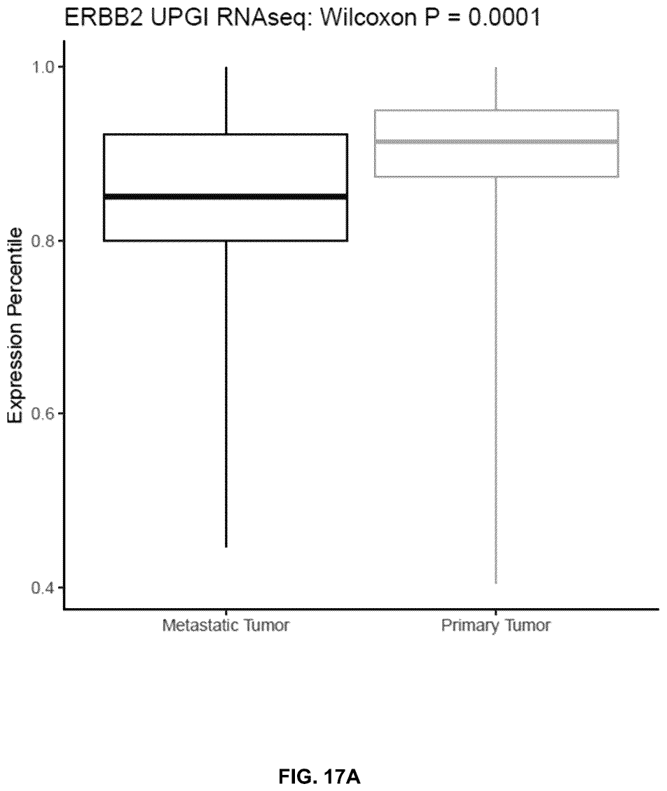

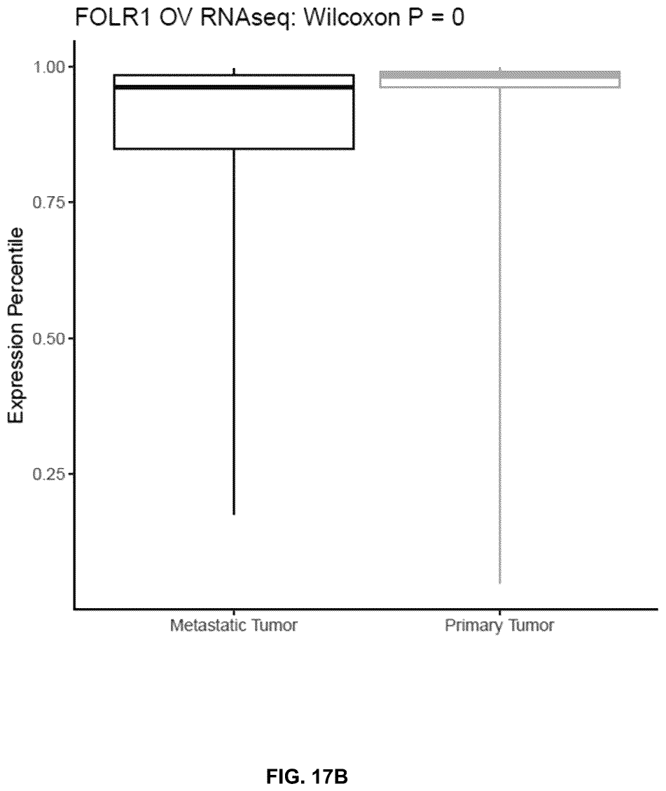

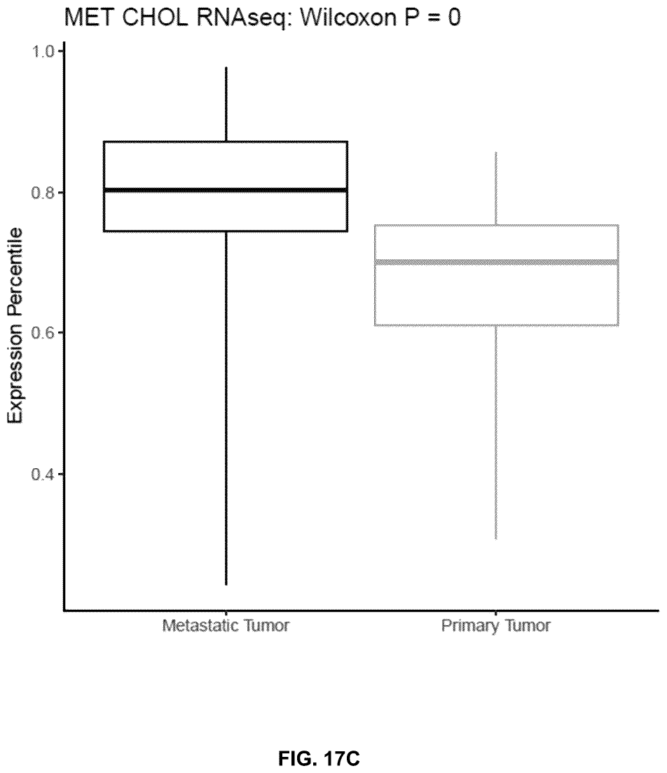

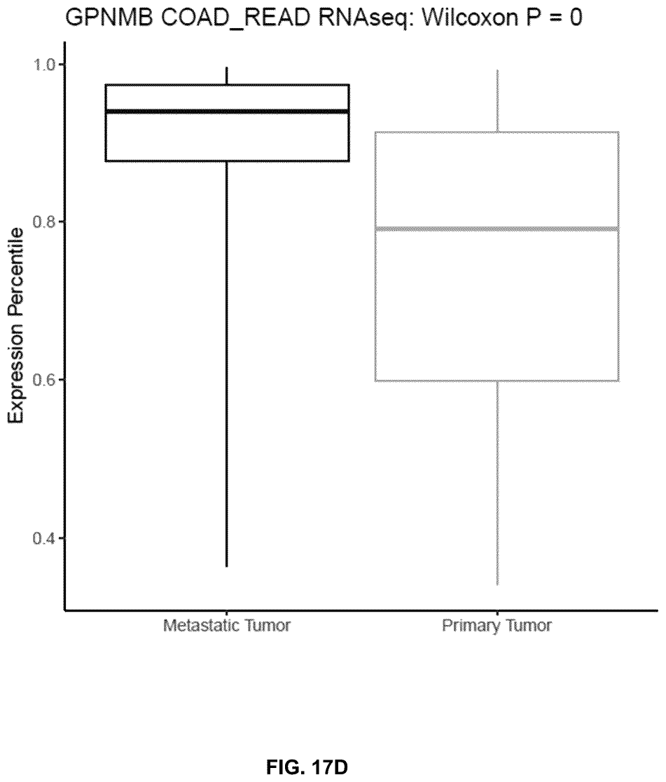

However, many targets had more mixed patterns. ERBB2 is highly expressed in metastatic GI carcinomas (UPGI), for which the ADC fam-trastuzumab deruxtecan is FDA approved, but we find that ERBB2 expression is even higher in primary tumors compared to UPGI metastases (FIGS. 17A-17D). Similarly, we find that FOLR1 has higher expression in primary compared to metastatic ovarian cancers (OV), where the FOLR1-targeting ADC mirvetuximab soravtansine-gynx is FDA approved (FIGS. 17A-17D). These results could be used to guide which cell surface targeted therapies could be moved into earlier disease stages in situations where a clinical rationale exists. Importantly, we also identified target-histology pairs where expression was relatively lower in primary tumors, but higher in metastatic lesions, including MET in cholangiocarcinoma (CHOL) and GPNMB in colorectal (COAD_READ) cancers (FIGS. 17A-17D). All the ADCs for these targets are currently in phase II/III trials in other malignancies, and our results suggest the potential for repositioning these existing ADCs into new clinical indications. Taken together, this highlights the complexity of implementing precision medicine approaches utilizing cell surface targeted therapies, including the importance of evaluating target expression in the specific setting (primary versus metastatic) of proposed use, as well as the potential for drug repositioning across malignancies based on target expression.

### Potential Drug Repositioning Candidates

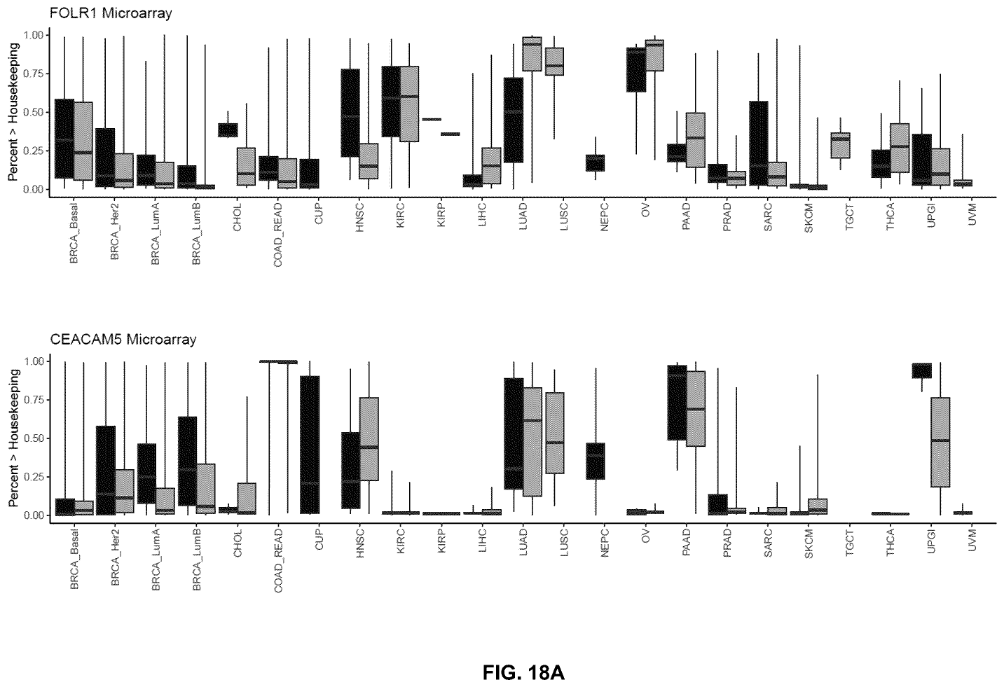

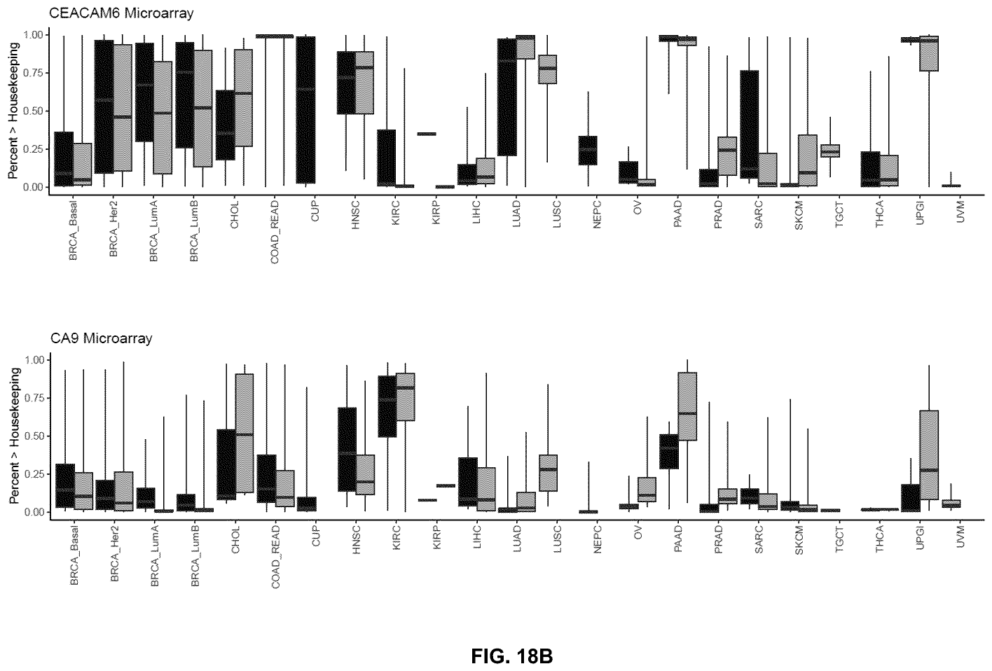

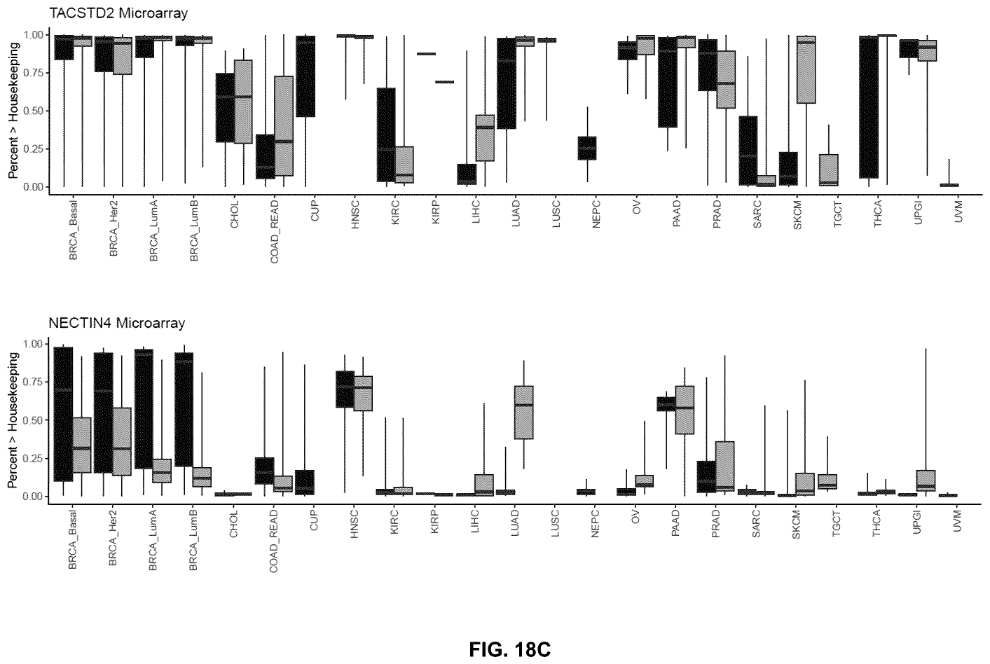

Among the cell surface targets with more tumor-type restricted expression, our pan-cancer approach identifies associations beyond FDA-approved tumor indications, which suggests new avenues for repositioning existing FDA-approved or previously investigated cell surface targeted therapies. As expected, we find that FOLR1 is highly expressed in ovarian cancer (OV), where a FOLR1 ADC is FDA approved, and in NSCLC (LUAD), where FOLR1 ADCs are under investigation. However, we also note high FOLR1 expression in endocervical adenocarcinoma (ECAD), and in clear cell renal cell carcinoma (KIRC) (FIGS. 6A-6C and 18A-18C). CEACAM5/6 expression is elevated in GI malignancies where CEACAM5/6 ADCs are under investigation (COAD_READ, UPGI, SBAD, PAAD), but we also observe high expression in both NSCLC (LUAD) and head and neck squamous cell cancers (HNSC) (FIGS. 6A-6C and 18A-18C). Finally, the CA9 RPT ¹⁷⁷Lu-DPI-4452 is under investigation for RCC (KIRC), pancreatic cancer (PAAD), and colorectal cancer (COAD_READ), all of which are supported by our expression data (FIGS. 6A-6C and 18A-18C). However, other tumor types which may have high expression of CA9 include cholangiocarcinoma (CHOL), cervical squamous cell cancer (CSC), mesothelioma (MESO), and even small bowel adenocarcinoma (SBAD). Our approach also suggests candidate histologies for the repositioning of therapies targeting more ubiquitous cell surface targets. For instance, we find that TACSTD2 (TROP2) is broadly expressed across adenocarcinomas beyond those where TROP2 ADCs are FDA approved or under investigation, including gynecologic (ECAD, OV, UCEC) and GI malignancies (PAAD, UPGI) (FIGS. 6A-6C and 18A-18C). Similarly, we observe that NECTIN4 is not only expressed highly in bladder cancer where it has an FDA-approved ADC, but also across breast cancer subtypes (BRCA), NSCLC (LUAD, LUSC), and some head and neck (HNSC) and cervical (CSC) squamous cell cancers (FIGS. 6A-6C and 18A-18C). These data also reveal that for most cancer types, even when average expression for a cell surface target is low, there are usually at least some tumors with extremely high outlier expression, supporting the rationale for tumor agnostic/basket clinical trials that enroll target-positive tumors regardless of the underlying histology.

Identification of Potential Cell Surface Targets from Clinical RNA-Seq

As more cell surface therapies advance in clinical trials and enter clinical practice, a high-throughput approach to infer target protein expression positivity across cancer types can help guide treatment and clinical trial selection. For instance, patients with advanced metastatic HER2 low breast cancer are eligible for both the TROP2 targeted ADC sacituzumab govitecan and the HER2 targeted ADC fam-trastuzumab deruxtecan (5). Clinicians currently do not have biomarkers to guide the selection of one over the other for an individual patient, which is particularly crucial given emerging data showing poor clinical outcomes with sequential use of these two ADCs (6). As more cell surface targeted trials and therapies become available, this problem will only be exacerbated. Clinical whole-transcriptome RNA-seq is entering clinical practice, with multiple commercial options now available, and represents a potential approach to screen expression of cell surface targets at an individual patient level to address this unmet need. Models, which can be included in the form of a website or web-accessible platform, can be created that allows a percentile score for any cell surface target (calculated for a particular patient's tumor using only their RNA-seq data as described in the methods) to be compared against pan-cancer distributions in order to identify potential highly expressed cell surface targets. We show above that high gene expression accurately predicts high protein expression for most cell surface targets, suggesting that these data could be used for screening potential targets to be confirmed with immunohistochemistry, a requirement for many trials.

### Cell Surface Target Cell Line Model System Matching

To assess the potential for drug repositioning, appropriate cell line models for in vitro and subsequent in vivo investigations are needed. However, these can be challenging to identify, since both expression of the cell surface target and sensitivity to the payload should ideally match that of the clinical tumor population. To address this challenge, we have created a framework for matching the best cell line for each target and tumor type incorporating both these parameters (FIG. 7A). First, we identified cell lines with expression that was ≥95^{th} percentile of expression for each target in the tumor RNA-seq. Next, we correlated every high-purity tumor sample RNA-seq with every cell line, and identified cell lines that were ≥95^{th} percentile of correlation with each tumor type. To validate our approach, we evaluated several specific examples (FIGS. 7B-C). For ERBB2 (Her2) in the Her2 breast cancer samples (BRCA_Her2), most of the top matching cell lines identified were Her2 breast cancer cell lines matched with metastatic Her2 breast cancer samples. For PMEL in the cutaneous melanoma (SKCM) samples, most of the top matching cell lines identified were melanoma cancer cell lines matched with melanoma tumor samples. For FOLH1 (PSMA) in the prostate adenocarcinoma cancer samples, most of the top matching cell lines identified were prostate cancer cell lines matched with prostate cancer tumor samples. Overall, the majority of cell surface targets had at least one cell line matching these criteria (FIG. 7D). In rare tumor types with few/no cell line models, this could be used to find an approximation for in vitro studies. Alternatively, in tumor types with many cell lines available, this could be used to prioritize.

### Single-Cell Heterogeneity of Cell Surface Targets in Tumor Tissue

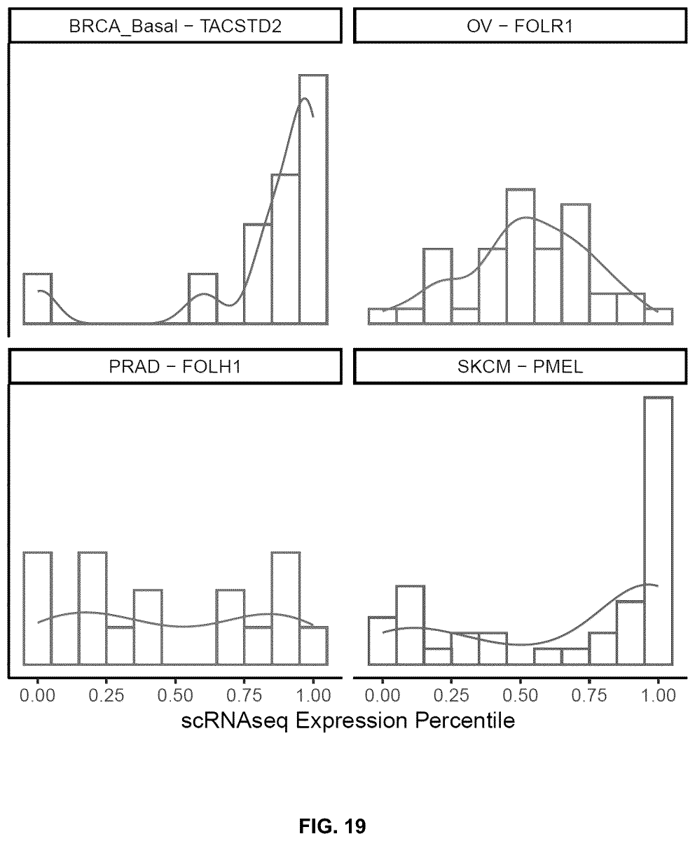

Heterogeneity in tumors is the rule, not the exception, and is a major driver of treatment resistance in metastatic disease. This is an important consideration with respect to payload selection and mechanism of action. Cell surface targeted ADCs and RPTs are designed to deliver a payload to a region surrounding the targeted cells, and thus their efficacy may be less impacted by the heterogeneity of tumors. Conversely, the efficacy of immunomodulatory cell-surface targeted therapies such as CARs and BiTEs may be adversely impacted by heterogeneity of target expression. We utilized scRNA-seq data to assess the degree of variability in cell surface target expression across 484 primary and metastatic tumor samples across a wide range of histologies, focused on the tumor cell component of each sample. Interestingly, while some cell surface targets had relatively uniform expression on most tumor cells in each sample, the majority of cell surface targets demonstrated a large degree of heterogeneity (FIGS. 8A-8D). We next examined several specific FDA-approved indications for cell surface therapies that are not guided by immunohistochemistry (IHC), to evaluate the potential impact of tumor heterogeneity. TACSTD2 (TROP2) was fairly uniformly expressed in basal breast cancer samples, detected in a median of 94% of tumor cells. PMEL was likewise detected in a median of 93% of tumor cells, though some samples exhibited low rates of positivity. In contrast, FOLH1 (PSMA) expression was much more heterogeneous, expressed in a median of 44% of tumor cells in prostate cancer samples, and FOLR1 was expressed in a median of 53% of tumor cells in ovarian cancer samples (FIG. 19). Clinically relevant, this heterogeneity may impact not only treatment response and the development of acquired resistance through selection for cells without target expression, but the consideration of what type of payload might be most efficacious for a particular cell surface target.

### Single-Cell Heterogeneity of Cell Surface Targets in Benign Tissue and Toxicity

While intra-tumoral heterogeneity of target expression could impact tumor treatment responses, differences in target expression in various cellular sub-populations could impact toxicity. Most FDA-approved ADCs have toxicities such as cytopenias, nausea/vomiting, and peripheral neuropathy typically seen with standard cytotoxic chemotherapies and presumed to be due to systemic effects of the cytotoxic chemotherapy payload. However, many of these agents also have toxicities that are not typical of standard cytotoxic agents and could relate to delivery of the payload to normal tissues expressing the cell surface target. For instance, ERBB2 (HER2)-targeted ADC fam-trastuzumab-deruxtecan, FDA approved for HER2-expressing breast cancer and NSCLC, has a well described risk of interstitial lung disease (ILD), which can be seen in up to 10% of patients, and can be fatal (7). This is also seen with ERBB3 (HER3) (8, 9) and TACSTD2 (TROP2) targeted (10) deruxtecan conjugates. However, the mechanistic contribution of payload type versus cell surface target expression remains poorly understood. We evaluated ERBB2, ERBB3 and TACSTD2 expression in scRNAseq of the normal lung in our dataset and identified enrichment of cells expressing all three targets among AT1, ciliated, and club cell populations, in contrast to other cell types (FIG. 8E). As this pulmonary toxicity is not seen with govitecan conjugates targeting TROP2 (sacituzumab govitecan) (11, 12) or HER2 (disitamab vedotin) (13), the ILD phenotype is likely driven by the specific combination of the deruxtecan payload and target expression on these lung cell populations. We also leveraged our dataset to investigate a unique toxicity of enfortumab vedotin, an FDA-approved NECTIN4-targeting ADC, which carries a risk of severe hyperglycemia, seen in up to 8% of patients (14). This is also seen with ladiratuzumab vedotin targeting SLC39A6 (15) and glembatumumab vedotin targeting GPNMB (16). However, it is not seen with any other solid tumor vedotin-conjugate ADCs with published toxicity data including telisotuzumab vedotin (MET) (17), tisotumab vedotin (tissue factor) (18), disitamab vedotin (HER2) (13), indusatumab vedotin (GUCY2C) (19) or samrotamab vedotin (LRRC15) (20). When we investigated expression of these targets in scRNAseq of the normal pancreas, we found that target detection on pancreatic alpha cells, but not other pancreatic cell types, correlated with the presence or absence of hyperglycemia as a toxicity across these vedotin-conjugate therapies (FIG. 8F). As pancreatic alpha cells play a key role in glucagon secretion, this suggests that on target binding and/or payload release of the respective ADCs to these cells may lead to glucagon release to drive the acute hyperglycemia seen with these agents.

### Combinations of Cell Surface Targets can Improve Cancer Specificity

Historically, cell surface targeted therapies have relied on monovalent antibodies. However, combinatorial approaches can potentially improve cancer specificity and decrease normal tissue exposure. Emerging approaches allow for bivalent combinations of cell surface targets. Several bispecific ADCs and CAR-Ts are under investigation in clinical trials. However, identifying appropriate combinations empirically presents challenges. We therefore sought to evaluate all combinations of two clinical cell surface targets in their combinatorial differential expression between cancers and normal tissues. To do this, we created logistic regression models for every pair of cell surface targets comparing each cancer type and each normal tissue type. We then only retained pairs where 1) expression was higher in cancer vs. normal and the combination had good discriminative power with an F1-score >0.95, and 2) each individual gene was contributing independently, with both having Wald test p-values (corrected for multiple testing) <0.05. Overall, only a small minority of cancer-normal tissue pairs across cancer types met these stringent criteria (FIG. 9A), but these represent potential promising combinatorial cell surface strategies. Several examples of bivalent combinations of clinical cell surface targets demonstrate a markedly improved ability to stratify cancers from normal tissues (FIG. 9B-9D), suggesting that this approach may improve the therapeutic window and selectivity of agents directed against more ubiquitously expressed targets.

### Discussion

While most cell surface targeted therapies are undergoing initial development in metastatic disease, prior studies evaluating cell surface target expression across tumor types have predominantly focused on primary tumor target expression, due to the more limited availability of metastatic tumor biopsies. We have directly addressed this key gap through the curation of a large pan-cancer and normal tissue gene expression dataset including over 6000 metastatic tumors. To our knowledge, this represents the largest integrated dataset of metastatic tumor tissue gene expression focused on cell surface target expression reported to date. Leveraging this unique resource, we are able to provide a comprehensive overview of cell surface target gene expression in primary and metastatic solid tumors with a focus on cell surface targets with existing FDA-approved or investigational cell surface targeted therapies. Our analysis demonstrated a number of targets with differential expression between primary and metastatic disease with the potential to guide avenues of future clinical development for therapies directed against these targets. Additionally, we identified multiple opportunities for repositioning existing FDA-approved and investigational cell surface targeted therapies to new tumor types, as well as an approach to identifying appropriate in vitro models for drug repositioning.

Leveraging tumor scRNAseq data, we found that intra-tumoral heterogeneity varied across surveyed targets, including those with FDA-approved indications. ADCs and RPTs likely provide some level of bystander effect that can potentially overcome heterogeneity. However, higher heterogeneity of target expression could still lead to selection for non-target expressing tumor cells as a mechanism of acquired resistance to these therapy classes. CAR-Ts and BiTEs, which drive immune responses to the target antigen, may be more sensitive to target expression variability within a tumor. It is notable that to date these agents have been most successful in hematologic malignancies subject to ubiquitous and obligate expression of the cell surface target. These principles also apply to normal tissues, where selective enrichment of a target in a critical cell population could lead to clinically relevant toxicities from particular target/payload combinations, as we have identified in both normal lung and pancreas through an analysis of normal tissue scRNAseq. Taken together, our findings emphasize the importance of incorporating single-cell RNA-seq in the identification of potential cell surface targets, as well as in the understanding of toxicities that emerge.

Our study is not without limitations. Due to well-established potential for dropout in single cell RNA sequencing, the scRNAseq data were binarized (21-23); while the correlation between these binarized values and protein expression is not well understood, these results do illustrate the influence of heterogeneity of target gene expression at least at the transcriptional level. The aggregation of bulk RNA sequencing data across multiple studies comprising samples of varying quality and techniques is not equivalent to a single uniform dataset. Given the overarching goals of this study, we prioritized broader inclusion of available samples to improve our sampling of rare cancer types. Nonetheless, the high expression of cell surface targets in the tumor types which we would expect are re-assuring. Additionally, we were able to validate in a set of matched samples that high gene expression using this approach accurately predicts high protein expression for our panel of cell surface targets. However, the degree of protein expression required for effective cell-surface targeted therapy is unknown and the efficacy of CST ultimately depends not only on target engagement but also linker-dependent payload delivery and tumor payload sensitivity. For instance, in the phase I/II pan-cancer trial of sacituzumab govitecan, where hormone receptor positive and triple negative breast cancer (BRCA) demonstrated clinical benefit rates of 44.4% and 45.4%, respectively, other tumor types identified in our analysis with high TROP2 expression had more variable clinical benefit rates, ranging from 0% for pancreatic adenocarcinoma (PAAD) to 21.1% for esophageal carcinoma (UPGI) to 44.4% for endometrial cancer (UCEC) (24). Similarly, recently reported phase II studies of the NECTIN4 ADC enfortumab vedotin have demonstrated anti-tumor activity in tumor types identified in our analysis as having high NECTIN4 expression, however response rates were variable and in all cases lower than the 40-50% overall response rate seen in urothelial cancer (25) (BLCA). In triple negative and hormone receptor positive breast cancer, anti-tumor activity was demonstrated with overall response rates of 19% and 15.6%, but did not meet prespecified thresholds to continue with further development (26). In NSCLC, anti-tumor activity was seen in the adenocarcinoma (LUAD) cohort, but not in the squamous (LUSC) cohort (27). Taken together, this highlights the complexity of predicting tumor response and resistance to CSTs, though the presence or absence of target expression will remain an important component of this process.

With the advent of bi-specific antibodies and beyond, combinatorial approaches are beginning to gain traction, and offer a potential way to improve the therapeutic ratio of cell surface targeted therapies, increasing tumor specificity and reducing toxicities related to on-target gene expression in normal tissues. However, selection of appropriate target combinations empirically is exceedingly challenging, and we describe a computational methodology to identify potential combinations with the highest likelihood of efficacy while minimizing on target toxicities. As more advanced cell surface targeting technologies move forward, particularly those leveraging immunomodulatory payloads, we hope that this resource could be used to identify unappreciated combinations, such as the ones we illustrate.

Finally, as clinical indications for cell surface targeted therapies expand, precision oncology biomarkers for selection of the most appropriate therapy for an individual patient are needed. RNA-seq in addition to DNA sequencing is becoming more mainstream in clinical practice, with multiple commercial options. This poses a challenge for oncologists to interpret, as clinical indications have not yet begun to incorporate RNA expression of specific targets. While RNA and protein levels are moderately correlated, it is still not a perfect surrogate. However, RNA-seq could be used to screen for potential highly expressed surface targets, which could then be confirmed by IHC. A high-throughput method of identifying potential cell surface targets will become increasingly important as more of these therapies enter clinical trials and practice, as it will not be practical to stain for all possible targets. Herein, we have also provided a reference of the distributions of every cell surface target, which could be used as a resource by oncologists or molecular tumor boards. Since the percentile for any target can be calculated using only an individual patient's RNA-seq profile, these data could be used to help guide the subset of targets to confirm with IHC.

### Methods

### Sex as a Biological Variable

Our study studied tumor samples from both male and female patients in publicly available gene expression datasets.

### Clinical Landscape of Cell-Surface Protein Targets in Adult Solid Tumors

We utilized ClinicalTrials.gov to compile a comprehensive list of interventional clinical trials (as of Oct. 31, 2023) on various targeted cancer therapies for adult solid tumors. The targeted therapies include antibody-drug conjugates (ADC), bispecific T-cell engagers (BiTE), chimeric antigen receptor T-cell (CAR-T), other CAR variants such as CAR-natural killer cells (CAR-NK), CAR-dendritic cells (CAR-DC), CAR-macrophage (CAR-M), and radiopharmaceutical therapy (RPT). We manually curated the cell surface protein targets in each trial based on information from trial details and related publications. For phase annotation, we only considered the most advanced phase in the case of multi-phase trials. For instance, trials in phases 112 were categorized as phase 2, while those in phases 213 were considered phase 3 trials. Targets were excluded from subsequent analyses if there was not a specific gene coding for the protein, the absence of these genes in all datasets, or as part of a NOT-gate CAR-T design. In addition, targets that had only a single ended trial without any additional trials were excluded.

### Bulk Tumor RNAseq & Microarray Data

We have compiled gene expression data from a large collection of RNAseq (N=52) and Microarray (N=85) studies. These studies encompass various cancer types, normal tissues, and cell lines. The datasets were downloaded from multiple sources, including the Gene Expression Omnibus (GEO), cBioPortal, and supplementary files from individual publications, among others. To standardize gene symbols across various versions of gene annotations used in different studies, we converted different gene identifiers (Ensembl gene ID, Entrez gene ID, RefSeq gene ID, Gene Atlas, etc) to official HGNC gene symbol using the R packages biomaRt and AnnotationDBi. Only protein-coding genes with valid HUGO gene symbols were retained. For microarray data, the corresponding probe annotation file for a given platform was downloaded using the R package annotate. In cases where multiple probes mapped to the same gene, we selected the probe with the highest average expression across samples.

Clinical data was primarily downloaded using the R package GEOquery, unless otherwise provided. We extracted relevant clinical information, including cancer type and primary or metastatic tumor. Cancer types were annotated in accordance with the TCGA cancer type abbreviations (Table 1). We performed subtyping analyses on breast cancer samples and cancer cell lines into luminal A, luminal B, her2-rich, and basal subtypes using R package AIMS. In cases where the ‘normal-like’ subtype was initially assigned, we selected the second-highest scoring subtype as the subtype assignment.

TABLE 1
TCGA cancer type abbreviations.

| Abbreviation | Cancer Type |
|---|---|
| ACC | Adrenocortical Carcinoma |
| ASC | Anal Squamous Cell Carcinoma |
| BLCA | Bladder Urothelial Carcinoma |
| BRCA_Basal | Breast Invasive Carcinoma (Basal Subtype) |
| BRCA_Her2 | Breast Invasive Carcinoma (Her2 Subtype) |
| BRCA_LumA | Breast Invasive Carcinoma (Luminal A Subtype) |
| BRCA_LumB | Breast Invasive Carcinoma (Luminal B Subtype) |
| CHOL | Cholangiocarcinoma |
| CRC | Colorectal Adenocarcinoma |
| CSC | Cervical Squamous Cell Carcinoma |
| CUP | Cancer of Unknown Primary |
| DLBC | Diffuse Large B-Cell Lymphoma |
| ECAD | Endocervical Adenocarcinoma |
| ESSC | Esophagus Squamous Cell Carcinoma |
| FLC | Fibrolamellar Carcinoma |
| GBM | Glioblastoma Multiforme |
| GIST | Gastrointestinal Stromal Tumor |
| HNACC | Adenoid Cystic Carcinoma |
| HNSC | Head and Neck Squamous Cell Carcinoma |
| KICH | Kidney Chromophobe |
| KIRC | Kidney Renal Clear Cell Carcinoma |
| KIRP | Kidney Renal Papillary Cell Carcinoma |
| LAML | Acute Myeloid Leukemia |
| LGG | Brain Lower Grade Glioma |
| LIHC | Liver Hepatocellular Carcinoma |
| LUAD | Lung Adenocarcinoma |
| LUSC | Lung Squamous Cell Carcinoma |
| MESO | Mesothelioma |
| MISC | Miscellaneous |
| NEPC | Prostate Neuroendocrine Carcinoma |
| OCCC | Clear Cell Ovarian Cancer |
| OV | Ovarian Serous Cystadenocarcinoma |
| PAAD | Pancreatic Adenocarcinoma |
| PCPG | Pheochromocytoma and Paraganglioma |
| PRAD | Prostate Adenocarcinoma |
| SARC | Sarcoma |
| SBAD | Small Bowel Adenocarcinoma |
| SCLC | Small Cell Lung Cancer |
| SCNE | Other Small Cell or Neuroendocrine Tumor |
| SKCM | Skin Cutaneous Melanoma |
| TGCT | Testicular Germ Cell Tumor |
| THCA | Thyroid Carcinoma |
| THYM | Thymoma |
| UCEC | Uterine Corpus Endometrial Carcinoma |
| UCS | Uterine Carcinosarcoma |
| UPGI | Esophagus/Stomach Adenocarcinoma |
| UVM | Uveal Melanoma |

We integrated all 52 RNAseq studies, retaining only the 17,560 protein-coding genes common to all. The resulting combined RNAseq dataset comprises these genes across 25,582 samples, including 7,927 normal tissue samples, 12,398 primary tumors, 3,807 metastatic tumors, and 1,450 cell lines. We implemented a sample-wise rank transformation (‘ties.method=“random”’), assigning ranks from 1 to 17,560, with higher ranks indicating increased gene expression. Subsequently, we converted these ranks to percentiles by dividing each rank by the maximum of 17,560, thereby standardizing gene expression to a range from 0 to 1 for each gene in every sample.

Due to the variable number of genes across the microarray studies, we combined all 85 datasets using a union set of protein-coding genes identified in each study in order to preserve maximum information. This yielded a combined microarray dataset containing 19,200 genes across 6,785 samples, including 4,468 primary tumors and 2,317 metastatic tumors. Similar to RNA-seq data, we implemented a sample-wise rank transformation (‘ties.method=“random”’).

### Housekeeping Gene References

We use housekeeping genes as references to assess the expression levels of target genes (21). We calculated median RNA expression distribution of housekeeping genes in each RNAseq study included in the study. Within each RNA-seq study, the 1st, 50th, and 90th percentiles of all housekeeping gene expressions were calculated. The median values of these percentiles across the studies were used as thresholds of low, medium, and high gene expression levels.

Because of variable numbers of genes on the disparate microarray platforms used, in order to make values comparable across samples from different studies, we then instead examined the percentage of housekeeping genes with lower expression than each gene. For example, a gene with expression greater than 90% of housekeeping genes in a given sample would be assigned a value of 0.9. This method is relatively insensitive to missing genes given the large number of housekeeping genes.

### Single-Cell RNAseq Data

To further investigate the heterogeneity of target expression in tumors and normal tissues, we utilized two scRNA-seq datasets. The first dataset is the Human Cell Landscape, a pan-tissue scRNA-seq dataset (22), which includes 57 normal tissue samples across 36 normal tissue types, with replicates in several normal tissues. The second dataset is the Curated Cancer Cell Atlas of Tumors, a comprehensive curated pan-cancer scRNA-seq dataset (23). Only datasets on adult solid tumors were used, which include 558 tumor samples across 27 cancer types and 53 studies. The primary vs. metastatic origin of each sample was not available. The scRNAseq cell-by-gene count matrices and cell annotation files were integrated by aligning cell IDs, and the scRNA-seq count matrix was converted into a binary matrix to indicate gene expression's measurability or detectability by setting any non-zero expression value as 1(24-26). Our analysis was exclusively focused on the cell surface protein targets identified in the clinical trials.

### RNA and Protein Expression

To investigate RNA-protein expression correlation, we utilized paired RNA and protein expression data from three different sources, including normal tissues from the HPA (3) (N=29), primary tumors from the CPTAC (pdc.cancer.gov/pdc/cptac-pancancer; N=1,075), and cell lines from the SCMP (2) (N=942). We converted various gene identifiers to HGNC gene symbols, retaining only those protein-coding genes common to both RNA and protein expression datasets. Both RNA and protein datasets underwent percentile rank transformation. High expression of a gene was defined per-sample as being >90% of housekeeping genes.

### Cell Line Models

We selected cell line models that are most representative for a specific cancer type based on strong correlation based on transcriptomic profiling and high cell surface protein target expression. Spearman's correlation was calculated for each tumor sample exhibiting high tumor purity (≥0.6, as recommended by the TCGA consortium) against each cell line (solid tumors with clearly defined cancer type annotations only). Tumor purity was estimated using the ESTIMATE algorithm (27) via the R package tidyestimate.

### Statistics

Statistical tests used are indicated in each figure legend. A p-value of 0.05 was used to determine significance. All tests are two-sided.

### Data Availability

Single-cell RNA-seq data were obtained from db.cngb.org/HCL/and weizmann.ac.il/sites/3CA/. All data are available per these sources and repositories. Supporting data values are provided in the respective file. All other code and data sharing requests will require institutional review and a data sharing agreement.

### REFERENCES

1. Bosi C, Bartha A, Galbardi B, Notini G, Naldini M M, Licata L, et al. Pan-cancer analysis of antibody-drug conjugate targets and putative predictors of treatment response. Eur J Cancer. 2023; 195:113379.
2. Goncalves E, Poulos R C, Cai Z, Barthorpe S, Manda S S, Lucas N, et al. Pan-cancer proteomic map of 949 human cell lines. Cancer Cell. 2022; 40(8):835-49 e8.
3. Wang D, Eraslan B, Wieland T, Hallstrom B, Hopf T, Zolg D P, et al. A deep proteome and transcriptome abundance atlas of 29 healthy human tissues. Mol Syst Biol. 2019; 15(2):e8503.
4. Lum L G, Thakur A, Choi M, Deol A, Kondadasula V, Schalk D, et al. Clinical and immune responses to anti-CD3 x anti-EGFR bispecific antibody armed activated T cells (EGFR BATs) in pancreatic cancer patients. Oncoimmunology. 2020; 9(1):1773201.
5. National Comprehensive Cancer Network. Breast Cancer (Version 1.2024). Accessed Feb. 25, 2024.
6. Fenton M A, Tarantino P, and Graff S L. Sequencing Antibody Drug Conjugates in Breast Cancer: Exploring Future Roles. Curr Oncol. 2023; 30(12):10211-23.
7. Daiichi Sankyo. Enhertu (fam-trastuzumab deruxtecan-nxki) [package insert]. Updated December 2019 Accessed Feb. 25, 2024.
8. Krop I E, Masuda N, Mukohara T, Takahashi S, Nakayama T, Inoue K, et al. Patritumab Deruxtecan (HER3-DXd), a Human Epidermal Growth Factor Receptor 3-Directed Antibody-Drug Conjugate, in Patients With Previously Treated Human Epidermal Growth Factor Receptor 3-Expressing Metastatic Breast Cancer: A Multicenter, Phase I/II Trial. J Clin Oncol. 2023; 41(36):5550-60.
9. Yu H A, Goto Y, Hayashi H, Felip E, Chih-Hsin Yang J, Reck M, et al. HERTHENA-Lung01, a Phase II Trial of Patritumab Deruxtecan (HER3-DXd) in Epidermal Growth Factor Receptor-Mutated Non-Small-Cell Lung Cancer After Epidermal Growth Factor Receptor Tyrosine Kinase Inhibitor Therapy and Platinum-Based Chemotherapy. J Clin Oncol. 2023; 41(35):5363-75.
10. Shimizu T, Sands J, Yoh K, Spira A, Garon E B, Kitazono S, et al. First-in-Human, Phase I Dose-Escalation and Dose-Expansion Study of Trophoblast Cell-Surface Antigen 2-Directed Antibody-Drug Conjugate Datopotamab Deruxtecan in Non-Small-Cell Lung Cancer: TROPION-PanTumor01. J Clin Oncol. 2023; 41(29):4678-87.
11. Bardia A, Hurvitz S A, Tolaney S M, Loirat D, Punie K, Oliveira M, et al. Sacituzumab Govitecan in Metastatic Triple-Negative Breast Cancer. N Engl J Med. 2021; 384(16):1529-41.
12. Gilead Sciences. Trodelvy (sacituzumab govitecan-hziy) [package insert]. Updated February 2023 Accessed Feb. 25, 2024.
13. Sheng X, Wang L, He Z, Shi Y, Luo H, Han W, et al. Efficacy and Safety of Disitamab Vedotin in Patients With Human Epidermal Growth Factor Receptor 2-Positive Locally Advanced or Metastatic Urothelial Carcinoma: A Combined Analysis of Two Phase II Clinical Trials. J Clin Oncol. 2023:JC02202912.
14. Astellas Pharma US. Padcev (enfortumab vedotin-ejfv) [package insert]. Updated December 2019 Accessed Feb. 25, 2024.
15. Tsai M, Han H S, Montero A J, Tkaczuk K H, Assad H, Pusztai L, et al. 259P Weekly ladiratuzumab vedotin monotherapy for metastatic triple-negative breast cancer. Annals of Oncology. 2021; 32:S474-S5.
16. Ott P A, Hamid O, Pavlick A C, Kluger H, Kim K B, Boasberg P D, et al. Phase I/II study of the antibody-drug conjugate glembatumumab vedotin in patients with advanced melanoma. J Clin Oncol. 2014; 32(32):3659-66.
17. Camidge D R, Barlesi F, Goldman J W, Morgensztern D, Heist R, Vokes E, et al. Phase Ib Study of Telisotuzumab Vedotin in Combination With Erlotinib in Patients With c-Met Protein-Expressing Non-Small-Cell Lung Cancer. J Clin Oncol. 2023; 41(5):1105-15.
18. Seagen. Tivdak (tisotumab vedotin-tftv) [package insert]. Updated September 2021 Accessed Feb. 24, 2025.
19. Almhanna K, Wright D, Mercade T M, Van Laethem J L, Gracian A C, Guillen-Ponce C, et al. A phase II study of antibody-drug conjugate, TAK-264 (MLN0264) in previously treated patients with advanced or metastatic pancreatic adenocarcinoma expressing guanylyl cyclase C. Invest New Drugs. 2017; 35(5):634-41.
20. Demetri G D, Luke J J, Hollebecque A, Powderly J D, 2nd, Spira A I, Subbiah V, et al. First-in-Human Phase I Study of ABBV-085, an Antibody-Drug Conjugate Targeting LRRC15, in Sarcomas and Other Advanced Solid Tumors. Clin Cancer Res. 2021; 27(13):3556-66.
21. Eisenberg E, and Levanon E Y Human housekeeping genes, revisited. Trends Genet. 2013; 29(10):569-74.
22. Han X, Zhou Z, Fei L, Sun H, Wang R, Chen Y, et al. Construction of a human cell landscape at single-cell level. Nature. 2020; 581(7808):303-9.
23. Gavish A, Tyler M, Greenwald A C, Hoefflin R, Simkin D, Tschernichovsky R, et al. Hallmarks of transcriptional intratumour heterogeneity across a thousand tumours. Nature. 2023; 618(7965):598-606.
24. Bouland G A, Mahfouz A, and Reinders M J T. Differential analysis of binarized single-cell RNA sequencing data captures biological variation. NAR Genom Bioinform. 2021; 3(4):lqab118.
25. Bouland G A, Mahfouz A, and Reinders M J T. Consequences and opportunities arising due to sparser single-cell RNA-seq datasets. Genome Biol. 2023; 24(1):86.
26. Qiu P. Embracing the dropouts in single-cell RNA-seq analysis. Nat Commun. 2020; 11(1):1169.
27. Yoshihara K, Shahmoradgoli M, Martinez E, Vegesna R, Kim H, Torres-Garcia W, et al. Inferring tumour purity and stromal and immune cell admixture from expression data. Nat Commun. 2013; 4:2612.

### Example 2. Matching Patients with Cell Surface Targeted Clinical Trials Using Large Language Models

### Summary

Cell surface-targeted therapies (CSTs) represent a rapidly expanding class of cancer treatments with high specificity and reduced toxicity. However, matching patients who express specific targets to CST clinical trials remains a major challenge due to the complexity of eligibility criteria, the diversity of targets, and the lack of centralized, up-to-date trial databases. This limits patient access to potentially beneficial therapies and contributes to delayed and/or poor trial accrual.

We developed a large language model (LLM)-driven pipeline to automate the identification and annotation of CST clinical trials. Using a two-pronged approach, we prompted LLMs to extract target information from ClinicalTrials.gov and the NCI Drug Database. We benchmarked eight LLMs, including GPT-4o and several open-source models, against a manually curated dataset of 814 CST trials. We evaluated model performance at both the target and trial levels and analyzed sources of error. Additionally, we assessed LLMs' ability to predict cell surface localization of genes, important for novel target identification.

GPT-4o achieved the highest accuracy in both identifying CST trials (97%) as well as identifying the targets of those CSTs (89.5%). Combining data sources improved performance, and model accuracy increased as the trial phase progressed. Most errors stemmed from vague therapy descriptions or string-matching issues. Geographically, the model matched 94% of U.S. trials and >95% of trials globally, with exceptions in China and New Zealand. In predicting protein cell surface localization, Gemma 3:27b and MedLlamav3 were able to correctly label all known clinical cell surface targets, though models had more variable performance beyond the most well-known CSTs.

Our LLM-based approach enables real-time, automated matching of patients to CST clinical trials, potentially expanding trial accessibility and improving enrollment efficiency. Errors were uncommon, and performance is poised to improve as LLMs evolve. Optimizing patient-trial matching for CSTs can improve both the odds of patient benefit as well as the odds of a successful trial.

### Introduction

Clinical trials form the backbone of clinical cancer research and are required to investigate new therapies and set new standards of care. Notably, early patient accrual is associated with overall accrual¹, emphasizing the importance of identifying the right patients for enrollment. Furthermore, it is estimated that only a small minority (on the order of 2-4%) of patients ultimately participate in cancer-related clinical trials, despite a large majority of Americans having a favorable view of participating in cancer-related clinical trials²⁻⁴. There are numerous reasons leading to difficulty enrolling patients onto clinical trials. One meta-analysis showed that half of patients simply did not have a clinical trial open at the treating institution³. Other factors are associated with the knowledge, labor, and costs related to accruing patients in general. One study reported that two of the main contributors to difficult patient enrollment were knowledge of clinical trial availability and complexity of the clinical trial documentation and enrollment criteria⁵. Additional investigations into clinical trial enrollment logistics also emphasize the cost and effort required to successfully accrue a patient, with screening a single patient for eligibility requiring 3.4-8.8 hours and $129-336, depending on patient-specific factors⁶. Ultimately, these obstacles to trial enrollment result in approximately 40% of clinical trials failing to meet accrual goals⁷. An analysis of National Clinical Trials Network (NCTN) sponsored trials has further shown that 18% of these trials were either closed or were at less than 50% of target accrual three years or more after initiation⁸. Difficulty in enrolling patients causes delays in the development of new therapies and deprives patients who would have qualified for beneficial therapies.

Cell surface-targeting therapies (CSTs) represent an emerging class of highly effective drugs for both hematologic and solid tumor malignancies, with several FDA-approved therapies in use clinically. The potential benefit of CSTs over conventional cytotoxic therapies is increased cancer specificity and less toxicity. There are several categories of therapies that have been developed, including antibody-drug conjugates (ADCs), radiopharmaceuticals (RPTs), as well as immunomodulatory therapies such as chimeric antigen receptor T cells (CAR-Ts), CAR natural killer cells (CAR-NKs), CAR dendritic cells (CAR-DCs), CAR macrophages (CAR-Ms), and bispecific T cell engagers (BiTEs). A unique eligibility requirement for many CST trials is expression of the target, often confirmed via imaging or pathology. In many cases, the degree of expression of the target cell surface marker correlates with response⁹⁻¹⁹, though this is not universal²⁰. However, most clinical trials of novel CSTs require cell surface marker expression, either directly or indirectly (e.g. by selecting a disease state where expression is ubiquitous). High-throughput methods exist for identifying and confirming potential targets expressed in a patient²¹⁻³³. However, once suitable targets are identified, there is no easy way to identify potential trials outside of the treating institution, which often limits therapies that are available to patients. This is particularly difficult for CSTs given the large number of potential targets. Many trials are also multi-cancer and predicated solely on target expression. Thus, even disease-site experts are unlikely to be familiar with all available CST trials for every possible target. Given the increasing numbers of CST clinical trials and the difficulty in accruing patients to oncology clinical trials in general, it is critical to develop a tool that can match patients to trials more broadly.

The recent development and expansion of large language models (LLMs) has made the process of parsing and interpreting large amounts of text much more efficient. LLMs have been utilized to match patients to clinical trials in an effort to make patient-trial matching more efficient and less costly³⁴. However, the oncology-focused approaches lack data on their performance specifically for CSTs³⁵. Existing approaches are largely centered on parsing text from a patient's electronic health record (EHR) and matching this to explicit clinical trial criteria^{7,35-37}. We developed a unique LLM-based approach which integrates disparate sources of CST and trial information and created a unified database that can be updated in real-time as new trials open. We then validated and compared the performance of eight different LLMs in a human-curated set of 814 CST trials²¹. Our tool is capable of accurately identifying suitable CST trials for specific targets, and can be used to both increase enrollment on these trials as well as better match patients with trials from which they are most likely to benefit.

### Results

### LLM Generation of a CST Clinical Trials Database

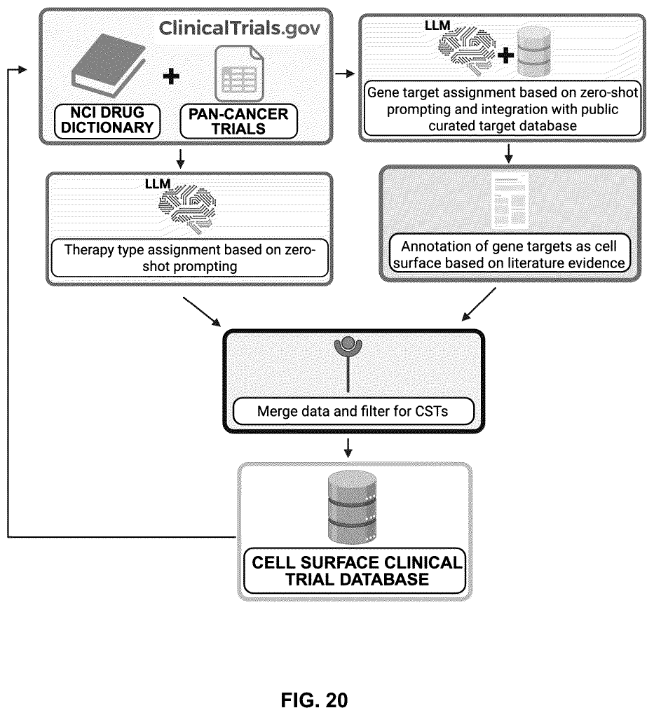

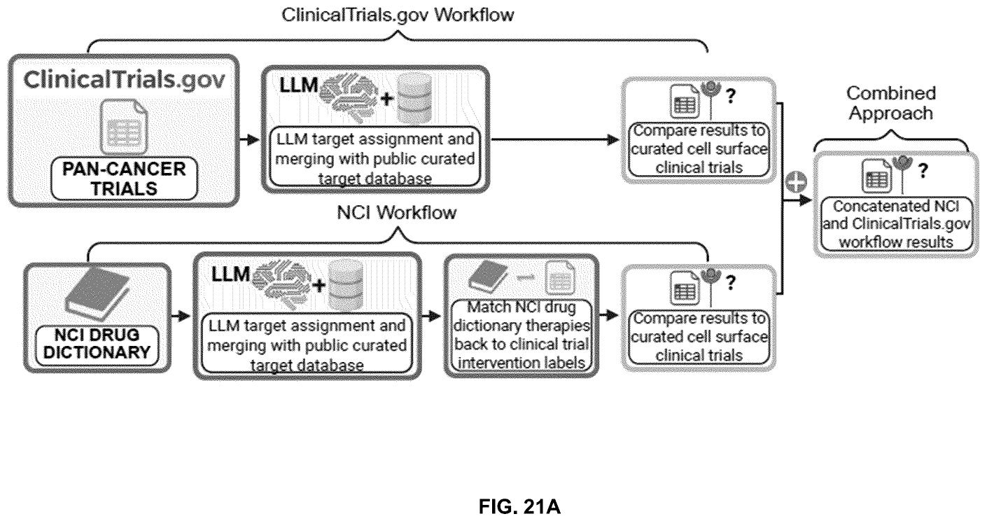

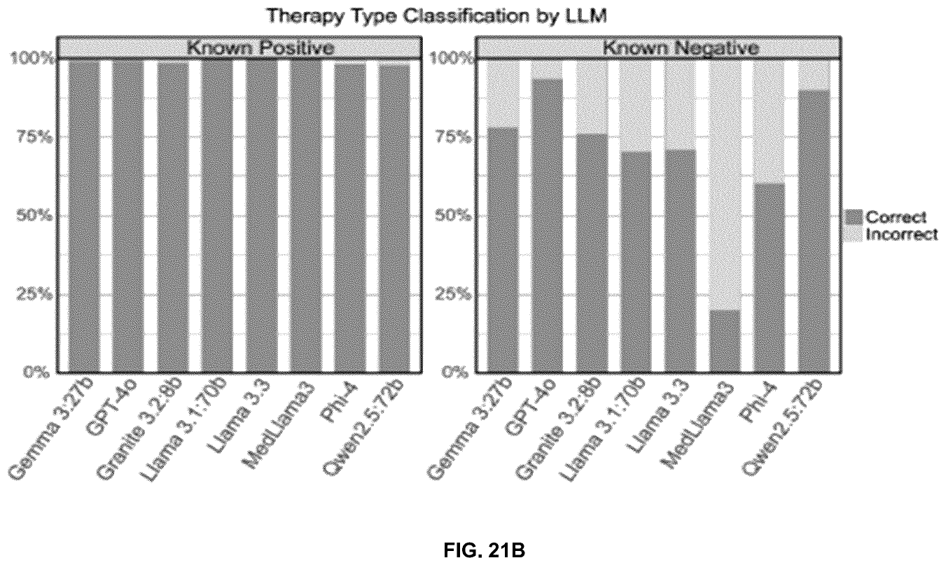

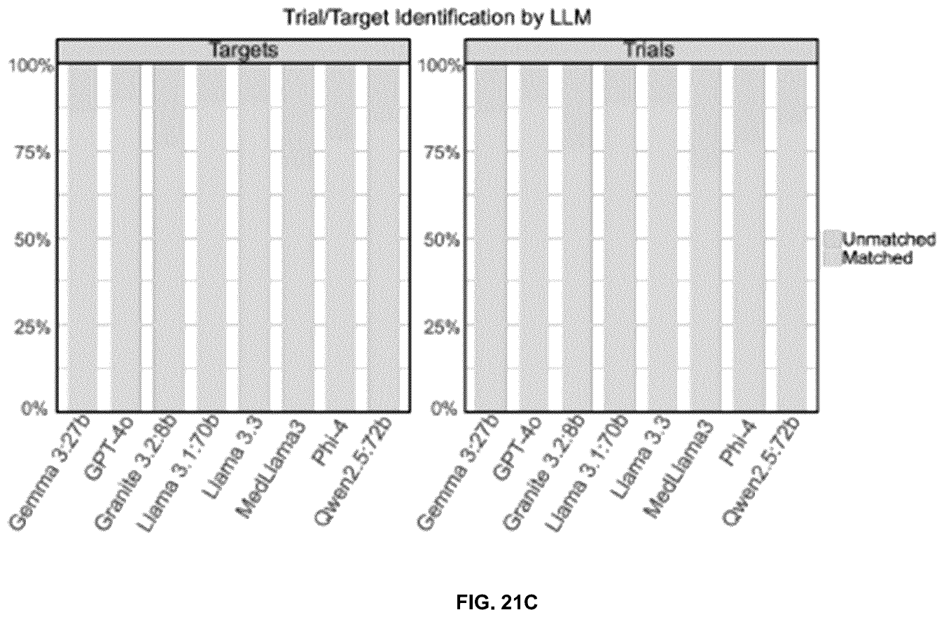

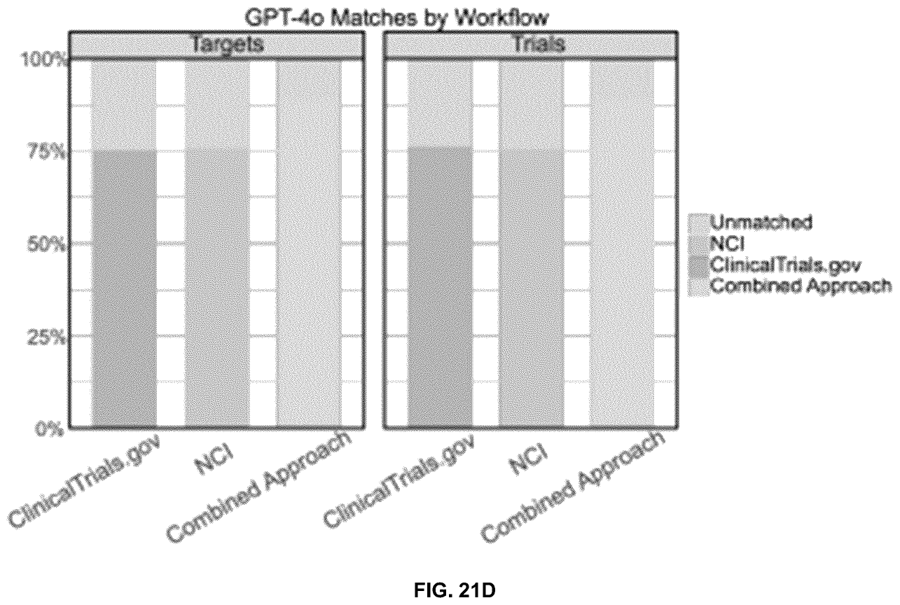

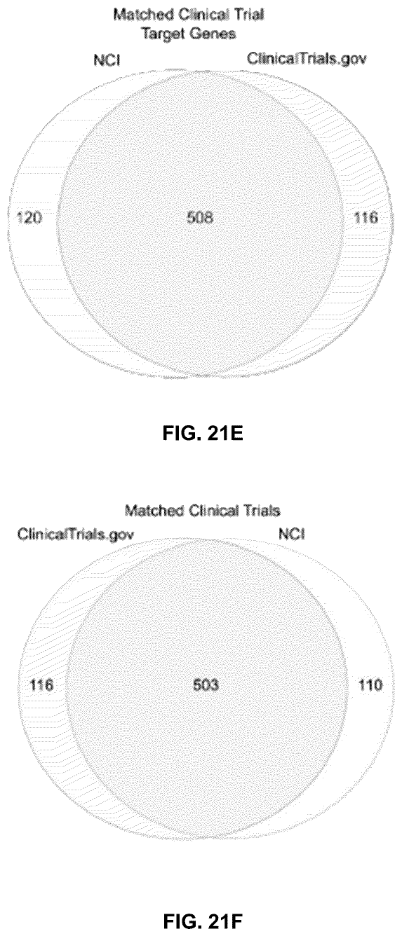

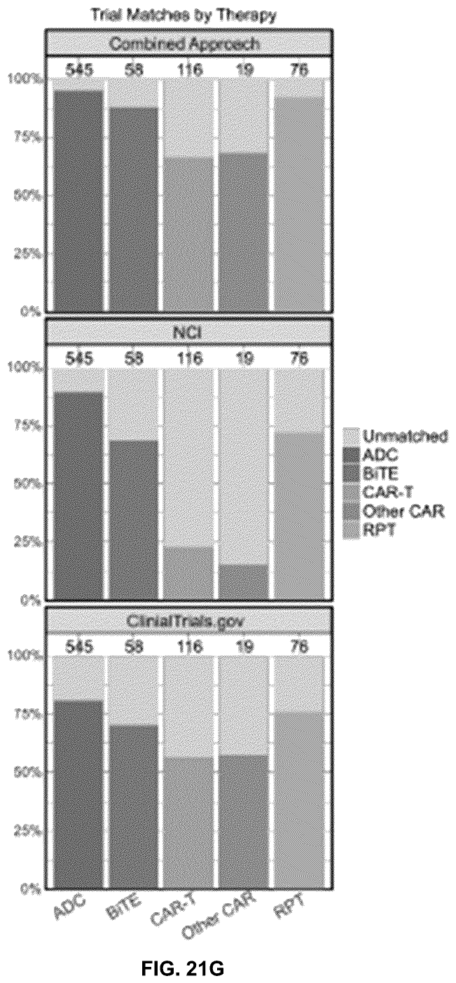

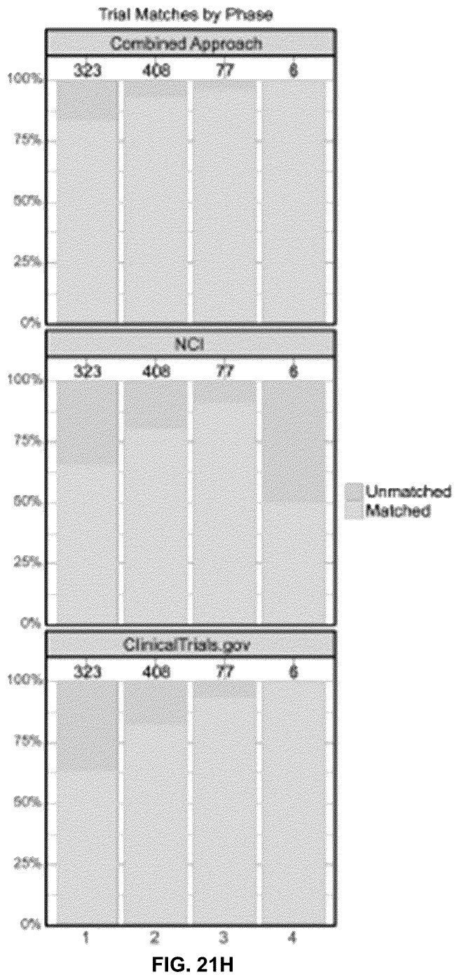

At the time of our study, ClinicalTrials.gov listed over 100,000 trials and the associated intervention, and the NCI Drug Dictionary contained approximately 8,700 drugs and compounds. However, neither of these consistently identify the class or specific target in an indexable format, if at all. There is no up-to-date database that identifies CST clinical trials and their targets. Leveraging the emergent ability of LLMs to read and interpret these text databases can greatly improve the efficiency of identifying CST trials/targets and provide an automated way of creating and maintaining a CST clinical trial and target database^{38,39} (FIG. 20). To achieve this, we first utilized LLMs to identify which trial drugs were CSTs. Then, we utilized a two-pronged approach, prompting each LLM to identify each drug target based on either the drug name in ClinicalTrials.gov or the drug description in the NCI Drug Database (FIG. 21A). To establish a “ground truth” for evaluating LLM performance, we manually curated a list of CST clinical trials and their gene targets up to October 2023²¹. This entire pipeline is automated and can be reproduced on-demand, allowing an up-to-date CST clinical trial and, importantly, target list. The performance benchmarks of our approach are shown below.

### LLM Performance—Identifying CST Trials

The first step in matching patients with trials is to identify which trials utilize CSTs. In total, we investigated six different general “open” models (Llama 3.1:70b, Llama 3.3, Gemma 3:27b, Phi-4, Granite 3.2:8b, and Qwen2.5:72b), as well as an “open” medical LLM (MedLlama3-v20) and a “closed” model (GPT-4o). We selected a range of open models since they can be downloaded for free and run locally, along with the “closed” GPT-4o model, which is widely used with excellent performance across domains. We evaluated these LLMs in a human-curated set of 814 CST trials²¹. We also randomly selected an equal number of trials outside of this list to serve as non-CST controls. GPT-4o performed the best, with the combined approach achieving an overall accuracy of 96.4%. It accurately identified 99.3% of the human-curated CST trials, and 93.5% of the non-CST trials as such (FIG. 21B). Importantly, we assumed that all CST trials were in the manually curated list and that the 814 randomly selected trials were not CST trials, as it was impossible to manually review every trial in ClinicalTrials.gov. However, human curation is imperfect, and so we also manually examined the trials GPT-4o “incorrectly” marked as not a CST trial and found 10 that were actually CST trials. Thus, the adjusted accuracy of GPT-4o rises to 97%.

### LLM Performance—Identifying CST Trial Targets

Since LLMs can accurately identify CSTs from non-CST trials, we next examined if LLMs could also identify the target of the CST trials. To evaluate performance, we looked at both the accuracy in identifying the correct target(s) from the therapies used in each clinical trial (the target-level accuracy, as some trials have more than one CST), as well as if a trial was correctly selected when starting from each CST (the trial-level accuracy). Using the combined approach, the GPT-4o model again had the best performance of those tested, with approximately 89.5% accuracy at both the trial and target-level (FIG. 21C). We also found that Llama 3.1:70b and Llama 3.3 have similar performance to GPT-4o, and both Llama models have very similar performance when compared to each other. These findings are consistent with other work that demonstrates GPT models generally outperform open LLMs in both zero-shot and few-shot clinical trial-related tasks^{7,35,40}.

We found that the combination of the drug name (from ClinicalTrials.gov) and description (from the NCI drug database) yielded better performance than each individually (FIG. 21D). While there were a large number of common matches, LLMs utilizing each data source also find a unique subset of trials (FIGS. 21E-21F). Interestingly, while GPT-4o successfully matches a similar number of therapies using either dataset, other models perform comparatively worse with drug name alone (ClinicalTrials.gov) and derive a greater percentage of overall matches from the description (NCI; FIG. 21D). This suggests that part of GPT-4o's superior performance lies in its general knowledgebase, whereas other models rely more heavily on direct text interpretation.

The class of the CST can also affect target match accuracy. We found that CAR-related therapies generally were the most difficult to correctly identify (FIG. 21G). This is likely indicative of more complicated naming schemes for CAR-related therapies when compared to straightforward and standardized drug names for other CSTs. We also investigated the effect of clinical trial phase, and found that accuracy was lowest for Phase I trials and progressively increased with later phase trials (FIG. 21H). This is most likely due to the more complete information available both in these two databases but also background information on which the LLMs were trained for therapies in late-stage trials.

### Factors Contributing to Targets/Trials Match Errors

We next performed an analysis on the underlying causes of situations where the LLM approach was not able to match a target and clinical trial, focused on the GPT-4o model since it had the best performance. We investigated the 87 target-trial pairs that were not correctly matched by the GPT-4o model using either the drug name alone from ClinicalTrials.gov or the description in the NCI database, and grouped errors into seven categories. 43 errors were caused by vagueness in the therapy description in the clinical trial data (e.g. “Car T cells” without specifying more; deemed “Vague Therapy Name”). 10 errors were caused by similar vagueness in the NCI drug database description (deemed “Vague Therapy Description”). These errors are due to a lack of information and likely would require human updating of the databases to fix. However, other errors were due to the inherent limitations of LLMs, which may be overcome as the models continue to advance. 19 errors were due to the fact that the therapy in the clinical trial did not match any drug in the NCI drug database (deemed “No NCI Match”). 27 errors were caused by string matching challenges for a drug. 14 errors were caused by errors interpreting the correct target in a complex therapy name (deemed “On-Topic”). 38 times, the LLM was not able to identify the target (deemed “No Target Identified”) and 23 errors were hallucinations. Hallucinations are when an LLM generates incorrect but plausible-appearing answers, and are a well-known problem both inside and outside of the healthcare space⁴¹⁻⁴⁴. We did not observe any particular association between the error category and the therapy type. These results emphasize the need for careful data entry at the time of drug or trial registration, as the output of an LLM depends not only on the model itself, but the quality and structure of the information in the prompt⁴⁵⁻⁴⁷.

### Geographic Distribution of LLM Performance

Trial availability is known to be a barrier to patient enrollment, both in terms of physician knowledge of available trials and patient proximity to open trials^{3,5}. Unsurprisingly, of the top five states with the largest number of CST trials in our human-curated list (California, Texas, New York, Florida, Washington), four of these states are in the top five most populous states in the US⁴⁸. The highest rate of target-trial match errors by GPT-4o was 10.53% in South Dakota, though this represented an absolute number of only 2 incorrect trials. Overall, our workflow was able to match a target to approximately 94% of the 483 trials in the United States. Next, we expanded our investigation of model accuracy globally. Notably, not every country will register trials on ClinicalTrials.gov. As expected, the United States had the most trials, followed by China, Spain, France, and Italy. China had the highest rate of trial match errors at 16.17%, with New Zealand as the second highest with a rate of 11.11%. Other than these exceptions, trials in countries outside of the United States had a match rate exceeding 95%.

### LLM-Based Cell Surface Annotation

Accurate annotation of the cell surface localization of potential targets is increasingly important, both for the identification of clinical trial targets but also for drug development efforts. However, experimental evidence does not exist for every gene in every cancer and normal tissue type. Therefore, we sought to utilize LLMs to make predictions on cell surface localization based solely on their existing knowledge. Most LLMs were able to accurately identify almost all CSTs in our human-curated clinical trial list as such. Gemma 3:27b and MedLlama3 had the most accurate results, successfully labeling all the clinical cell surface targets as cell surface-localized. All models performed quite well in this task, with the exception of Granite 3.2:8b, potentially related to its small parameter size.

To investigate this question genome-wide, we chose to focus on Gemma 3:27b as well as the top 3 best models from the target-trial matching task (GPT-4o, Llama 3.3, and Qwen2.5:72b) and the medically-tuned model (MedLlama3). Each model was prompted to label every protein coding gene as cell surface or not. We found that calls across replicates were highly reproducible. We then compared the performance against a benchmark set of experimentally validated CSTs as well as computationally predicted CSTs in 4+ studies. Gemma 3:27b, Llama 3.3, and Qwen2.5:72b tended to more liberally assign cell surface localization, with increased false positive rates relative to our benchmark. This was extremely pronounced in MedLlama3, where nearly every gene was labeled as cell surface. Interestingly, GPT-4o showed a more conservative bias, having a higher rate of false negatives than false positives. Overall, the performance in this task varies significantly based on the model used and demonstrates LLMs have more difficulty in identifying sub-cellular localization, reflecting the real-world difficulty of this task from both an experimental and computational standpoint.

### Discussion

Improved matching of patients to clinical trials is critical to the success of new cancer treatments⁶. The targeted nature and pan-cancer coverage of trials make this particularly challenging for CSTs, as a treating physician would need to know all trials for all targets across all cancers—an impossible task. Trial awareness and availability are thus major barriers to enrollment^{3,5}, and no centralized and up-to-date database exists for CST trials. In this study, we benchmarked LLMs against a human-curated list of CST trials. We show that LLMs can accurately identify CST trials. We then develop a novel LLM-driven approach for identifying the targets of these trials. This approach demonstrates high accuracy, especially for non-Asia-Pacific and later-phase trials. This allows for a fully automated workflow that generates an up-to-date database of CST clinical trials. When this approach is paired with high-throughput methods exist for identifying and confirming potential targets expressed in a patient²¹⁻³³, it can dramatically enable expanded clinical trial options for patients.

We provide this updated list of open phase I-III trials through May 25, 2025 and their target. As an example, if a patient with prostate cancer was identified as expressing Trop2 (aka TACSTD2), then examining this list, we would find 115 potential TACSTD2 trials across a variety of cancers, which can be further filtered down to 20 trials targeting TACSTD2 in prostate cancer or solid tumors.

While the performance of our LLM-approach is excellent compared to a human-curated “ground truth”, it will likely never be perfect. Interestingly, a significant percentage of the “false negatives” with regards to identifying CST trials were later corrected to “true negatives” after additional human review. When the LLM inadvertently assigns a clinical trial incorrectly to a target (or hallucinates), this is often easily identified by the treating physician or clinical trial staff. An error of omission is more problematic, as it is unlikely the physician or patient would be able to identify that this had occurred. However, consider a hypothetical case where five additional trials were correctly identified for a particular patient beyond what the treating physician was aware of, and one potential additional trial was missed by the LLM. This is still five more trials than would have been available previously. Thus, the potential for benefit is high, with a low impact from errors. The rate of mistakes is already low, and will likely continue to improve as the models themselves do.

Matching patients with expression of CSTs with trials targeting those specific targets has potential benefits both for patients and the trials themselves. This is reflected in the eligibility criteria of many CST trials, though even in trials without restricted eligibility, most patients and physicians would still choose a trial for a highly expressed target. Increasing the rate of enrollment to CST trials for patients with expression of the targets can also benefit the trials themselves, as the odds of success may be higher, and a negative trial is more easily interpreted as a failure of the therapy as opposed to simply sub-optimal patient selection. Ultimately, this approach can vastly streamline and grow our ability to identify appropriate clinical trials for patients while simultaneously improving clinical trial enrollment and providing therapies to the patients most likely to derive benefit.

### Methods

### Model Selection

We evaluated a number of “open” models that can all be downloaded and run locally. These include Llama 3.1:70b and Llama 3.3 from Meta⁴⁹, Gemma 3:27b from Google⁵⁰, Phi-4 from Microsoft⁵¹, Granite 3.2:8b from IBM⁵², and Qwen2.5:72b from Alibaba Cloud⁵³. Given its popularity and robust performance in many tasks, we have also included GPT-4o from OpenAI in our evaluations, despite it being a “closed” model with associated costs⁵⁴⁵⁵. We additionally included a model tuned specifically for medical use, MedLlama3v-20⁵⁶⁻⁵⁷.

### Clinical CST Dataset

In our evaluations of target/trial gene target accuracy, we used our previous work on cell surface genes as a “ground truth” list of clinical trials²¹. This list represents over 800 trials that have been manually curated and annotated for the target gene of interest and the type of therapy being investigated (ADCs, RPTs, etc.) by the trial. For matching gene targets to clinical trials, we leveraged both the NCI Drug Dictionary and intervention labels as reported by the clinical trial directly. NCI drug dictionary definitions were collected using the cancer.gov Application Programming Interface (API). Intervention labels were collected via the ClinicalTrials.gov API. The Therapeutic Target Database (TTD)⁵⁸, used to supplement LLM gene target calls, was downloaded from their web interface.

### Prompting and Workflow

For both target calling and cell surface assignment, prompting was performed using APIs. For the Llama 3.1:70b and Llama 3.3, Gemma 3:27b, Phi-4, Granite 3.2:8b, Qwen2.5:72b, and MedLlama3v-20 models, we utilized ollama, a local LLM runtime and model management platform, to conduct our queries. We made use of the ollamar R package to conduct our queries within an R environment⁵⁹. For GPT-4o queries, we utilized the API provided by OpenAI. For all queries across all models, the temperature parameter was set to 0 to generate more deterministic outputs and maximize reproducibility. Prompting was conducted in a zero-shot setting, with response formatting achieved through prompting and downstream parsing. The clinical trial intervention target results and the NCI drug definition target results are then concatenated to create the “combined approach” list.

### Prompting and Workflow—Therapy Type Assignment

For each NCI drug, each model was prompted to identify the type of therapy using either the name of the drug or the drug definition. For each clinical trial, each model was prompted to identify the type of therapy using either the trial title, the trial summary, or the trial interventions. Results from the NCI drug dictionary and clinical trial list were then concatenated.

### Prompting and Workflow—Gene Target Assignment

For each NCI drug definition, each model was prompted to identify the HUGO/HGNC gene name of the drug's target based on the free text description (i.e. the ‘definition’) of the drug as provided by the NC. For each “intervention” provided in our ground truth list of clinical trials, the models were prompted to identify the HUGO/HGNC gene target of the intervention based on name alone. Once both sets of responses were collected, we “refined” the responses. The “refining” step prompts the model to extract gene names from larger strings of text (i.e. when a model erroneously provides a more detailed response than prompted) and to correct common names to proper HUGO/HGNC gene names. Next, we merged manually curated, known drug-target pairs, obtained from the TTD, with our LLM results in order to augment any drugs or interventions for which the LLM was unable to determine a target gene. The next step in our workflow is a “matching” step, in which NCI drug definition results are then merged back with clinical trial information by string matching NCI drug names with clinical trial intervention labels. Results from the NCI data and clinical trial interventions were then concatenated.

### Prompts for Therapy Type Assignment

Below is the prompt template for determining the type of therapy from clinical trial names and summaries as provided by ClinicalTrials.gov. {SUMMARY} represents the trial title or summary.
“You are a clinical trial researcher with expertise in oncology and cellular therapies. Given the following clinical trial summary, identify all applicable types of therapy being investigated. Choose only from the following options: Radiopharmaceutical or Radioligand (return as RPT); Antibody Drug Conjugate or ADC (return as ADC); BITE or bispecific T cell engager (return as BITE); CAR-T; CAR-NK; CAR-DC; CAR-M. Return CAR if it is a CAR-based therapy but the specific cell type is not clear. In cases where there is more than one therapy, return the answer separated by the pipe character (|). If the therapy is not one of the above, return NA. Do not include any explanation or commentary. The clinical trial summary is as follows: {SUMMARY}”

Below is the prompt template for determining the type of therapy from NCI drug and clinical trial intervention names. {THERAPY} represents the drug or intervention name.
“You are a clinical trial researcher with expertise in oncology and cellular therapies. Given therapy or therapies used in the trial, identify all applicable types of therapy being investigated. Choose only from the following options: Radiopharmaceutical or Radioligand (return as RPT); Antibody Drug Conjugate or ADC (return as ADC); BITE or bispecific T cell engager (return as BITE); CAR-T; CAR-NK; CAR-DC; CAR-M. Return CAR if it is a CAR-based therapy but the specific cell type is not clear. In cases where there is more than one therapy, return the answer separated by the pipe character (|). If the therapy is not one of the above, return NA. Do not include any explanation or commentary. The therapy or therapies to label are as follows: {THERAPY}”

Below is the prompt template for determining the type of therapy from NCI drug definitions. {NCI_DEFINITION} represents the text chunk extracted from the NCI drug dictionary.
“You are a clinical trial researcher with expertise in oncology and cellular therapies. Given the following drug description/drug definition, identify the type of therapy. Choose only from the following options: Radiopharmaceutical or Radioligand (return as RPT); Antibody Drug Conjugate or ADC (return as ADC); BITE or bispecific T cell engager (return as BITE); CAR-T; CAR-NK; CAR-DC; CAR-M. Return CAR if it is a CAR-based therapy but the specific cell type is not clear. In cases where there is more than one therapy, return the answer separated by the pipe character (|). If the therapy is not one of the above, return NA. Do not include any explanation or commentary. The drug description/definition is as follows: {NCI_DEFINITION}”

### Cell Surface Localization

For investigations into cell surface calling, gene lists were compiled from a published datasets⁶⁰⁻⁶⁶, GO Term Cell Surface (GO0009986)⁶⁷⁻⁶⁸, and the Human Protein Atlas⁶⁹. We created a “ground truth” cell surface gene dataset by selecting all genes that met at least one of the following criteria: (1) identified as a cell surface gene in an experimental dataset, (2) identified as a cell surface gene in at least four of the seven computational datasets. For cell surface assignment calls, each model was prompted to assign a gene as existing on the cell surface or not, returning the answer as a binary 0/1 response. This was repeated in triplicate for each model.

### Analysis and Visualization

Analyses were performed using R 4.0.4 and R 4.4.3⁷⁰ and the tidyverse⁷¹ packages, as well as Python 3.12.9⁷². Plots were generated via ggplot2⁷³ and patchwork⁷⁴ R packages. The US and world map plots were generated with the rnaturalearth⁷⁵, usmap⁷⁶, and sf⁷⁷ packages. Figures were generated with the help of Biorender.

### REFERENCES

1. Haidich, A.-B. & Ioannidis, J. P. A. Patterns of patient enrollment in randomized controlled trials. J. Clin. Epidemiol. 54, 877-883 (2001).
2. Comis, R. L., Miller, J. D., Aldigd, C. R., Krebs, L. & Stoval, E. Public Attitudes Toward Participation in Cancer Clinical Trials. J. Clin. Oncol. 21, 830-835 (2003).
3. Unger, J. M., Vaidya, R., Hershman, D. L., Minasian, L. M. & Fleury, M. E. Systematic Review and Meta-Analysis of the Magnitude of Structural, Clinical, and Physician and Patient Barriers to Cancer Clinical Trial Participation. JNCI J. Natl. Cancer Inst. 111, 245-255 (2019).
4. Unger, J. M., Cook, E., Tai, E. & Bleyer, A. The Role of Clinical Trial Participation in Cancer Research: Barriers, Evidence, and Strategies. Am. Soc. Clin. Oncol. Educ. Book 185-198 (2016) doi:10.1200/EDBK_156686.
5. Kadam, R. A., Borde, S. U., Madas, S. A., Salvi, S. S. & Limaye, S. S. Challenges in recruitment and retention of clinical trial subjects. Perspect. Clin. Res. 7, 137-143 (2016).
6. Penberthy, L. T., Dahman, B. A., Petkov, V. I. & DeShazo, J. P. Effort Required in Eligibility Screening for Clinical Trials. J. Oncol. Pract. 8, 365-370 (2012).
7. Nievas, M., Basu, A., Wang, Y. & Singh, H. Distilling large language models for matching patients to clinical trials. J. Am. Med. Inform. Assoc. 31, 1953-1963 (2024).
8. Bennette, C. S. et al. Predicting Low Accrual in the National Cancer Institute's Cooperative Group Clinical Trials. JNCI J. Natl. Cancer Inst. 108, djv324 (2015).
9. Duan, X.-P. et al. New clinical trial design in precision medicine: discovery, development and direction. Signal Transduct. Target. Ther. 9, 1-29 (2024).
10. Makawita, S. & Meric-Bernstam, F. Antibody-Drug Conjugates: Patient and Treatment Selection. Am. Soc. Clin. Oncol. Educ. Book 105-114 (2020) doi:10.1200/EDBK_280775.
11. Williams, M., Spreafico, A., Vashisht, K. & Hinrichs, M. J. Patient Selection Strategies to Maximize Therapeutic Index of Antibody-Drug Conjugates: Prior Approaches and Future Directions. Mol. Cancer Ther. 19, 1770-1783 (2020).
12. Kegyes, D. et al. Patient selection for CAR T or BiTE therapy in multiple myeloma: Which treatment for each patient? J. Hematol. Oncol. J Hematol Oncol 15, 78 (2022).
13. Zhu, W. M. & Middleton, M. R. Combination therapies for the optimisation of Bispecific T-cell Engagers in cancer treatment. Immunother. Adv. 3, ltad013 (2023).
14. Sgouros, G., Bodei, L., McDevitt, M. R. & Nedrow, J. R. Radiopharmaceutical therapy in cancer: clinical advances and challenges. Nat. Rev. Drug Discov. 19, 589-608 (2020).
15. Theret, L. et al. Identification of Novel Cell Surface Therapeutic Targets for KMT2A-Rearranged Acute Myeloid Leukemia. Blood 140, 6316-6317 (2022).
16. Schettini, F. et al. Identification of cell surface targets for CAR-T cell therapies and antibody-drug conjugates in breast cancer. ESMO Open 6, 100102 (2021).
17. Schrofelbauer, B. et al. Discovery of antibodies and cognate surface targets for ovarian cancer by surface profiling. Proc. Natl. Acad. Sci. 120, e2206751120 (2023).
18. Shraim, R. et al. ImmunoTar—integrative prioritization of cell surface targets for cancer immunotherapy. Bioinformatics 41, btaf060 (2025).
19. Wang, Y. et al. Comprehensive Surfaceome Profiling to Identify and Validate Novel Cell-Surface Targets in Osteosarcoma. Mol. Cancer Ther. 21, 903-913 (2022).
20. Modi, S. et al. Trastuzumab Deruxtecan in Previously Treated HER2-Low Advanced Breast Cancer. N. Engl. J. Med. 387, 9-20 (2022).
21. Sharifi, M. N. et al. Clinical cell-surface targets in metastatic and primary solid cancers. JCI Insight 9, (2024).
22. Jilani, S. et al. CAR-T cell therapy targeting surface expression of TYRP1 to treat cutaneous and rare melanoma subtypes. Nat. Commun. 15, 1244 (2024).
23. Verkleij, C. P. M. et al. Preclinical activity and determinants of response of the GPRC5DxCD3 bispecific antibody talquetamab in multiple myeloma. Blood Adv. 5, 2196-2215 (2021).
24. Orentas, R. J. et al. Paired Expression Analysis of Tumor Cell Surface Antigens. Front. Oncol. 7, (2017).
25. Ibanez, J. et al. GRP78-CAR T cell effector function against solid and brain tumors is controlled by GRP78 expression on T cells. Cell Rep. Med. 4, 101297 (2023).
26. Goodyear, O. et al. Induction of a CD8+ T-cell response to the MAGE cancer testis antigen by combined treatment with azacitidine and sodium valproate in patients with acute myeloid leukemia and myelodysplasia. Blood 116, 1908-1918 (2010).
27. Criscitiello, C., Morganti, S. & Curigliano, G. Antibody-drug conjugates in solid tumors: a look into novel targets. J. Hematol. Oncol. J Hematol Oncol 14, 20 (2021).
28. Althammer, S. et al. Automated image analysis of NSCLC biopsies to predict response to anti-PD-Li therapy. J. Immunother. Cancer 7, 121 (2019).
29. Wang, X. et al. Computer extracted features of cancer nuclei from H&E stained tissues of tumor predicts response to nivolumab in non-small cell lung cancer. J. Clin. Oncol. 36, 12061-12061 (2018).
30. Beck, A. H. et al. Systematic Analysis of Breast Cancer Morphology Uncovers Stromal Features Associated with Survival. Sci. Transl. Med. 3, 108ra113-108ra113 (2011).
31. Serag, A. et al. Translational AI and Deep Learning in Diagnostic Pathology. Front. Med. 6, (2019).
32. Baxi, V., Edwards, R., Montalto, M. & Saha, S. Digital pathology and artificial intelligence in translational medicine and clinical practice. Mod. Pathol. 35, 23-32 (2022).
33. Barsoum, I., Tawedrous, E., Faragalla, H. & Yousef, G. M. Histo-genomics: digital pathology at the forefront of precision medicine. Diagnosis 6, 203-212 (2019).
34. Layne, E. et al. Large language models for automating clinical trial matching. Curr. Opin. Urol. 35, 250 (2025).
35. Gupta, S. K. et al. PRISM: Patient Records Interpretation for Semantic Clinical Trial Matching using Large Language Models. Preprint (2024).
36. Chowdhury, S. et al. Matching Patients to Clinical Trials using LLaMA 2 Embeddings and Siamese Neural Network. 2024.06.28.24309677 Preprint (2024).
37. Jin, Q. et al. Matching patients to clinical trials with large language models. Nat. Commun. 15, 9074 (2024).
38. Naveed, H. et al. A Comprehensive Overview of Large Language Models. Preprint (2024).
39. Raiaan, M. A. K. et al. A Review on Large Language Models: Architectures, Applications, Taxonomies, Open Issues and Challenges. IEEE Access 12, 26839-26874 (2024).
40. Wornow, M. et al. Zero-Shot Clinical Trial Patient Matching with LLMs. Preprint (2024).
41. Farquhar, S., Kossen, J., Kuhn, L. & Gal, Y. Detecting hallucinations in large language models using semantic entropy. Nature 630, 625-630 (2024).
42. Perkovid, G., Drobnjak, A. & Boticki, I. Hallucinations in LLMs: Understanding and Addressing Challenges. in 2024 47th MIPRO ICT and Electronics Convention (MIPRO) 2084-2088 (2024). doi:10.1109/MIPRO60963.2024.10569238.
43. Shah, S. V. Accuracy, Consistency, and Hallucination of Large Language Models When Analyzing Unstructured Clinical Notes in Electronic Medical Records. JAMA Netw. Open 7, e2425953 (2024).
44. Asgari, E. et al. A Framework to Assess Clinical Safety and Hallucination Rates of LLMs for Medical Text Summarisation. 2024.09.12.24313556 Preprint (2025).
45. Chen, B., Zhang, Z., Langrend, N. & Zhu, S. Unleashing the potential of prompt engineering in Large Language Models: a comprehensive review. Preprint (2024).
46. Sahoo, P. et al. A Systematic Survey of Prompt Engineering in Large Language Models: Techniques and Applications. Preprint at (2024).
47. He, J. et al. Does Prompt Formatting Have Any Impact on LLM Performance? Preprint (2024).
48. U.S. Census Bureau. U.S. Census Bureau QuickFacts: United States.
49. Grattafiori, A. et al. The Llama 3 Herd of Models. Preprint.
50. Gemma Team et al. Gemma 3 Technical Report. Preprint (2025).
51. Abdin, M. et al. Phi-4 Technical Report. Preprint (2024).
52. IBM Research. Granite Foundation Models.
53. Qwen et al. Qwen2.5 Technical Report. arXiv.org.
54. OpenAI et al. GPT-4 Technical Report. Preprint (2024).
55. OpenAI et al. GPT-4o System Card. arXiv.org (2024).
56. JL42/medllama3-v20-GGUF ⋅ Hugging Face.
57. The Open Medical-LLM Leaderboard: Benchmarking Large Language Models in Healthcare. (2025).
58. Zhou, Y. et al. TTD: Therapeutic Target Database describing target druggability information. Nucleic Acids Res. 52, D1465-D1477 (2024).
59. Lin, H. & Safi, T. ollamar: An R package for running large language models. J. Open Source Softw. 10, 7211 (2025).
60. Bausch-Fluck, D. et al. A Mass Spectrometric-Derived Cell Surface Protein Atlas. PLOS ONE 10, e0121314 (2015).
61. Bausch-Fluck, D. et al. The in silico human surfaceome. Proc. Natl. Acad. Sci. 115, E10988-E10997 (2018).
62. da Cunha, J. P. C. et al. Bioinformatics construction of the human cell surfaceome. Proc. Natl. Acad. Sci. 106, 16752-16757 (2009).
63. Donnard, E. et al. Mutational analysis of genes coding for cell surface proteins in colorectal cancer cell lines reveal novel altered pathways, druggable mutations and mutated epitopes for targeted therapy. Oncotarget 5, 9199-9213 (2014).
64. Lee, J. K., Choi, I. S., Oh, T. I. & Lee, E. Cell-Surface Engineering for Advanced Cell Therapy. Chem.-Eur. J. 24, 15725-15743 (2018).
65. Governa, V. et al. Landscape of surfaceome and endocytome in human glioma is divergent and depends on cellular spatial organization. Proc. Natl. Acad. Sci. U.S.A. 119, e2114456119 (2022).
66. Hu, Z. et al. The Cancer Surfaceome Atlas integrates genomic, functional and drug response data to identify actionable targets. Nat. Cancer 2, 1406-1422 (2021).
67. The Gene Ontology Consortium et al. The Gene Ontology knowledgebase in 2023. Genetics 224, iyad031 (2023).
68. Ashburner, M. et al. Gene Ontology: tool for the unification of biology. Nat. Genet. 25, 25-29 (2000).
69. Thul, P. J. et al. A subcellular map of the human proteome. Science 356, eaa13321 (2017).
70. R Core Team. R: The R Project for Statistical Computing. (2021).
71. Wickham, H. et al. Welcome to the Tidyverse. J. Open Source Softw. 4, 1686 (2019).
72. Python Software Foundation. Python programming language. Python.org.
73. Wickham, H. Ggplot2: Elegant Graphics for Data Analysis. (Springer, New York, NY, 2009). doi:10.1007/978-0-387-98141-3.
74. Pedersen, T. L. patchwork: The Composer of Plots. (2024).
75. Massicotte, P., South, A. & Hufkens, K. rnaturalearth: World Map Data from Natural Earth. (2023).
76. Lorenzo, P. D. usmap: US Maps Including Alaska and Hawaii. (2024).
77. Pebesma, E. & Bivand, R. Spatial Data Science: With Applications in R. (Chapman and Hall/CRC, New York, 2023). doi:10.1201/9780429459016.

### Exemplary Embodiments

Exemplary Embodiment 1. A method of generating a prediction model of cell-surface protein expression in cancer, the method comprising: determining a gene expression profile for each training cell sample in a set of training cell samples, wherein each gene expression profile comprises a set of gene-expression values for a set of gene-expression-profile genes, wherein the set of training cell samples comprises a set of training cancer cell samples; determining a set of training genes, wherein the set of training genes comprises a common set of gene-expression-profile genes represented in each of the gene expression profiles, wherein the set of training genes comprises a set of cell-surface protein genes; ranking the gene-expression values for all of the training genes in each expression profile to thereby obtain a training ranking for each gene expression profile, wherein each training ranking comprises a rank for the gene-expression value for each training gene within the gene expression profile; identifying, for each training gene, the training cell samples having a same rank.

Exemplary Embodiment 2. The method of Exemplary Embodiment 1, wherein the ranking comprises ordinally ranking the gene-expression values for all of the training genes in each gene expression profile to thereby obtain an ordinal training ranking for each gene expression profile, wherein each ordinal training ranking comprises an ordinal rank for the gene-expression value for each training gene within the gene expression profile.

Exemplary Embodiment 3. The method of Exemplary Embodiment 2, wherein the ranking further comprises: determining a number of genes in the set of training genes; and transforming each ordinal ranking into a quantile ranking by dividing the ordinal rank for the gene-expression value for each gene within the gene expression profile by the number of genes in the set of training genes.

Exemplary Embodiment 4. The method of any prior Exemplary Embodiment, wherein the gene expression profiles are determined through RNA-Seq, gene expression microarray, or a combination thereof.

Exemplary Embodiment 5. The method of any prior Exemplary Embodiment, wherein the set of cell-surface protein genes comprises of at least 1, at least 5, at least 10, at least 15, at least 20, at least 25, at least 30, at least 35, at least 40, at least 45, at least 50, at least 55, at least 60, at least 65, at least 70, or each of FOLH1, MUC1, MAGEA3, NCAM1, CD276, EPCAM, GPNMB, ERBB2, CEACAM5, CD70, CA6, SLC44A4, TNC, MSLN, TNFRSF8, EGFR, GUCY2C, FOLR1, TACSTD2, TACSTD2, ENPP3, MET, DLL3, SLC39A6, F3, EFNA4, NECTIN4, KIT, GPA33, FGFR3, PROM1, LRRC15, ROR1, PSCA, HAVCR1, TFRC, GPC3, CDH6, ERBB3, AXL, ALCAM, PTK7, SLC34A2, SSTR2, LY75, ROR2, MUC16, CD46, ISG20, IGF1R, GRPR, ROBO1, TNFRSF10B, TNFRSF9, STEAP1, CLDN18, ITGB6, ICAM1, TPBG, TYRP1, CD22, CD274, HLA-G, CD83, CLDN6, VTCN1, FAP, FLT1, CXCR4, EPHA2, CA9, EPHA5, PMEL, TM4SF1, ADAM9, CD38, FN1, CEACAM6, and NCR2.

Exemplary Embodiment 6. The method of any prior Exemplary Embodiment, wherein the set of training genes further comprises housekeeping genes.

Exemplary Embodiment 7. The method of any prior Exemplary Embodiment, wherein the set of training genes comprises at least 500 genes, at least 1,000 genes, at least 2,500 genes, at least 5,000 genes, at least 7,500 genes, at least 10,000 genes, at least 12,500 genes, at least 15,000 genes, at least 17,500 genes, or at least 19,000 genes.

Exemplary Embodiment 8. The method of any prior Exemplary Embodiment, wherein the set of training cancer cell samples comprise primary tumor cell samples, metastatic tumor cell samples, or both primary tumor cell samples and metastatic tumor cell samples.

Exemplary Embodiment 9. The method of any prior Exemplary Embodiment, wherein the training cancer cell samples comprise a type of at least 1, at least 5, at least 10, at least 15, at least 20, at least 25, at least 30, at least 35, at least 40, at least 45, or each of adrenocortical carcinoma, anal squamous cell carcinoma, bladder urothelial carcinoma, breast invasive carcinoma (basal subtype), breast invasive carcinoma (HER2 subtype), breast invasive carcinoma (luminal A subtype), breast invasive carcinoma (luminal B subtype), cholangiocarcinoma, colorectal adenocarcinoma, cervical squamous cell carcinoma, cancer of unknown primary, diffuse large B-cell lymphoma, endocervical adenocarcinoma, esophagus squamous cell carcinoma, fibrolamellar carcinoma, glioblastoma multiforme, gastrointestinal stromal tumor, adenoid cystic carcinoma, head and neck squamous cell carcinoma, kidney chromophobe, kidney renal clear cell carcinoma, kidney renal papillary cell carcinoma, acute myeloid leukemia, brain lower grade glioma, liver hepatocellular carcinoma, lung adenocarcinoma, lung squamous cell carcinoma, mesothelioma, miscellaneous, prostate neuroendocrine carcinoma, clear cell ovarian cancer, ovarian serous cystadenocarcinoma, pancreatic adenocarcinoma, pheochromocytoma and paraganglioma, prostate adenocarcinoma, sarcoma, small bowel adenocarcinoma, small cell lung cancer, other small cell or neuroendocrine tumor, skin cutaneous melanoma, testicular germ cell tumor, thyroid carcinoma, thymoma, uterine corpus endometrial carcinoma, uterine carcinosarcoma, esophagus/stomach adenocarcinoma, and uveal melanoma.

Exemplary Embodiment 10. The method of any prior Exemplary Embodiment, wherein the set of training cell samples further comprises training non-cancer cell samples.

Exemplary Embodiment 11. The method of any prior Exemplary Embodiment, wherein the training cell samples include cell lines.

Exemplary Embodiment 12. The method of any prior Exemplary Embodiment, wherein the training cell samples comprise single cell samples.

Exemplary Embodiment 13. The method of Exemplary Embodiment 11, wherein the single cell samples comprise single cancer cells and single non-cancer cells.

Exemplary Embodiment 14. The method of Exemplary Embodiment 1, further comprising: generating a matrix data structure comprising a plurality of rows as the training cell samples and columns as the cancer cell samples; and storing the matrix data structure in computer memory.

Exemplary Embodiment 15. The method of Exemplary Embodiment 14, wherein the matrix data structure is stored in contiguous memory in RAM.

Exemplary Embodiment 16. The method of Exemplary Embodiment 14, further comprising communicating the matrix data structure from the computer memory to a networked database.

Exemplary Embodiment 17. The method of Exemplary Embodiment 1, further comprising, prior to determining the gene expression profile for each training cell sample, pre-processing the set of training cell samples, wherein pre-processing comprises normalizing for gene length and sequencing depth.

Exemplary Embodiment 18. The method of Exemplary Embodiment 17, wherein the normalizing is based on at least one of Transcripts Per Kilobase Million (TPM), Fragments Per Kilobase Million (FPKM), or Reads Per Kilobase Million (RPKM).

Exemplary Embodiment 19. The method of Exemplary Embodiment 1, further comprising, prior to ranking the gene-expression values for all of the training genes in each expression profile, normalizing for gene length and sequencing depth.

Exemplary Embodiment 20. The method of Exemplary Embodiment 19, wherein the set of training cell samples is missing at least one piece of data, and wherein the normalizing accounts for the missing at least one piece of data.

Exemplary Embodiment 21. A method of using the prediction model of any prior claim to determine a predicted cell-surface target on a patient cancer cell sample, the method comprising: ranking gene-expression values of all of the training genes in the patient cancer cell sample to obtain a patient ranking; selecting one or more training genes having a threshold rank in the patient ranking to thereby obtain one or more patient genes; comparing the rank(s) of the one or more patient genes in the patient cancer cell sample with the rank(s) of the one or more patient genes in the training cell samples to determine a number, type, and/or proportion of training cancer cell samples having the same rank(s) for the one or more patient genes; and optionally, empirically testing cell-surface expression of the one or more patient genes in a test cancer cell sample from the patient.

Exemplary Embodiment 22. The method of Exemplary Embodiment 21, wherein the comparing further comprises determining a number, type, and/or proportion of non-cancer cell samples having the same rank(s) for the one or more patient genes.

Exemplary Embodiment 23. The method of Exemplary Embodiment 21, further comprising, prior to ranking the gene-expression values for all of the training genes in each expression profile, normalizing for gene length and sequencing depth.

Exemplary Embodiment 24. The method of Exemplary Embodiment 23, wherein the set of training cell samples is missing at least one piece of data, and wherein the normalizing accounts for the missing at least one piece of data.

Exemplary Embodiment 25. The method of Exemplary Embodiment 21, further comprising: presenting a graphical user interface to a user of the compared rank(s) of the one or more patient genes in the patient cancer cell sample with the rank(s) of the one or more patient genes in the training cell samples as a plurality of top cell surface targets organized by percentile.

Exemplary Embodiment 26. The method of Exemplary Embodiment 25, further comprising generating a pseudo-dynamic graphical user interface that simulates a dynamic user experience by: pre-generating an image of all possible integer percentiles for a given training cancer cell as a plurality of images; and populating the graphical user interface with a given image based on user input of an inputted training gene and an inputted percentile.

Exemplary Embodiment 27. The method of Exemplary Embodiment 26, wherein the graphical user interface is populated with a given image without using an index data structure.

Exemplary Embodiment 28. The method of Exemplary Embodiment 26, wherein each of the plurality of images comprise a filename including training gene and percentile.

Exemplary Embodiment 29. A method of using the prediction model of any one of Exemplary Embodiments 1-20 to identify a cancer type expressing a cell-surface protein, the method comprising: determining the ranks of one of the cell-surface protein genes across the training cancer cell samples of different types; selecting one of the different types of training cancer cell samples having a threshold rank for the one of the cell-surface protein genes; and optionally, empirically testing cell-surface expression of the one of the cell-surface protein genes in a test cancer cell sample.

Exemplary Embodiment 30. A method of using the prediction model of Exemplary Embodiment 11 to identify a model cell line for testing a cell-surface targeted therapy in a particular cancer cell type, the method comprising: identifying types of the training cancer cell samples having a threshold rank of one of the cell-surface protein genes; identifying a first set of cell lines from the training cell samples having a rank of the one of the cell-surface protein genes comparable to the identified types of the training cancer cell samples; identifying a second set of cell lines from the training cell samples that have a threshold level of protein expression of one of the cell-surface protein genes; identifying the cell lines common to the first set of cell lines and the second set of cell lines to thereby obtain one or more model cell lines; and optionally, testing a therapy targeting the cell-surface protein gene on the one or more model cell lines.

Exemplary Embodiment 31. A method of using the prediction model of any one of Exemplary Embodiments 12 and 13 to predict response and/or toxicity in the treatment of a particular type of cancer, the method comprising: identifying a proportion of single cells of a training cancer cell sample from the particular type of cancer having higher than a threshold ranking of one of the cell-surface protein genes; and/or identifying a proportion of single cells of a training non-cancer cell sample having lower than a threshold ranking of the one of the cell-surface protein genes; and optionally, treating the particular type of cancer with a therapeutic targeting the one of the cell-surface protein genes if the proportion of single cells of the training cancer cell sample from the particular type of cancer is higher than a threshold and/or if the proportion of single cells of the training non-cancer cell sample is lower than a threshold.

Exemplary Embodiment 32. A method of using the prediction model of any one of Exemplary Embodiments 1-20 to identify two or more cell-surface targets on a particular type of cancer, the method comprising: generating a logistic regression model for every pair of the cell-surface protein genes comparing each type of training cancer cell sample and each type of training non-cancer cell sample; identifying pairs in which both cell-surface protein genes in the pair individually contribute to predicting a training cancer cell sample versus a training non-cancer cell sample to obtain predictive pairs; and identifying predictive pairs in which the combination of both cell-surface protein genes in the pair discriminate between a training cancer cell sample versus a training non-cancer cell sample.

Exemplary Embodiment 33. A non-transitory computer-readable medium comprising instructions that, when executed by a processor, cause the processor to implement any one of Exemplary Embodiments 1-32.

Exemplary Embodiment 34. A system comprising: at least one processor and memory operably coupled to the at least one processor, and instructions that, when executed on the at least one processor, cause the at least one processor to implement: a user input engine configured to receive clinical or research-based whole-transcriptome RNA-sequence (RNA-seq) data as inputted data and conduct pre-processing of the inputted data to generate a pre-processed gene sequence matrix of expression levels of genes within each sample, a data normalization engine configured to normalize data within the pre-processed gene sequence matrix and determine an ordinal rank order of all genes within each sample, a target identification engine configured to conduct subset analysis of the ordinal rank ordered gene sequence matrix and determine cell surface targets (CSTs), a data distribution engine configured to generate at least one distribution using the CSTs, and a comparison and interactive display engine configured to generate a graphical user interface including a percentile for each CST in a specific patient compared to an overall distribution of tumors or normal tissues.

What is claimed is:

1. A method of generating a prediction model of cell-surface protein expression in cancer, the method comprising:
determining a gene expression profile for each training cell sample in a set of training cell samples, wherein each gene expression profile comprises a set of gene-expression values for a set of gene-expression-profile genes, wherein the set of training cell samples comprises a set of training cancer cell samples;
determining a set of training genes, wherein the set of training genes comprises a common set of gene-expression-profile genes represented in each of the gene expression profiles, wherein the set of training genes comprises a set of cell-surface protein genes;
ranking the gene-expression values for all of the training genes in each expression profile to thereby obtain a training ranking for each gene expression profile, wherein each training ranking comprises a rank for the gene-expression value for each training gene within the gene expression profile;
identifying, for each training gene, the training cell samples having a same rank.

2. The method of claim 1, further comprising:
generating a matrix data structure comprising a plurality of rows as the training cell samples and columns as the cancer cell samples; and
storing the matrix data structure in computer memory.

3. The method of claim 2, wherein the matrix data structure is stored in contiguous memory in RAM.

4. The method of claim 2, further comprising:
communicating the matrix data structure from the computer memory to a networked database.

5. The method of claim 1, further comprising:
prior to determining the gene expression profile for each training cell sample, pre-processing the set of training cell samples, wherein pre-processing comprises normalizing for gene length and sequencing depth.

6. The method of claim 5, wherein the normalizing is based on at least one of Transcripts Per Kilobase Million (TPM), Fragments Per Kilobase Million (FPKM), or Reads Per Kilobase Million (RPKM).

7. The method of claim 1, further comprising:
prior to ranking the gene-expression values for all of the training genes in each expression profile, normalizing for gene length and sequencing depth.

8. The method of claim 7, wherein the set of training cell samples is missing at least one piece of data, and wherein the normalizing accounts for the missing at least one piece of data.

9. The method of claim 1, wherein the ranking comprises ordinally ranking the gene-expression values for all of the training genes in each gene expression profile to thereby obtain an ordinal training ranking for each gene expression profile, wherein each ordinal training ranking comprises an ordinal rank for the gene-expression value for each training gene within the gene expression profile.

10. The method of claim 9, wherein the ranking further comprises:
determining a number of genes in the set of training genes; and
transforming each ordinal ranking into a quantile ranking by dividing the ordinal rank for the gene-expression value for each gene within the gene expression profile by the number of genes in the set of training genes.

11. The method of claim 1, wherein the set of training cell samples further comprises training non-cancer cell samples.

12. The method of claim 1, wherein the training cell samples comprise single cell samples.

13. The method of claim 12, wherein the single cell samples comprise single cancer cells and single non-cancer cells.

14. A method of using a prediction model to determine a predicted cell-surface target on a patient cancer cell sample, the method comprising:
ranking gene-expression values of all of a plurality of training genes in the patient cancer cell sample to obtain a patient ranking;
selecting one or more training genes having a threshold rank in the patient ranking to thereby obtain one or more patient genes;
comparing the rank(s) of the one or more patient genes in the patient cancer cell sample with the rank(s) of the one or more patient genes in the training cell samples to determine a number, type, and/or proportion of training cancer cell samples having the same rank(s) for the one or more patient genes; and
optionally, empirically testing cell-surface expression of the one or more patient genes in a test cancer cell sample from the patient.

15. The method of claim 14, further comprising:
prior to ranking the gene-expression values for all of the training genes in each expression profile, normalizing for gene length and sequencing depth.

16. The method of claim 15, wherein the set of training cell samples is missing at least one piece of data, and wherein the normalizing accounts for the missing at least one piece of data.

17. The method of claim 14, further comprising:
presenting a graphical user interface to a user of the compared rank(s) of the one or more patient genes in the patient cancer cell sample with the rank(s) of the one or more patient genes in the training cell samples as a plurality of top cell surface targets organized by percentile.

18. The method of claim 17, further comprising:
generating a pseudo-dynamic graphical user interface that simulates a dynamic user experience by:
pre-generating an image of all possible integer percentiles for a given training cancer cell as a plurality of images; and
populating the graphical user interface with a given image based on user input of an inputted training gene and an inputted percentile.

19. The method of claim 18, wherein the graphical user interface is populated with a given image without using an index data structure.

20. The method of claim 18, wherein each of the plurality of images comprise a filename including training gene and percentile.
